(19) United States
(12) Patent Application Publication
(10) Pub. No.: US 2010/0329986 A1
(43) Pub. Date: Dec. 30, 2010

(54) Adult Hepatic Progenitor Cells and Methods of Use Thereof

(76) Inventors: Linda E. Greenbaum, Wynnewood, PA (US); Klaus H. Kaestner, Swarthmore, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/683,084

(22) Filed: Jan. 6, 2010

Related U.S. Application Data
(60) Provisional application No. 61/142,747, filed on Jan. 6, 2009.

(51) Int. Cl. A61K 49/00; C12N 5/071; G01N 33/566; A61K 35/407; A61P 1/16

(52) U.S. Cl. 424/9.2; 435/325; 435/395; 435/7.21; 424/93.7; 424/9.1

(57) ABSTRACT

Compositions and methods effective for identifying, isolating, and utilizing adult hepatic progenitor cells are provided.

This application claims priority under 35 U.S.C. §119(e) to U.S. Provisional Application, 61/142,747, filed Jan. 6, 2009, the entire content of which is incorporated herein by reference.

Pursuant to 35 U.S.C. §202(c) it is acknowledged that the U.S. Government has certain rights in the invention described, which was made in part with funds from NIH/NIDDK grant DK R01-053839 and P-30-DK050306.

### FIELD OF THE INVENTION

This invention relates to the field of molecular biology and treatment of disease. More specifically, the present invention provides compositions for identifying, isolating, and utilizing adult hepatic stem cells in drug discovery and therapeutic methods.

### BACKGROUND OF THE INVENTION

Several publications and patent documents are cited throughout this application in order to more fully describe the state of the art to which this invention pertains. The disclosure of each of these citations is incorporated by reference herein.

In the normal liver, the majority of hepatocytes and biliary epithelial cells (cholangiocytes) are quiescent. In response to liver injury or loss of liver mass, proliferation of mature liver cells represents the first-line defense to restore homeostasis. However, in patients and animal models with chronic liver disease, hepatocytes and cholangiocytes are blocked from proliferating and small bipotential progenitors are activated (1-5). These facultative stem cells, described in rodent models as “oval cells” or “intermediate hepatobiliary cells”, have the appearance of small hepatocytes with scant cytoplasm and are postulated to arise from a niche close to the terminal bile ducts, called the canal of Hering. Oval cells are frequently found in “ductular reactions” present in both experimental models of liver injury and in patients with chronic liver disease (6, 7). Their presence is strongly associated with impaired proliferation of mature hepatocytes, indicating that they represent a reserve hepatic progenitor cell population. Repopulation studies in animal models indicate that these progenitors possess the potential to restore function in patients with both acute and chronic liver disease (8).

A better understanding of the pathways involved in the differentiation of hepatic stem cells and the role these cells play during liver recovery requires molecular tools for their isolation and characterization. Likewise, methods for the isolation and ex vivo expansion of human hepatic stem cells are necessary if this approach is to be used in regenerative medicine.

For severe liver disease and liver failure, organ transplantation is currently the only therapeutic option. However, its application is limited by the severe shortage of organ donors. Because the liver possesses the capacity to regenerate, at least to some extent, partial liver transplant procedures have had some success. In order to be able to treat the large number of eligible patients, other avenues, such as the expansion and differentiation of liver stem cells need to be developed. However, these efforts are hampered by the lack of truly specific markers for hepatic progenitor or stem cells. One advantage of certain embodiments of the invention provides such a marker.

### SUMMARY OF THE INVENTION

In accordance with the present invention, isolated adult hepatic progenitor cells expressing Foxl1 and compositions comprising the same are provided. The cells may also express at least one marker selected from the group consisting of stem cell factor, CK19, CK7, DIWPref-I, ABCG2, c-kit, thy-I, CD34, OV6, NCAM, EpCAM, Trop2, and alpha fetoprotein.

In another embodiment, methods for preparing biocompatible polymeric matrix structures for the replacement or repair of a liver are provided by depositing isolated adult hepatic progenitor cells described herein onto the matrix to facilitate proliferation

In another embodiment of the invention, methods are provided for identifying hepatic progenitor cells present in adult tissues, and optionally, methods for isolating the same.

Another aspect of the invention includes methods of treating hepatic injury in a patient in need thereof, comprising administration of an effective number of adult hepatic progenitor cells to the patient. In one aspect, the stem cells are autologous. In another, the cells are provided by a compatible donor.

Methods are also provided for identifying an agent that modulates Foxl1 activity in a cell by providing a hepatic cell expressing Foxl1 activity. An exemplary method entails exposing said cell to a test agent; determining the Foxl1 activity in the cell; and comparing the Foxl1 activity to that of a control cell not exposed to the test agent, wherein a higher or lower Foxl1 activity than that of control indicates that the agent modulates Foxl1 activity. In another embodiment, the method is performed in vivo, preferably in a mouse model. Such mice may optionally have at least one gene modulated by Foxl1 knocked out. Preferably, the gene to be deleted is selected from the group consisting of hedgehog receptor smoothened, wnt, or notch receptor.

In another aspect of the invention, a method for assessing the contribution of hepatic progenitor cells to liver homeostasis is provided. An exemplary method entails introducing a Foxl1-diphtheria toxin receptor BAC transgene into a transgenic founder line, and introducing diphtheria toxin into said mice, thereby selectively ablating Foxl1 expressing cells. Control mice and treated mice are then subjected to conditions that induce liver damage; and the mice are assessed for liver repair in the presence and absence of Foxl1 expression thereby determining the contribution of Foxl1 expressing cells to the proliferative response following liver injury.

Finally, the invention also includes a method for determining whether Foxl1 expressing hepatic progenitor cells give rise to liver cancer following genotoxic or chemotoxic damage. An exemplary method entails labeling Foxl1 expressing cells in a mouse in vivo (e.g., with YFP) exposing the mice to a carcinogen thereby inducing tumor formation; and determining whether cells in the tumor contain the Foxl1 labeled cells, thereby identifying Foxl1 progenitor cells as liver cancer stem cells.

### BRIEF DESCRIPTION OF THE FIGURES

FIG. 1: Foxl1 Sequence. (A) cDNA sequence of full-length mouse Foxl1 (SEQ ID NO: 1). (B) Protein sequence of full-length mouse Foxl1 (SEQ ID NO: 2). (C) Human full-length Foxl1 cDNA (SEQ ID NO: 3). (D) Human full-length Foxl1 protein sequence (SEQ ID NO: 4).

FIG. 2: Foxl1 expression is induced following cholestatic liver injury

(A) Expression of Foxl1 mRNA was significantly increased in livers 5 and 14 days following bile duct ligation (BDL). 5 day (n=4), 2 week (n=4), WT sham n=2. *P-value <0.02. (B-D) Laser capture microdissection of liver tissue following BDL detects Foxl1 mRNA in portal tracts but not the liver parenchyma. Portal tracts and adjacent parenchyma of Foxl1 WT livers following 3, 5 and 7 day BDL, Foxl1 null 5 day BDL and 7 day sham WT BDL were microdissected and RNA isolated for gene expression analysis. (B) Representative portal tract isolated by laser capture microdissection for gene expression analysis. (C) qRT-PCR for Foxl1 establishes that Foxl1 is expressed in the portal tract but not liver parenchyma of BDL mice. Portal tracts of Foxl1 null mice or sham-operated animals did not express Foxl1. (D) qRT-PCR confirmed expression of the bile duct marker CK19 in portal tracts but not in surrounding parenchyma of livers after BDL. Abbreviations, WT (Wild type), PT (Portal Tract), P (Parenchyma), Sh (Sham).

FIG. 3: Foxl1-Cre does not mark fetal hepatoblasts. β-gal staining of Rosa26R control embryo (A) and Foxl1-Cre; Rosa26R embryos (B-D) at day 12.5 of gestation. A) Control embryo with only some blue staining near the otic vesicle. B) Foxl1-Cre; Rosa26R embryo with β-gal positive cells in the developing spinal cord, but none in the liver (marked by dark arrow). C) Section of a 12.5 dpc Foxl1-Cre; Rosa26R embryo, showing β-gal positive cells in the gastrointestinal mesenchyme (light arrow) but not in the liver (dark arrow). D) Another view of the embryo sectioned in C, showing that there are no cells present in the liver wherein the Foxl1-Cre transgene is active in fetal hepatoblasts.

FIG. 4: Lineage tracing of Foxl1-positive cells following bile duct ligation. Foxl1Cre; Rosa26RlacZ mice underwent BDL and livers were harvested 3, 5, 7 and 14 days later. Livers were analyzed for β-gal activity reflecting Foxl1-Cre activation (blue) and co-stained with the cholangiocyte marker CK19 (brown). β-gal positive cells were seen as early as 3 days in β-gal⁺/CK19⁺ co-labeled bile ductular cells (arrowhead) and β-gal⁺/CK19⁻ cells in periportal regions (arrow). By 5 days (C, D) and 7 days (E, F) increasing numbers of β-gal⁺/CK19″ cells were observed (arrow) in periportal regions and the emergence of β-gal stained cells with the morphologic appearance of hepatocytes (F, G, chevron arrow). Figure (A) 5 day sham, 20× magnification, (B) 3 day BDL 40× magnification, (C, D) 5 day BDL 40× magnification, (E, F) 7 day BDL 20× magnification, (G,H) 14 day BDL, 40× magnification. Sequential stain of 14 day BDL at 20× magnification with β-gal (I) followed by CK19 (J). Representative β-gal⁺/CK19⁺ cells are indicated by arrows.

FIG. 5: Lineage tracing of Foxl1-Cre-positive cells following bile duct ligation. Triple labeling for β-gal, CK19 and HNF4α allowed the identification of Foxl1-Cre-lineage positive cholangiocytes (β-gal/CK19 double positive cells) and hepatocytes (β-gal/HNF-4α double positive cells). Liver cells were identified with the hepatocyte marker HNF4α (grey) and cholangiocyte marker CK19 (brown), and Foxl1-Cre-positive cells and their descendants by β-gal staining (blue). Rare double-labeled (β-gal/CK19 cells appeared in sham operated livers (A) and were frequently observed in ductular reactions beginning at 3 days post-BDL and throughout the time course examined (B-F, arrowhead). Single-labeled β-gal positive cells in the 14 day BDL liver (Panel F) are identified by asterisk. Double labeled β-gal/HNF-4α hepatocytes are shown in day 5 post-BDL livers (arrows in C, D, and E). (A) Sham-operated, (B) 3 day BDL, (C) 5 day BDL, (D) 7 day BDL, (E, F) 14 day BDL, 40× magnification. Quantification of percent β-gal⁺/CK19⁺ cells (G) and percent β-gal⁺ and β-gal⁺HNF-4a⁺/total cells at indicated time points post-BDL (H). *p-value <0.05 relative to control, **p-value <0.001 relative to control. ##p-value <0.05 percent β-gal^{+/}total cells relative to β-gal/HNF-4⁺/total cells 7 day BDL.

FIG. 6: Foxl1 is required for bile duct proliferation following BDL. CK-19 staining demonstrates decreased biliary proliferation in foxl1^{-/-} (A) relative to foxl1^{+/+} livers 14D post-BDL (B). (10× magnification).

FIG. 7: The Foxl1-Cre-positive cell lineage is enriched for proliferating cells. β-gal and Ki-67 double-labeling reveals co-localization of β-gal and Ki-67 (brown labeled nuclei), indicating that Foxl1-Cre positive cells are actively proliferating following BDL injury. Only rare Ki-67 positive hepatocytes are detected in sham-operated livers (A, arrow). In the 3-day BDL liver (B, arrowhead)), Ki-67 and β-gal co-localized in cells within bile ductules. Co-localization of Ki-67 and β-gal in 5 (C) and 7-day BDL livers (E) was detected in ductular reactions (arrowheads). Dual stained β-gal/Ki-67 cells with hepatocyte morphology were seen in 5 day BDL (D, arrows). By 14 days post-BDL, the majority of β-gal cells were Ki-67 negative (F-H). (A) sham, 20×, (B) 3 d BDL, 40× (C, D) 5 d BDL 40×, (E) 7 d BDL (40×) and, (F) 14 d BDL, 40×, (G-H), 14 d BDL, 20× magnification.

FIG. 8: The Foxl1-Cre-positive lineage in DDC-treated mice. (A) Expression of Foxl1 mRNA was significantly increased in livers of mice fed a diet containing 0.01% 3,5-diethoxycarbonyl-1,4-dihydrocollidine (DDC). Triple label staining of liver sections of Foxl1-Cre; Rosa26RlacZ mice: β-gal (blue), CK19 (brown) and HNF-4α (gray) 3 days (B), 7 days (C), 14 days (D), or 21 days (E, F) after treatment with a DDC-containing diet. Cells within ductular reactions were co-labeled with β-gal and CK19 beginning 3 days after initiation of DDC diet (B, black arrowhead) and detected throughout the 21-day timecourse (C-F). Co-localization of β-gal and HNF4α is detected in 14 day and 21 day DDC diet livers (yellow arrowhead, D, F). Brown background staining represents heme-containing breakdown products of DDC. Quantitation of percent β-gal⁺/CK19⁺ cells (G) and percent β-gal⁺ and β-gal⁺HNF-4α⁺/total cells (H). *p-value <0.05 relative to control, **p-value <0.01 relative to control. Sequential stain of 21 day DDC 20× magnification with β-gal (I) followed by CK19 (J). Representative β-gal⁺/CK19⁺ cells are indicated by arrows.

FIG. 9: β-gal and CK19 co-expression in mice receiving a choline deficient, ethionine supplemented diet. Panels A and B show that β-gal and CK19 were co-expressed in a subset of CK19 cells in mice receiving CDE diet.

FIG. 10: Foxl1-Cre does not co-localize with myofibroblastic cell markers in the DDC injury paradigm. β-gal/desmin, β-gal/elastin and β-gal/α-SMA co-staining in control (A, C, E) and 21 day DDC diet treated mice (B, D,F). β-gal staining appears aqua blue, while desmin, elastin and SMA are shown as royal blue pseudocolorization of the original immunofluorescence images. Elastin, α-SMA and desmin staining in control livers were confined to perivascular cells (A, C, E). Elastin positive cells were observed in close proximity to, but not overlapping with the β-gal positive cells in DDC fed mice (B, arrow). There was minimal expression of desmin and α-SMA in the DDC injured livers (D, F). 20× magnification.

FIG. 11: Expression of elastin, desmin and α-SMA in BDL injured livers. Panels A and B represent detection of elastin in sham operated and BDL mice, respectively. Panels C and D represent detection of SMA in sham operated and BDL mice, respectively. Panels E and F represent desmin detection in sham operated and BDL mice respectively.

FIG. 12: Foxl1-Cre expressing cells are encircled by portal fibroblasts. Foxl1-Cre and Elastin staining using the Foxl1-Cre and Rosa26R mouse lines. Aqua blue, beta-galactosidase staining in mice after three weeks of DDC diet. In these cells, the Foxl1-Cre had been active (arrow). Royal blue cells express elastin (arrowhead). Brown represents porphyrin DDC breakdown product.

FIG. 13: PCR analysis of founders for the new Foxl1CreERT2 transgenic line. Primers specific for the CreERT2 transgene were used for PCR of tail DNA. Mice #67 and 68 are positive.

FIG. 14: Labeling of the Foxl1-Cre lineage in RosaYFP mice. Foxl1-Cre; RosaYFP mice were fed a DDC-containing diet for 14 days and the animals analyzed by immunofluorescence staining for YFP expression (red). DAP stains nuclei blue. As expected, multiple cells contained in ductular reactions in the portal triad were labeled by the Foxl1-Cre transgene in this model.

FIG. 15: FACS sort of YFP positive cells from DDC treated mice. Foxl1-Cre; RosaYFP mice were fed a DDC-containing diet for 14 days. Nonparenchymal cells were isolated by perfusion of the liver with a series of proteases and subjected to FACS for YFP and the hematopoietic marker CD45. The yellow shaded curve represents total cells.

FIG. 16: Foxl1 marked cells are capable of self-renewal. Nonparenchymal cells from Foxl1Cre;RosaYFP livers 3 days after DDC-diet treatment were enriched for YFP by FACS sorting and cultured for 23 days. Representative colony is shown.

FIG. 17: Foxl1⁺ cells are enriched for other progenitor cell markers. Q-RT-PCR for progenitor cell markers, normalized to HPRT. NPC: total nonparenchymal fraction. YFP-: YFP-fraction of FACS sorted cell fraction. YFP⁺: Foxl1Cre;YFP⁺ sorted cell fraction.

FIG. 18: Design of the Foxl1-Diphtheria Toxin Receptor BAC transgenic mouse. The Foxl1-BAC was modified by “recombineering” to replace the Foxl1 coding sequence with that of the human HBEGF cDNA, the receptor for Diphtheria Toxin.

FIG. 19: PCR analysis of transgenic founders carrying the Foxl1-hDTR BAC transgene. Primers were specific to the human HBEGF gene, the receptor for diphtheria toxin.

FIG. 20: Tyrosine catabolism. In FAH deficiency, succinylacetone and its toxic metabolites accumulate in hepatocytes. NTBC inhibits 4-hydroxyphenylpyruvate dioxygenase (Step 2), and prevents succinylacetone formation (from Grompe et al., 1995).

FIG. 21: Principle of Diphtheria Toxin mediated cell ablation. Mice have no diphtheria toxin receptor, and are therefore insensitive to the toxin. In transgenic mice expressing the human DT-receptor, such as the Foxl1-hDTR BAC transgenic mice, hDTR-expressing cells are ablated by DT treatment.

FIG. 22: Strategy for the derivation of a Foxl1-CreERT2 BAC. Transgenic founders were identified by PCR with primers specific for the CreERT2 cDNA.

FIG. 23: Sonic hedgehog signaling cascade. The hedgehog receptor complex contains the signal transducing receptor smoothened and an inhibitory subunit, Patched (Ptc) that blocks smo activity. In the absence of hedgehog (hh) ligand. Gli proteins are processed to forms that actively repress hedgehog targets. Hh binding to Ptc releases Ptc from Smo, leading to Gli processing to activator forms. Gli1 and Ptc are also transcriptional targets of Hh signaling (adapted from McMahon, Cell, 2000, 100:185).

FIG. 24: Canonical Wnt/β-catenin signaling cascade. In the absence of wnt, intracellular levels off β-catenin are regulated by a complex of proteins including GSK3β (glycogen synthase kinase-3β), CK1 (casein kinase 1), and the scaffold proteins APC (adenomatous polyposis coli), Axin 1, and Axin 2 that serve to direct β-catenin to the ubiquitin-mediated degradation pathway. Wnt ligand engagement to the frizzled receptor in concert with coactivator receptors LRF (low-density lipoprotein receptor-related proteins) results in the inhibition of the degradation complex, stabilization of β-catenin and nuclear translocation where it binds to members of the TCF/LEF transcription factor family and activates gene transcription.

FIG. 25: Notch signaling cascade. Activation of Notch receptors by their ligands (e.g. Jagged) leads to proteolytic release of the notch intracellular domain (NICD). NICD then translocates to the nucleus where it binds to CSL/RBPjκ, resulting in recruitment of coactivators including Mastermind (Mam1) and p300 and activation of Notch target genes. In the absence of NICD binding, CSL/RBPjκ inhibits target genes by recruiting a repressor complex. This repressor complex is replaced by a coactivator complex that is recruited in response to NICD binding to CSL/RBPjκ. On the Internet at: (isrec.ch/research/groups/research_groups_detail_eid_{—}3263_lid_{—}2.htm)

FIG. 26: Increased necrosis in Foxl1 mutants. Bile duct ligation was performed and analyzed by trichrome staining two weeks later in Foxl1^{+/+} (left panel) and Foxl1^{-/-} livers. Necrosis (lighter parenchymal areas) is more extensive in Foxl1^{-/-} livers. 10× magnification.

FIG. 27: Cholangiocyte area is reduced in Foxl^{-/-} livers post-BDL. Representative liver sections from CK19 stained 14 day BDL Foxl1^{+/+} (left upper panel) and Foxl1^{-/-} animals (left lower panel). Quantification of CK19⁺/total area (right upper panel). **P-value=0.0001 relative to 14 day Foxl1^{-/-}BDL.

FIG. 28: Cyclin D1 activation is decreased in Foxl1 null cholangiocytes 5 days post-BDL. Cyclin D1 expression in cholangiocytes was quantified in 5 and 14 day post-BDL Foxl1^{+/+}, Foxl1^{-/-} and sham operated livers. Quantification of percent Cyclin D1 positive cholangiocyte in ten 20× fields at indicated time points post-BDL *P<0.05 relative to 5 day BDL Foxl1^{-/-}.

FIG. 29: Cholangiocyte proliferation is reduced in Foxl1 null livers following BDL. PCNA (red) and CK19 (green) staining in 5 day post-BDL Foxl1^{+/+} (A) and Foxl1^{-/-} (B) livers. Quantitation of percent PCNA⁺/CK19⁺ cells (C). *P-value <0.01 relative to 5 day Foxl1^{-/-}BDL. 40× magnification.

FIG. 30: Expression of Wnt3a and Wnt7b is decreased 5 days after BDL in Foxl1 null livers. Quantitative reverse transcription PCR for Wnt3a and 7b normalized to reference gene TBP. *P-value <0.05 relative to 5d Foxl1 null BDL **P-value <0.01 relative to 5d Foxl1 null BDL. #P-value <0.001 relative to 5d Foxl1^{+/+}BDL.

FIG. 31: Hepatocyte proliferation is delayed in Foxl1^{-/-} livers following cholestatic liver injury. Hepatocyte proliferation was quantified by Ki67 staining in livers 5 and 14 day post-BDL and sham operated Foxl1⁺⁺ and Foxl1^{-/-} livers. *P<0.05 relative to 5 day Foxl1^{-/-} BDL, **P=0.001 relative to Foxl1^{+/+} 14 day BDL, #P <0.05 relative to 14 day Foxl1^{+/+}BDL. N=4-7 samples for each time point.

FIG. 32: Notch ligand Jagged1 and transcriptional target Hey2 are induced enriched in the peri-portal region after BDL injury. Laser capture microdissection from portal tracts and adjacent parenchyma were obtained and RNA isolated for gene expression analysis. Quantitative reverse transcriptase PCR confirmed induction of Jagged1 (left panel) and Hey2 (right panel) following at indicated times post-BDL in portal and parenchymal LCM samples.

FIG. 33: Proposed mechanisms of progenitor cell proliferation and differentiation. Following liver injury, signals to portal fibroblasts lead to the synthesis and secretion of hedgehog ligands that activate expression of hedgehog targets required for progenitor cell self-renewal and proliferation including Shh and Ptc as well as Foxl1. Activation of Foxl1 in progenitor cells leads to increased expression of Wnt ligands and other β-catenin regulated proteins important for autocrine activation progenitor/cholangiocyte proliferation and paracrine activation of hepatocyte proliferation. Activation of the Notch signals directs differentiation of progenitors to the cholangiocyte lineage.

FIG. 34: Deletion of the hedgehog receptor smoothened in Foxl1⁺ progenitor cells is associated with increased acute and chronic injury two weeks after BDL. Liver sections from smo^{loxP/loxP} controls (A, C) and Foxl1Cre;smo^{loxP/loxP} mice (1B,D) two weeks after BDL. Panels A,B are stained with H&E. Blue areas stained with trichrome represent collagen (C, D). A and B 10×, C and D, 20× magnification. Arrow: area of hepatic necrosis. Arrowhead: area of collagen deposition (D).

FIG. 35: Mating scheme for the conditional gene ablation of smo in Foxl1⁺ hepatic progenitor cells. The Smo^{loxP/loxP};Rosa^{YFP/YFP} mice will be obtained from mating Smo^{loxP/+} Rosa^{YFP/+} heterozygotes. The Foxl1CreERT2;Smo^{loxP/+} mice will be obtained from mating Foxl1CreERT2 with Smo^{loxP/+} mice. All genotypes will be determined by PCR with gene specific primers.

FIG. 36: Mating scheme for obtaining Foxl1^{-/-};Foxl1CreERT2;RosaYFP/+ and Foxl1⁺/;Foxl1CreERT2; Rosa^{YFP/+} mice. Foxl1^{+/-};Rosa^{YFP/YFP} mice will be obtained from mating Foxl1^{+/-};Rosa^{YFP}/+ heterozygotes. Foxl1^{+/-};Foxl1CreERT2 mice will be obtained from mating Foxl1^{+/-};Foxl1CreERT2 heterozygotes.

### DETAILED DESCRIPTION OF THE INVENTION

Expression profiling studies have identified a subpopulation of hepatic progenitors that express markers consistent with a mixed epithelial/mesenchymal phenotype and that include members of the forkhead winged helix transcription factor family (Yovchev et al. (2008) Hepatology 47:636-647; Dan Y Y et al., (2006) Proc Natl Acad Sci USA 103:9912-9917). The forkhead winged helix factor, Foxl1 (Forkhead Box 11, formerly Fkh6), had previously been identified as a mesenchymal factor in the intestine with undetectable expression in the developing and adult liver (Kaestner K H, et al, (1997) Genes Dev 11:1583-1595). Hereinbelow, it is demonstrated that Foxl1 is expressed in rare cells in the normal liver and is dramatically induced in livers that have sustained injury. In a particular embodiment of the invention, adult hepatic stem cells express Foxl1 to detectable levels. In another embodiment, the adult hepatic stem cells express Foxl1 to levels greater than normal adult liver cells, particularly uninjured adult liver cells. It is also demonstrated hereinbelow that the early Foxl1-Cre lineage cells gives rise to both cholangiocytes and hepatocytes following liver injury and indicate the potential for progenitor-portal fibroblast cell interactions. Accordingly, Foxl1 is a marker of hepatic stem cells.

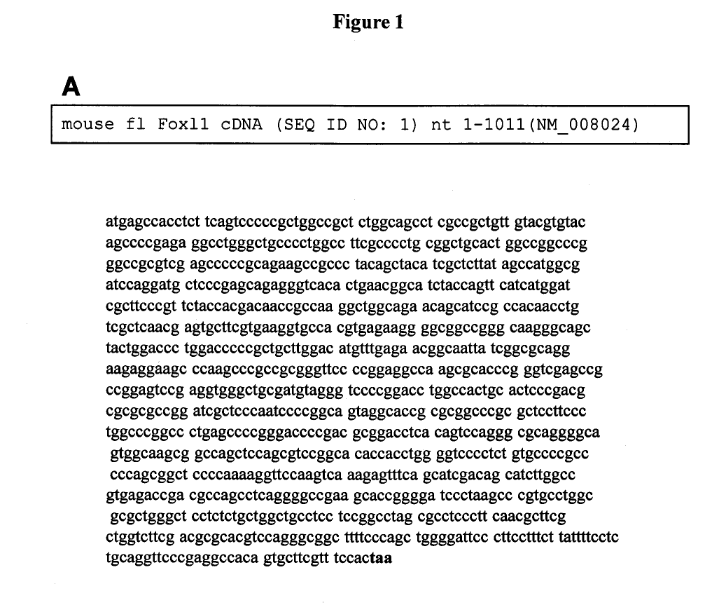

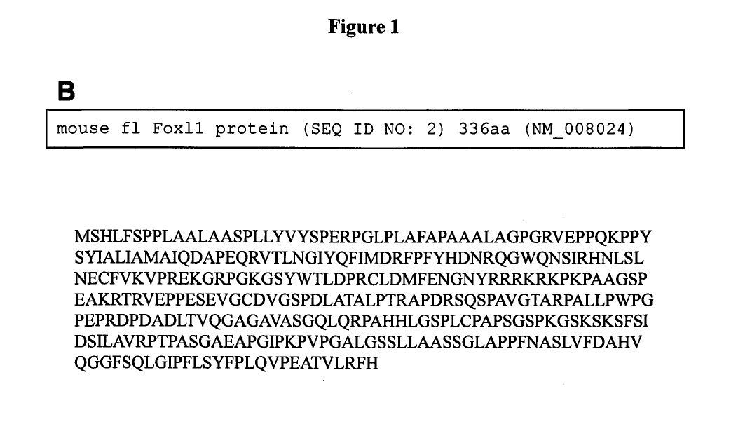

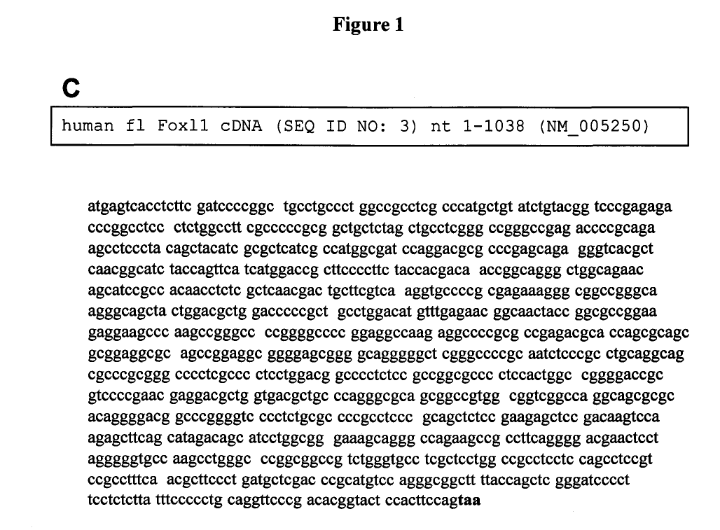

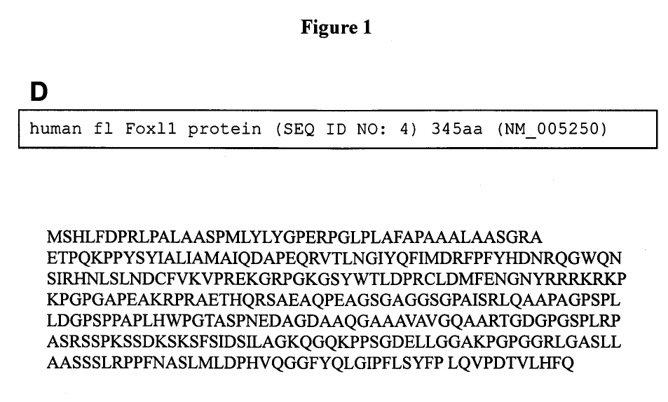

FIG. 1 (panels A-D) provides exemplary nucleic acid and amino acid sequences of mouse and human Foxl1. The instant invention encompasses variants of the amino acid sequence of Foxl1. For example, the amino acid sequence of mouse or human Foxl1 variants has 80%, 85%, 90%, 95%, 97%, or 99% identity with SEQ ID NOs: 2 or 4. Also encompassed by the instant invention are variants of the nucleic acid sequences Foxl1. For example, the nucleic acid sequence of mouse or human Foxl1 variants has 80%, 85%, 90%, 95%, 97%, or 99% identity with SEQ ID NOs: 1 or 3.

### I. DEFINITIONS

The following definitions are provided to facilitate an understanding of the present invention.

The phrase “Foxl1 activity” includes without limitation, modulation of biliary cell proliferation, modulation of hepatocyte proliferation, modulation of hedgehog signaling, modulation of notch signaling, modulation of liver cell necrosis and/or apoptosis, modulation of cyclin D activation and cholangiocyte proliferation. As noted herein, Foxl1 expression can be used to advantage to identify biopotential hepatic progenitor cells.

A “stem cell” as used herein is a undifferentiated cell which is capable of essentially unlimited propagation either in vivo or ex vivo and capable of differentiation to other cell types. This can be to certain differentiated, committed, immature, progenitor, or mature cell types present in the tissue from which it was isolated, or dramatically differentiated cell types, such as for example the erythrocytes and lymphocytes that derive from a common precursor cell, or even to cell types at any stage in a tissue completely different from the tissue from which the stem cell is obtained. For example, blood stem cells may become brain cells or liver cells, neural stem cells can become blood cells, such that stem cells are pluripotential, and given the appropriate signals from their environment, they can differentiate into any tissue in the body.

Stem cells express morphogenic or growth hormone receptors on the cell surface, and can sense, for example, injury-related factors then localize to and take residence at sites of tissue injury, or sense their local microenvironment and differentiate into the appropriate cell type.

The phrase “bipotential hepatic progenitor cell” refers to a pluripotent cell with the capacity to differentiate into a hepatocyte or a cholangiocyte.

“Essentially unlimited propagation” can be determined, for example, by the ability of an isolated stem cell to be propagated through at least 50, preferably 100, and even up to 200 or more cell divisions in a cell culture system. Stem cells can be “totipotent,” meaning that they can give rise to all the cells of an organism as for germ cells. Stem cells can also be “pluripotent,” meaning that they can give rise to many different cell types, but not all the cells of an organism. When a stem cell differentiates it generally gives rise to a more adult cell type, which may be a partially differentiated cell such as a progenitor cell, a differentiated cell, or a terminally differentiated cell. Stem cells can be highly motile.

The phrase “stimulate the growth, proliferation, differentiation and/or mobilization of a stem cell” as used herein means that the substance can stimulate or enhance the growth, proliferation, differentiation and/or mobilization of a stem cell as compared to the growth, proliferation, differentiation and/or mobilization of a stem cell in the absence of the substance.

“Isolating” a stem cell refers to the process of removing a stem cell from a tissue sample and separating away other cells which are not stem cells of the tissue. An isolated stem cell will be generally free from contamination by other cell types and will generally have the capability of propagation and differentiation to produce mature cells of the tissue from which it was isolated. However, when dealing with a collection of stem cells, e.g., a culture of stem cells, it is understood that it is practically impossible to obtain a collection of stem cells which is 100% pure. Therefore, an isolated stem cell can exist in the presence of a small fraction of other cell types which do not interfere with the utilization of the stem cell for analysis or production of other, differentiated cell types. Isolated stem cells will generally be at least 30%, 40%, 50%, 60%, 70%, 80%, 85%, 90%, 95%, 98%, or 99% pure. Preferably, isolated stem cells according to the invention will be at least 98% or at least 99% pure.

A stem cell is “expanded” when it is propagated in culture and gives rise by cell division to other stem cells and/or progenitor cells. Expansion of stem cells may occur spontaneously as stem cells proliferate in a culture or it may require certain growth conditions, such as a minimum cell density, cell confluence on the culture vessel surface, or the addition of chemical factors such as growth factors, differentiation factors, or signaling factors.

A stem cell, progenitor cell, or differentiated cell is “transplanted” or “introduced” into a mammal when it is transferred from a culture vessel into a patient. Transplantation, as used herein, can include the steps of isolating a stem cell according to the invention and transferring the stem cell into a mammal or a patient. Transplantation can involve transferring a stem cell into a mammal or a patient by injection of a cell suspension into the mammal or patient, surgical implantation of a cell mass into a tissue or organ of the mammal or patient, or perfusion of a tissue or organ with a cell suspension. The route of transferring the stem cell or transplantation, will be determined by the need for the cell to reside in a particular tissue or organ and by the ability of the cell to find and be retained by the desired target tissue or organ. In the case where a transplanted cell is to reside in a particular location, it can be surgically placed into a tissue or organ or simply injected into the bloodstream if the cell has the capability to migrate to the desired target organ.

Transplantation, as used herein, can include the steps of isolating a stem cell according to the invention, and culturing and transferring the stem cell into a mammal or a patient. Transplantation, as used herein, can include the steps of isolating a stem cell according to the invention, differentiating the stem cell, and transferring the stem cell into a mammal or a patient. Transplantation, as used herein, can include the steps of isolating a stem cell according to the invention, differentiating and expanding the stem cell and transferring the stem cell into a mammal or a patient.

A “transplant graft” as used herein refers to at least 10⁵ stem cells according to the invention and up to 10⁸ or 10⁹ stem cells.

An “immunosuppressive agent” is any agent that prevents, delays the occurrence of or reduces the intensity of an immune reaction against a foreign cell in a host, particularly a transplanted cell. Preferred are immunosuppressive agents which suppress cell-mediated immune responses against cells identified by the immune system as non-self. Examples of immunosuppressive agents include but are not limited to cyclosporin, cyclophosphamide, prednisone, dexamethasone, methotrexate, azathioprine, mycophenolate, thalidomide, FK-506, systemic steroids, as well as a broad range of antibodies, receptor agonists, receptor antagonists, and other such agents as known to one skilled in the art.

A “mitogen” is any agent that stimulates mitosis and cell proliferation of a cell to which the agent is applied.

A “differentiation factor” is any agent that causes a stem cell or progenitor cell to differentiate into another cell type. Differentiation is usually accomplished by altering the expression of one or more genes of the stem cell or progenitor cell and results in the cell altering its structure and function.

A “signaling factor” as used herein is an agent secreted by a cell which has an effect on the same or different cells. For example, a signaling factor can inhibit or induce the growth, proliferation, or differentiation of itself, neighboring cells, or cells at distant locations in the organism. Signaling factors can, for example, transmit positional information in a tissue, mediate pattern formation, or affect the size, shape and function of various anatomical structures.

As used herein, a mammal refers to any mammal including but not limited to human, mouse, rat, sheep, monkey, goat, rabbit, hamster, horse, cow or pig.

A “non-human mammal”, as used herein, refers to any mammal that is not a human.

As used herein, “allogeneic” refers to genetically different members of the same species.

As used herein, “isogeneic” refers to of an identical genetic constitution.

As used herein, “xenogeneic” refers to members of a different species.

As used herein, “culturing” refers to propagating or nurturing a cell, collection of cells, tissue, or organ, by incubating for a period of time in an environment and under conditions which support cell viability or propagation. Culturing can include one or more of the steps of expanding and proliferating a cell, collection of cells, tissue, or organ according to the invention.

For purposes of the invention, “Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. Alternatively, this term may refer to a DNA that has been sufficiently separated from (e.g., substantially free of) other cellular components with which it would naturally be associated. “Isolated” is not meant to exclude artificial or synthetic mixtures with other compounds or materials, or the presence of impurities that do not interfere with the fundamental activity, and that may be present, for example, due to incomplete purification. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

With respect to single-stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (see Sambrook et al. (2001) Molecular Cloning. A Laboratory Manual, Cold Spring Harbor, N.Y.: Cold Spring Harbor Laboratory Press):

Tₘ=81.5° C.+16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. Depending upon the specific sequence involved, the Tₘ of a DNA duplex decreases by 0.5-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high-stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The present invention also includes active portions, fragments, derivatives and functional or non-functional mimetics of the polypeptides of the invention. “Peptide” and “polypeptide” are used interchangeably herein and refer to a compound made up of a chain of amino acid residues linked by peptide bonds. An “active portion” of a polypeptide means a peptide that is less than the full length polypeptide, but which retains measurable biological activity and retains biological detection.

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds.

According to the present invention, an isolated, or biologically pure molecule or cell is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

A “sample” or “patient sample” or “biological sample” generally refers to a sample which may be tested for a particular molecule, preferably polynucleotide, polypeptide, or antibody. Samples may include but are not limited to cells, tissue, and body fluids, including blood, serum, plasma, cerebral-spinal fluid, urine, saliva, pleural fluid and the like. A “test subject” includes, but is not limited to animals, including mammalian species such as murine, porcine, ovine, bovine, canine, feline, equine, human, and other primates.

A “fragment” or “portion” of a polypeptide means a stretch of amino acid residues of at least about five to seven contiguous amino acids, often at least about seven to nine contiguous amino acids, typically at least about nine to fifteen contiguous amino acids and, most preferably, at least about fourteen or more contiguous amino acids.

A “derivative” of a polypeptide or a fragment thereof means a polypeptide modified by varying the amino acid sequence of the protein, e.g. by manipulation of the nucleic acid encoding the protein or by altering the protein itself. Such derivatives of the natural amino acid sequence may involve insertion, addition, deletion or substitution of one or more amino acids, and may or may not alter the essential activity of original the polypeptide.

As mentioned above, a polypeptide or protein of the invention includes any analogue, fragment, derivative or mutant which is derived from a polypeptide and which retains at least one property or other characteristic of the polypeptide. Different “variants” of the polypeptide exist in nature. These variants may be alleles characterized by differences in the nucleotide sequences of the gene coding for the protein, or may involve different RNA processing or post translational modifications. The skilled person can produce variants having single or multiple amino acid substitutions, deletions, additions or replacements. These variants may include inter alia: (a) variants in which one or more amino acids residues are substituted with conservative or non conservative amino acids, (b) variants in which one or more amino acids are added to the polypeptide, (c) variants in which one or more amino acids include a substituent group, and (d) variants in which the polypeptide is fused with another peptide or polypeptide such as a fusion partner, a protein tag or other chemical moiety, that may confer useful properties to the polypeptide, such as, for example, an epitope for an antibody, a polyhistidine sequence, a biotin moiety and the like. Other polypeptides of the invention include variants in which amino acid residues from one species are substituted for the corresponding residue in another species, either at the conserved or non conserved positions. In another embodiment, amino acid residues at non conserved positions are substituted with conservative or non conservative residues. The techniques for obtaining these variants, including genetic (suppressions, deletions, mutations, etc.), chemical, and enzymatic techniques are known to the person having ordinary skill in the art.

To the extent such allelic variations, analogues, fragments, derivatives, mutants, and modifications, including alternative nucleic acid processing forms and alternative post translational modification forms result in derivatives of the polypeptide that retain any of the biological properties of the polypeptide, they are included within the scope of this invention.

The term “functional” as used herein implies that the nucleic or amino acid sequence is functional for the recited assay or purpose.

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide can depend on various factors and on the particular application and use of the oligonucleotide.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “gene” refers to a nucleic acid comprising an open reading frame encoding a polypeptide, including both exon and (optionally) intron sequences. The nucleic acid may also optionally include non coding sequences such as promoter or enhancer sequences. The term “intron” refers to a DNA sequence present in a given gene that is not translated into protein and is generally found between exons.

The term “promoter” or “promoter region” generally refers to the transcriptional regulatory regions of a gene. The “promoter region” may be found at the 5′ or 3′ side of the coding region, or within the coding region, or within introns. Typically, the “promoter region” is a nucleic acid sequence which is usually found upstream (5′) to a coding sequence and which directs transcription of the nucleic acid sequence into mRNA. The “promoter region” typically provides a recognition site for RNA polymerase and the other factors necessary for proper initiation of transcription.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and method of use. For example, depending on the complexity of the target sequence, the oligonucleotide probe typically contains about 10-50 or more nucleotides, more preferably, about 15-25 nucleotides.

The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

The terms “recombinant organism”, or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organisms.

A “clone” or “clonal cell population” is a population of cells derived from a single cell or common ancestor by mitosis. A “cell line” is a clone of a primary cell or cell population that is capable of stable growth in vitro for many generations. In a particular embodiment of the invention, the hepatic progenitor cells described herein differentiate into liver cells.

An “antibody” or “antibody molecule” is any immunoglobulin (e.g., IgG, IgM, IgA, IgE, IgD, etc.), including antibodies and fragments thereof, that binds to a specific antigen. The term includes polyclonal, monoclonal, chimeric, and bispecific antibodies. As used herein, antibody or antibody molecule includes recombinantly generated intact immunoglobulin molecules and immunologically active portions of an immunoglobulin molecule such as, without limitation: Fab, Fab′, F(ab′)₂, F(v), scFv, scFv₂, scFv-Fc, minibody, diabody, tetrabody, single variable domain (e.g., variable heavy domain, variable light domain), bispecific, Affibody® molecules (Affibody, Bromma, Sweden), and peptabodies (Terskikh et al. (1997) PNAS 94:1663-1668). An antibody can be obtained from any source such as, without limitation, humans, rodents, non-human primates, lagomorphs, caprines, bovines, equines, and ovines. Methods for recombinantly producing antibodies are well-known in the art. For example, commercial vectors comprising constant genes to make IgGs from scFvs are provided by Lonza Biologics (Slough, United Kingdom). As used herein, the term “monoclonal antibody” or “mAb” refers to any essentially homogeneous antibody or antigen-binding region thereof that is reactive with, preferably specifically reactive with, a single epitope (antigenic determinant). The term “monoclonal antibody” may refer to generally homogeneous antibodies that are native, modified, or synthetic, and can include hybrid or chimeric antibodies. Typically, monoclonal antibodies are derived from a single clone of B lymphocytes (i.e., B cells). Monoclonal antibodies may be produced by methods known in the art.

A “polyclonal antibody” is a group of heterogeneous antibodies that recognize epitopes or antigenic determinants present on a protein. Typically, polyclonal antibodies originate from many different clones of antibody-producing cells.

The phrase “specifically recognizes”, as used herein, refers to the binding affinity demonstrated by an antibody for its cognate antigen. The skilled artisan is aware of the many methods available to measure the binding affinity of an antibody for an antigen (see, e.g., Antibodies, A Laboratory Manual, (1988) Cold Spring Harbor Publications, New York). Preferably, the antibody demonstrates binding affinity for the protein(s) of interest to the exclusion of other proteins.

The term “detectable label” is used to herein to refer to any substance whose detection or measurement, either directly or indirectly, by physical or chemical means, is indicative of the presence of the target bioentity. Representative examples of useful detectable labels, include, but are not limited to the following: molecules or ions directly or indirectly detectable based on light absorbance, fluorescence, reflectance, light scatter, phosphorescence, or luminescence properties; molecules or ions detectable by their radioactive properties; molecules or ions detectable by their nuclear magnetic resonance or paramagnetic properties. Included among the group of molecules indirectly detectable based on light absorbance or fluorescence, for example, are various enzymes which cause appropriate substrates to convert, e.g., from non-light absorbing to light absorbing molecules, or from non-fluorescent to fluorescent molecules. Further examples of detectable labels include, without limitation: biotin, avidin, fluorescent compound, a radioisotope, and an enzyme.

The terms “percent similarity”, “percent identity” and “percent homology” when referring to a particular sequence are used as set forth in the University of Wisconsin GCG software program.

The term “delivery” as used herein refers to the introduction of foreign molecule (i.e., protein containing nanoparticle) into cells. The term “administration” as used herein means the introduction of a foreign molecule into a cell. The term is intended to be synonymous with the term “delivery”. Administration also refers to screening assays of the invention (e.g., routes of administration such as, without limitation, intravenous, intra-arterial, intramuscular, subcutaneous, intrasynovial, infusion, sublingual, transdermal, oral, or topical).

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxilliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E.W. Martin (Mack Publishing Co., Easton, Pa.); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3.sup.rd Ed.), American Pharmaceutical Association, Washington, 1999.

The term “kit” refers to a combination of reagents and other materials.

### II. ANTIBODIES

For the production of antibodies, various host animals may be immunized by injection with the protein of interest, e.g., Foxl1 (e.g., SEQ ID NOs: 2 or 4) and/or fragments and variants thereof. Such host animals include, without limitation, rabbits, mice, goats, and rats. Those of skill in the art are well apprised of the various adjuvants which may be employed to increase antibody titer obtained from the host. Common adjuvants include, without limitation, Freund's adjuvant, Ribi adjuvant, and the like. Other methods of increasing antibody production are known to those skilled in the art in supplement or in lieu of the use of adjuvants.

Methods for obtaining polyclonal sera (antibodies) and monoclonal antibodies are well known in the art (see, in general, Ausubel et al. (eds.), Current Protocols in Molecular Biology, John Wiley & Sons, Inc., New York, 1994); Coligan, Current Protocols in Immunology, Wiley/Greene, New York (1991); and Harlow and Lane (1988) Antibodies, A Laboratory Manual, Cold Spring Harbor Publications, New York). Other techniques are taught by Kohler and Milstein (Nature (1975) 256:495-497); Mayer and Walker (Immunochemical Methods in Cell and Molecular Biology, (1987) Academic Press, London); U.S. Pat. No. 4,376,110; Kosbor et al. (Immunol. Today (1983) 4:72); Cole et al. (Proc. Natl. Acad. Sci. USA (1983) 80:2026-2030); Cole et al. (Monoclonal Antibodies And Cancer Therapy (1985) Alan R. Liss, Inc., pp. 77-96).

The antibody molecules of the invention may be prepared using any method known in the art. Antibodies may be prepared by chemical cross-linking, chimeric antibody technology, hybrid hybridoma techniques, and by expression of recombinant antibody fragments expressed in host cells, such as, without limitation, bacteria or yeast cells.

The antibody molecules may also be produced by expression of recombinant antibody fragments in host cells. The resulting antibody molecules may then be isolated and purified from the expression system. The antibodies optionally comprise a purification tag to facilitate purification of the antibody.

The purity of the antibody molecules of the invention may be assessed using standard methods known to those of skill in the art, including, but not limited to, ELISA, immunohistochemistry, ion-exchange chromatography, affinity chromatography, immobilized metal affinity chromatography (IMAC), size exclusion chromatography, polyacrylamide gel electrophoresis (PAGE), western blotting, surface plasmon resonance and mass spectroscopy. In one embodiment of the invention, the antibody binds immunospecifically to a region C-terminal to amino acid residue 147 of SEQ ID NOs: 2 and 4.

### III. TRANSGENIC ANIMALS

The Foxl1-Cre mouse described herein represents a unique tool that allows for the isolation of progenitor cells that can be used in co-culture experiments to investigate mechanisms of progenitor cell differentiation and self-renewal. These mice can also be utilized to specifically ablate any gene of interest in the progenitor cell lineage.

More specifically, based on the data hereinbelow, it is evident that Foxl1-Cre is expressed in hepatic progenitors that differentiate into liver cells, such as biliary epithelial cells or hepatocytes, following injury (e.g., bile duct ligation). Previous studies have demonstrated that N-acetylparaminophen (APAP) treatment, 3,5-diethoxycarbonyl-1,4-dihydrocollidine (DDC)-supplemented, or choline-deficient ethionine-supplemented (CDE) diets induce oval cell proliferation in mice (29). However, differences in injury type may influence the progenitor cell niche and microenvironment. Foxl1-Cre mice can be crossed with a dual reporter Z/EG mouse line (35) in which activation of Foxl1-Cre can result in the excision of a lacZ spacer and activation of eGFP expression. These calls can be tested in the three aforementioned paradigms of liver injury. The model with the most robust and widespread activation of Foxl1 can be employed for progenitor cell isolation experiments.

While GFP is exemplified, other fluorescent markers or other detectable markers may be used (i.e., YFP). One advantage of fluorescent markers, such as eGFP is that it can allow for isolation of progenitor cells and their descendants by collagenase perfusion and FACS sorting in future studies. Isolated cells can then be analyzed by expression profiling to determine if there are differences in progenitor cells based on the experimental method used, and to identify specific cell surface proteins that might be employed for the isolation of the equivalent cell populations from human liver.

Experimentally, Foxl1-Cre mice can be crossed with Z/EG mice. 4-6 week old male Foxl1-Cre;Z/EG can undergo the following treatments, optimized to induce maximal hepatic progenitor cell proliferation (29). Groups of 5 mice can be used for each treatment time point. DDC diet: standard chow supplemented with 0.1% DDC (Sigma-Aldrich) for 6 weeks. CDE: choline-deficient diet (ICN) supplemented with 0.165% DL-ethionine for 4 weeks. APAP: Mice can be fasted for 8 hours prior to intraperitoneal injection with APAP 500 mg/kg dose (Sigma-Aldrich) and livers harvested 48 hours after injection. A portion of each liver can be fixed in neutral buffered formalin and stained with hematoxylin and eosin for histologic examination. For visualization of eGFP, tissue sections can be embedded in OCT, frozen at −70° C., cryo-sectioned and mounted for visualization of fluorescence. IP lab software can be used to quantify the numbers of eGFP positive cells. To isolate eGFP cells for expression profiling and marker analysis, sequential collagenase and pronase digestion can be utilized with established methods for the isolation of oval cell enriched non-parenchymal cell fractions (36). Cells can be analyzed by flow cytometry using 480 nm wavelength for excitation and 530 nm wavelength for emission to sort for eGFP fluorescence (35). This cell population can be characterized more extensively using profiling of gene expression and analysis of markers associated with progenitor cell populations including stem cell factor, CK19, CK7, DIWPref-I, ABCG2, c-kit, thy-I, CD34, OV6, NCAM, AbcG2, EpCAM, and alpha fetoprotein. RosaYFP mice can also be used for isolation experiments described herein.

It has also been demonstrated that Foxl1-Cre is activated only in biliary progenitors at early time points post-BDL (3-7 days post-BDL) whereas Foxl1-Cre/HNF-4α co-localization was also detected in scattered hepatocytes near the portal tracts at later time points (2 weeks post-BDL). These observations indicate that Foxl1-Cre is activated in bipotential progenitor cells that subsequently differentiate towards biliary and hepatocytic lineages. To further study the progenitor-descendant relationship between the early Foxl1 positive cells in the ductular reaction and late hepatocytes, an inducible Cre can be used. For example, a transgenic mouse expressing the estrogen-inducible CreERT2 protein to label Foxl1 expressing cells can be made. Because CreERT2 is only activated transiently, any eGFP-positive hepatocyte present in Foxl1 CreERT2; Z/EG mice two weeks after BDL must have come from an early, HNF4α and CK19 negative, but Foxl1⁺ progenitor.

A novel transgenic mouse that expresses an inducible Cre (e.g., an estrogen-inducible Cre (ERT2)) under the control of the Foxl1 promoter is encompassed by the invention. Methods for making such mice are generally described in (39). The Foxl1 coding region can be replaced with the CreERT2 cassette using BAC recombineering as described previously (40). Transgenic founders can be identified by PCR of tail DNA for the CreERT2 sequence, and bred to Z/EG reporter mice. Bi-transgenic Foxl1 CreERT2; Z/EG mice can be tested for efficient Cre activation by i.p. injection of 2 mg tamoxifen, and visualization of eGFP in frozen sections of the GI tract, where Foxl1 is expressed constitutively (40). Founders that faithfully recapitulate Foxl1 expression can be expanded, and Foxl1CreERT2; Z/EG mice subjected to BDL and DDC, CDE and APAP as described above. 24 hours after BDL, the mice can receive a single 2 mg tamoxifen injection to activate the Foxl1-CreERT2. Control mice can receive a vehicle (sunflower oil) injection. Mice can be sacrificed 2 weeks after BDL and eGFP expressing cells detected as described hereinabove. Thus, eGFP expressing cells that co-label with markers of biliary (CK19) and hepatocyte differentiation (HNF-4α) can be detected to confirm that Foxl1 expressing cells represent bipotential hepatic progenitor cells.

Furthermore, Foxl1 mutant mice exhibit reduced biliary proliferation and increased necrosis in the bile duct ligation model of cholestatic liver injury. These findings indicate that Foxl1 activity in hepatic progenitors is required for cholangiocyte differentiation, proliferation and liver repair. Ablation of the entire Foxl1 progenitor lineage can be associated with more severe hepatic necrosis than in Foxl1 mutant BDL livers.

To achieve cellular ablation, a diphtheria toxin based system will be utilized. By way of background, mice are normally insensitive to diphtheria toxin (DT). Therefore, only cells in which the human diphtheria toxin receptor (hDTR) is expressed can be ablated following hDTR injection. Foxl1-hDTR BAC transgenic mice direct expression of the hDTR to the Foxl1 progenitor lineage, allowing for their ablation during the regenerative response.

Thus, in a particular embodiment of the invention, Foxl1-hDTR mice are provided. The mice can undergo either BDL, or be treated with DDC, CDE or APAP, as described above. DT can be injected daily beginning on the day of onset of the experimental treatment. Control cohorts can include Foxl1-hDTR mice treated with DT and sham BDL-operated Foxl1-hDTR mice treated with DT. As a result, ablation of Foxl1 expressing cells should be associated with increased liver injury, manifested by decreased biliary proliferation and increased hepatic necrosis in the BDL model and reduced oval cell proliferation in the DDC, CDE and APAP groups.

### IV. METHODS OF IDENTIFYING ADULT HEPATIC PROGENITOR CELLS

Also encompassed in the scope of the present invention are methods of identifying adult bipotential hepatic progenitor cells by obtaining a sample of hepatic cells from a subject, contacting the cells obtained with a biomolecule that specifically recognizes SEQ ID NOs: 1 or 3, or proteins encoded thereby, and assessing the cells for the presence of Foxl1. The presence of Foxl1 in the cells indicates that the cells are adult hepatic stem cells. Following identification, the adult hepatic stem cells identified by the biomolecule can be isolated by known methods, preferably by FACS. Biomolecules for identification are any composition of matter that recognizes Foxl1 to the exclusion of other nucleic acids or proteins. A Foxl1 biomolecule that specifically recognizes Foxl1 can be, for example, an oligonucleotide (e.g., probe or primer), a nucleic acid, a polypeptide, a peptide, or an antibody which recognize nucleic acids encoded by SEQ ID NOs: 1 or 3, or polypeptides encoded by SEQ ID NOs: 2 or 4. Preferably the biomolecule is an antibody or fragment thereof specific for SEQ ID NOs: 2 or 4. As mentioned, such antibodies or fragments thereof are useful as probes for detecting and isolating Foxl1 expressing adult hepatic stem cells.

In addition to Foxl1 expression, other markers that co-segregate with Foxl1 can be used in sorting and isolation applications. For example cell surface markers or markers associated with progenitor cell populations including stem cell factor, CK19, CK7, DIWPref-I, ABCG2, c-kit, thy-I, CD34, OV6, NCAM, EpCAM, and alpha fetoprotein.

### V. METHODS OF TREATMENT

Many disorders of the liver are associated with damage to hepatic cells. Accordingly, when regeneration of damaged hepatic cells is desired, the compositions and methods of the invention can be used to increase the population of hepatic stem cells at the site of damage thereby facilitating liver tissue repair. One way that this can be accomplished is by identifying cells expressing Foxl1, isolating the cells, and delivering the isolated cells back into a patient for therapy. In another embodiment, Foxl1 expressing cells can be identified from a compatible donor, isolated, and provided to a distinct recipient in need of therapy. Therapy may be required to treat a variety of disorders including hepatocellular cancers such as hepatocellular carcinoma, cholangiocarcinoma; biliary diseases including biliary atresia, primary biliary cirrhosis, primary sclerosing cholangitis; cirrhosis due to a variety of primary etiologies including autoimmune hepatitis, viral hepatitis B, viral hepatitis C, nonalcoholic fatty liver disease, hemochromatosis, alpha-1-antitrypsin deficiency, Wilson's Disease; fulminant liver failure due acute hepatitis, drug toxicity, Wilson's Disease, ischemia, inherited and acquired metabolic diseases including tyrosinemia, glycogen storage diseases, and disorders of bilirubin metabolism.

It is clear from the foregoing that Foxl1 can be used as a marker for hepatic stem or progenitor cells. Delivery of the isolated cells to a patient can be accomplished by any known means in the art. One skilled in the art appreciates that a plurality of isolated hepatic stem cells can be administered to a subject by various routes including, for example, parenterally, such as intravenously (i.v.), intramuscularly, intrasplenically, subcutaneously, intraorbitally, intranasally, intracapsularly, intraperitoneally (i.p.), intracisternally, or intra-articularly. The isolated hepatic stem cells of the invention can also be delivered directly to the site of damage and/or the surrounding tissue.

In a similar fashion, the invention allows the introduction of any genetic sequence selectively into hepatic progenitors including siRNAs, microRNA, or nucleic acid sequences encoding a protein of interest or fragments of such proteins. When the cells of the invention are genetically modified by the introduction of a heterologous nucleic acid coding sequence, they can be used in methods of gene therapy. For example, a progenitor cell of the invention may be modified, then cultured as described herein prior to re-implantation of cells (either progenitor cells or differentiated cells derived therefrom). Such ex vivo transformation procedures have safety advantages because the cells can be examined ex vivo to confirm that, for example, the transgene has not been incorporated close to an oncogene, thus rendering the transformed cell oncogenic. Methods of transformation known in the art may be used. In principle, any gene can be introduced in this manner. Generally, the gene will be one that complements a deficiency in the patient's own liver tissue.

The total treatment dose can be administered to a subject as a single dose or can be administered using a fractionated treatment protocol, in which multiple doses are administered over a more prolonged period of time, for example, over the period of a day to allow administration of a daily dosage or over a longer period of time to administer a dose over a desired period of time.

Furthermore, in the normal, uninjured liver, transplanted hepatocytes or bone marrow derived cells only rarely engraft in the liver. The success of engraftment is greatly enhanced when the transplanted cells have a dramatic growth advantage, such as in the fumarylacetoacetate hydrolase (FAH)-deficient mouse (37, 38). If the Foxl1-positive progenitor lineage truly has a higher regenerative potential, then much fewer transplanted cells should be required to restore liver homeostasis in the FAH-deficient model than when mature hepatocytes are used. This can be accomplished by establishing the FAH-deficient model, and the cell sorting of Foxl1-positive cells from Foxl1-Cre; Z/EG mice.

In a particular embodiment, donor mice (Foxl1-Cre;Z/EG strain) can receive DDC-supplemented diet for 4 weeks to induce oval cell proliferation (36). Nonparenchymal cells can be isolated using sequential collagenase and pronase digestion using published methods (36). Previous methods have utilized cell size as the principle criterion for enrichment of oval cells. This method has been associated with some degree of hepatocyte contamination (36). FACS sorting for GFP expression can provide a more homogeneous population of hepatic progenitors that can minimize hepatocyte contamination as Foxl1 is not expressed in hepatocytes. In prior engraftment experiments, 5×10⁵ mature hepatocytes (26) or 5×10⁵ sorted oval cells were necessary to restore liver homeostasis. Procedures can begin with 5×10⁵ number of Foxl1+ progenitors, and also test 2.5×10⁵ and 1.25×5×10⁵ cells.

5×10⁵ cells can be transplanted into FAH^{-/-} recipients that have been maintained on NTBC (2-(2-nitro-4-3 trifluoro-methylbenzoyl)-I,3-cyclohexanedione) in the drinking water until time of transplantation (36). NTBC treatment prevents the accumulation of pathogenic fumarylacetoacetate in FAH^{-/-} mice. If necessary, GFP sorted cells can be pooled from multiple animals to achieve sufficient numbers of cells for transplantation. Cells can be injected intrasplenically and NTBC withdrawn at the time of transplantation to allow for repopulation of the liver by the donor cells. Animals can be monitored for 6 weeks at which time livers can be harvested and analyzed for donor cell engraftment. Histological sections can be examined for eGFP fluorescence and the eGFP positive area quantified to determine the contribution of Foxl1 expressing progenitor cells for repopulation of the FAH^{-/-} liver.

As mentioned previously, a preferred embodiment of the invention comprises delivery of isolated hepatic stem cells to a patient in need thereof. Formulation, dosages and treatment schedules have also been described herein and are known to skilled artisans. Phase I clinical trials can be designed to assess the safety, tolerability, pharmacokinetics, and pharmacodynamics of the hepatic stem cells of the invention. These trials may be conducted in an inpatient clinic, where the subject suffering from a disease can be observed by full-time medical staff. After the initial safety of the cell-based therapy has been performed, Phase II trails can assess clinical efficacy of the therapy; as well as to continue Phase I assessments in a larger group of volunteers and patients. Subsequently, Phase III studies on large patient groups entails definitive assessment of the efficacy of hepatic stem cell delivery for a disease or disorder in comparison with current treatments. Finally, Phase IV trials involving the post-launch safety surveillance and ongoing technical support for cell transplantation therapy could be completed.

Cells of the invention may be used to repopulate the liver. Such cells may be autologous, i.e. derived from the patient's own liver (or that of an identical sibling), in which case no issues of tissue rejection arise. Alternatively, they may be allogeneic, i.e. derived from the liver of another individual. In the latter case, the cells will typically be derived from the liver of a donor individual with the same blood group as the recipient patient and anti-rejection drugs may be needed.

Such an approach could be used instead of or alongside a conventional liver transplant. The cells may be the patient's own cells, removed, cultured and replaced, or they may be cells from another individual, as discussed above. For example, they might be the patient's own cells in a chronic condition where there is time to remove and culture them without risking the patient's life (and such an approach may also be used in conjunction with liver dialysis). In acute cases, pre-cultured, non-patient cells may be preferred.

### VI. KITS AND ARTICLES OF MANUFACTURE

Any of the aforementioned compositions or methods can be incorporated into a kit which may contain at least one Foxl1 detection biomolecule, for example, an oligonucleotide (e.g., probe or primer), a polypeptide, a peptide, an antibody. The biomolecule may be detectably labeled. The kit can also contain a solid support (e.g., filters and cartridges), a pharmaceutically acceptable carrier, a physiologically acceptable carrier, instructions for use, negative and positive control samples, a container, a vessel for administration, or any combination thereof.

It is an additional feature of the invention to provide molding and cell matrix materials (i.e., a biocompatible polymeric matrix) to facilitate hepatic regeneration with the isolated cells of the invention. The biocompatible cell carrier or matrix may be a shaped construct, structure, or 3-dimensional scaffold. Examples of biocompatible carrier material includes alginate, agarose, fibrin, collagen, chitosan, gelatin, elastin, and combinations thereof. In one embodiment, the biocompatible cell carrier is biodegradable or bioresorbable. Examples of biodegradable matrix material may include, but not limited to, polymers or copolymers of lactide, glycolide, caprolactone, polydioxanone, and trimethylene carbonate. Examples of biodegradable matrix material may also include polyorthoesters and polyethylene oxide. The biocompatible polymeric matrix can also be formed from a material selected from the group of materials consisting of cellulose ether, cellulose, cellulosic ester, fluorinated polyethylene, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, copolymers thereof, and physical blends thereof.

The following examples illustrate certain embodiments of the invention. They are not intended to limit the scope of the invention in any way.

### Example 1

The following materials and methods are provided to facilitate practice of the instant invention. They are particularly relevant to Example 1.

### Mice and Experimental Protocols:

For lineage tracing studies, Foxl1-Cre mice were crossed to Rosa26R lacZ reporter mice (Soriano P., Nat Genet 1999; 21:70-71) and subjected to bile duct ligation (BDL). Foxl1-Cre-negative or sham-operated mice were used as controls. Animals (10 to 12 weeks old) were anesthetized with (2.5% V/V) vaporized isofluorane. A midline laparotomy was performed and the common bile duct ligated twice with 4.0 silk suture. Sham animals underwent a similar laparotomy, after which the common bile duct was exposed and manipulated without placement of ligatures. Mice received a subcutaneous injection of buprenorphine at 0.5 mg/kg immediately after surgery, were placed on a warming pad and allowed to recover. All protocols were approved by the IACUC of the University of Pennsylvania. At times of sacrifice, livers were rinsed in PBS and placed in 4% PFA for 45 minutes, rinsed in PBS and cryoprotected in 30% sucrose/PBS overnight at 4° C.

### Special Diets:

Mice were fed a diet containing 3,5-diethoxycarbonyl-1,4-dihydrocollidine (DDC) (Sigma-Aldrich, St. Louis, Mo.) (0.1% wt/wt) in #5015, PMI Mouse diet (Harlan Teklad, Madison, Wis.) for 3, 7, 14 or 21 days. Other mice were fed a choline deficient diet for five weeks (Harlan-Teklad, Diet TD88052) with drinking water supplemented with 0.165% ethionine (Sigma, St. Louis, Mo.). Livers were harvested and processed as described above.

### RNA Isolation and Quantitative Real-Time PCR:

Total RNA was extracted from liver samples using the Totally RNA Kit (Ambion, Applied Biosystems, Foster, City, Calif.). Liver RNA was reverse transcribed using oligo dT priming and SuperScript II reverse transcriptase (Invitrogen, Carlsbad, Calif.). PCR reactions were performed using SyBr Green QPCR Master Mix (Invitrogen, Carlsbad, Calif.) on an Mx3000 PCR cycler (Stratagene, Agilent Technologies, Santa Clara, Calif.). Reactions were performed in triplicate with reference dye normalization, and median C_{T} values were used in the analyses. Primers sequences were as follows: Foxl1 (F): TGCCGCATTCCACAGCATAGTC (SEQ ID NO: 5); Foxl1(R): CAAAGTGAGTTCCAGGACAGCCAG (SEQ ID NO: 6). CK19: (F): CCAGGAAGCCCACTACAACAAT (SEQ ID NO: 7); CK19 (R): TCGAGGGAGGGGTTAGAGTAAA (SEQ ID NO: 8). TATA binding protein (TBP) (F): CCCCTTGTACCCTTCACCAAT (SEQ ID NO: 9); TBP (R): GAAGCTGCGGTACAATTCCAG (SEQ ID NO: 10).

### Whole Mount Embryo β-Galactosidase Staining:

Embryos (12.5 dpc) were dissected in ice-cold PBS and fixed in 0.2% glutaraldehyde, 1% formaldehyde, 2 mM MgCl₂, 5 mM EGTA and 0.2% NP-40 for 1 h. Embryos were rinsed in PBS and incubated in X-gal staining solution overnight (Sanes J R, et al., (1986) Embo J 5:3133-3142). Embryos were rinsed in PBS and post-fixed in 4% PFA overnight at 4° C. Embryos were transferred to PBS, dehydrated through a graded PBS/methanol series, cleared in a 1:1 solution of benzyl alcohol:benzyl benzoate for 1 h, and imaged using a LeicaM212 microscope. For sectioning, embryos were re-hydrated using the reverse graded PBS/methanol series, rinsed in PBS, and incubated in 30% sucrose in PBS overnight at 4° C. Embryos were equilibrated in OCT for 60 minutes, oriented in fresh OCT and placed on dry ice to freeze. Cryo-sections were washed for 5 minutes each in a sequence of PBS, 4% PFA and then water. The slides were stained with Kernechtrot nuclear fast red (Poly Scientific, Bay Shore, N.Y.) 0.1% for 30 seconds and then sealed with crystal mount (Electron Microscopy Sciences, Hatfield, Pa.) to dry overnight. Cover slips were mounted on the slides using Histomount (Zymed, Invitrogen, Carlesbad, Calif.). Sections were imaged on a LeicaDMRE microscope.

### Histology and Immunohistochemistry:

10 μm cryosections were cut, warmed to room temperature and air dried for 5 minutes. Sections were incubated with X-gal (β-gal) staining solution (Sanes J R, et al., supra) at 37° C. for 3-5 hours. Sections were post-fixed for 5 minutes in 4% PFA, washed briefly in PBS, and mounted in aqueous mounting media (Kirkegaard, Gaithersburg, Md.).

For antibody co-localization studies for CK19, HNF-4α and Ki-67, β-gal stained sections were fixed for 5 minutes in 4% PFA. Antigen retrieval was performed by microwaving slides in 10 mM citric acid monohydrate buffer pH 6.0 for 6 minutes. The Vector ABC Elite kit detection method was used according to manufacturer's instructions (Vector Laboratories, Burlingame, Calif.). The primary antibodies were diluted in PBT (1×PBS, 0.1% BSA, 0.2% Triton-X) as follows: CK19 (Hybridoma Dev) 1:20; HNF-4α (Santa Cruz Biotechnology, Santa Cruz, Calif., sc-8947) 1:1000; Ki-67 (Vector Laboratories, Burlingame, Calif., VP-RM04) 1:1500 and incubated with sections overnight at 4° C. For sequential staining of β-gal and CK19, slides were stained as described for β-gal, mounted in PBS and imaged. Slides were then post-fixed in 4% PFA and incubated with anti-CK19 as described above with second image capture performed of the same area. β-gal⁺, CK19⁺, and HNF-4α⁺ cells were quantified by counting positive cells in ten 20× microscope fields and calculating the percent β-gal⁺/CK19⁺ and β-gal⁺/HNF-4α cells. The percent β-gal⁺/CK19⁻/HNF-4α⁻ cells was calculated as a percentage of the sum of all CK19⁺, HNF-4α⁺ and β-gal single⁺ cells. Two or three slides were examined for each BDL and DDC time point.

For co-localization of β-gal/desmin and β-gal/α-SMA, frozen sections were warmed to room temperature for 10 minutes, stained overnight in X-gal substrate solution and post-fixed with 4% PFA at room temperature for 10 minutes. Sections were rinsed in PBS and blocked with PBS/1% BSA at room temperature for 30 minutes. Sections were incubated with primary antibody at the following dilutions overnight at 4° C. (a-SMA 1:1600, Sigma, St. Louis, Mo., Clone 1A4, Cat #A2457; desmin 1:1000, Sigma, Clone DE-U-10 Cat #D1033). Sections were rinsed in PBS and incubated with Cy5-conjugated donkey anti-mouse IgG (Jackson ImmunoResearch Laboratories, Westgrove, Pa., Cat #715-175-500, 1:600 dilution). Sections were washed twice in PBS for 5 minutes, ddH₂0 twice for 5 minutes and mounted with fluorescent mounting media (KPL, Cat #71-00-16). Co-localization of β-gal/elastin was performed as above with the following modifications: After post-fixation with 4% PFA for 10 minutes at room temperature, sections were washed with TBS three times for 5 minutes followed by antigen retrieval with 0.5% hyaluronidase for 1 hour at room temperature and three 5 minute washes in TBS. Blocking was performed with TBS/3% BSA/0.9% NaCl in TBS at room temperature for 30 minutes. Sections were incubated with anti-elastin antibody (Cedarlane Laboratories Limited, Hornby, Ont, Cat #CL55041AP, 1:200 dilution) in TBS/3% BSA/1% normal goat serum, rinsed in TBS followed by a 30 minute room temperature incubation with Cy5-conjugated donkey anti-rabbit IgG 1:600 dilution (Jackson ImmunoResearch Laboratories, Cat #715-175-500), and mounted with fluorescent mounting media after rinses in PBS and water. Immunofluorescent and β-gal images were merged using digital “pseudocolorization” using the IP Lab imaging software application (BD Biosciences, Rockville, Md.).

### Laser Capture Microdissection, RNA Isolation and QPCR:

Harvested livers were washed in PBS, embedded in OCT, and frozen in a dry ice/ethanol bath. Four 8 μm serial sections were obtained using a Microm HM 505E (Richard Allen Scientific, Kalamazoo, Mich.) cryostat and placed on RNase free, membrane mounted metal-framed slides (Molecular Machines & Industries, Haslett, Mich.). Sections were brought to room temperature, placed in 75% EtOH for 30 sec, DEPC-treated water for 30 sec, Hematoxylin for 10 sec, DEPC-treated water for 15 sec, DEPC-treated water for 20 sec, twice in 95% EtOH for 30 secs, 100% EtOH for 30 sec, and xylene for 2 min and air-dried for ten seconds. An uncharged glass slide was placed over the polymer film and immediately processed for microdissection. Liver portal tracts and parenchymal tissues were collected using the SLμCut instrument (Molecular Machines & Industries) fitted to a Nikon Eclipse TE2000-S inverted microscope and collected on caps with adhesive lids (Molecular Machines & Industries, Haslett, Mich.). RNA was extracted using the PicoPure RNA Isolation Kit (Molecular Devices, Sunnyvale, Calif.) and samples (30 ng each) reverse transcribed using the WT-Ovation RNA Amplification System (NuGEN, San Carlos, Calif.). Quantitative PCR reactions were performed using SyBr Green QPCR Master Mix (Invitrogen, Carlsbad, Calif.) as described above.

### Statistical Analysis:

Students t-tests with equal variance and two-tailed distribution were used to determine the significance of differences between two groups (Excel statistical analysis software, Microsoft, Redmond, Va.). A p value of 0.05 was considered statistically significant. Results where indicated are expressed as mean±SE.

### Results

### Foxl1 is Activated in the Liver Following Injury

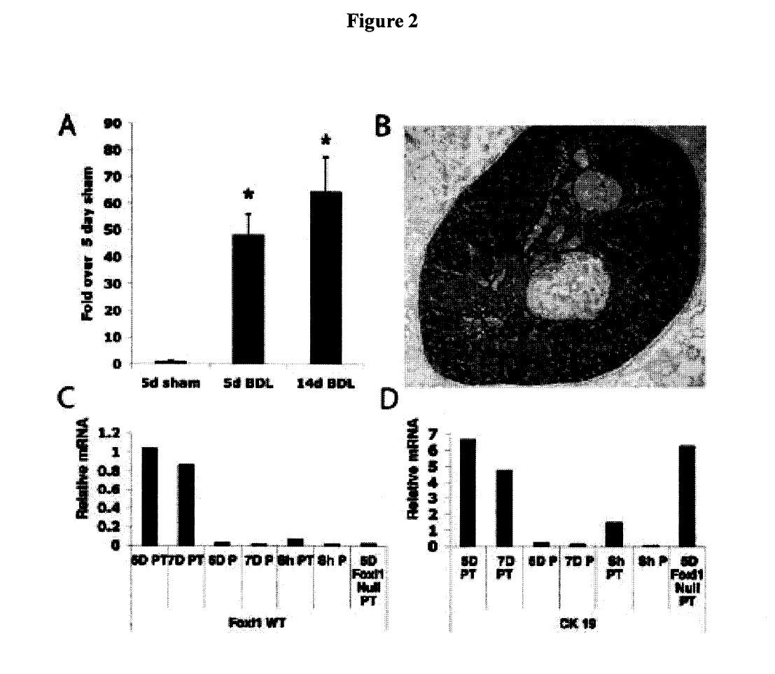

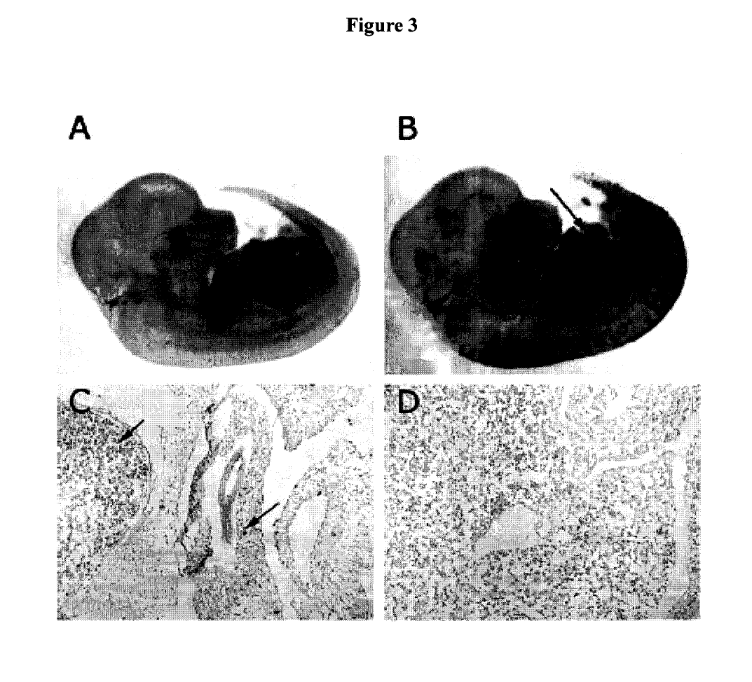

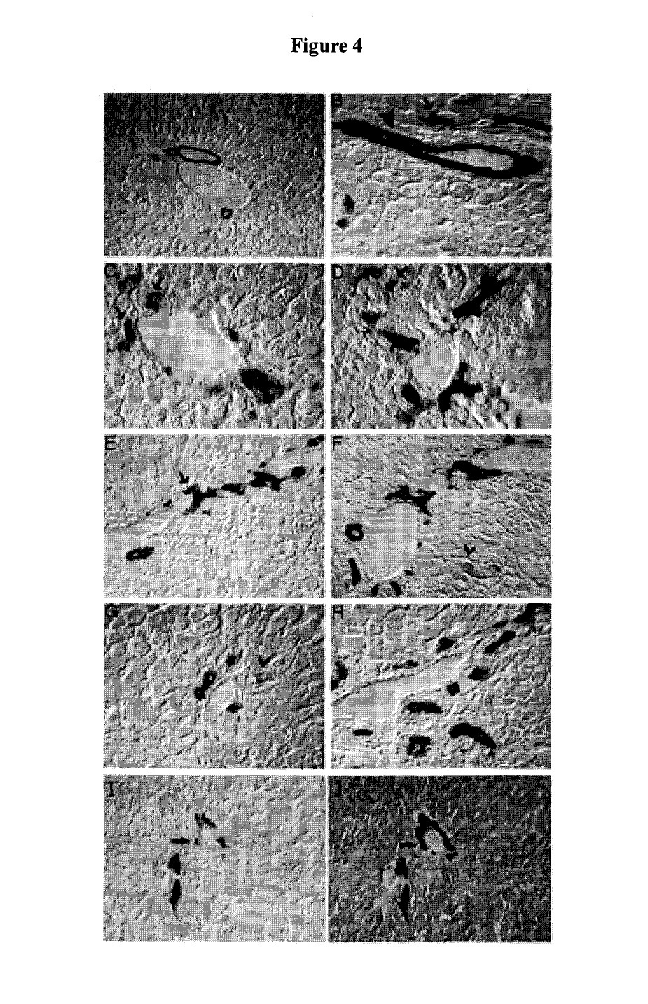

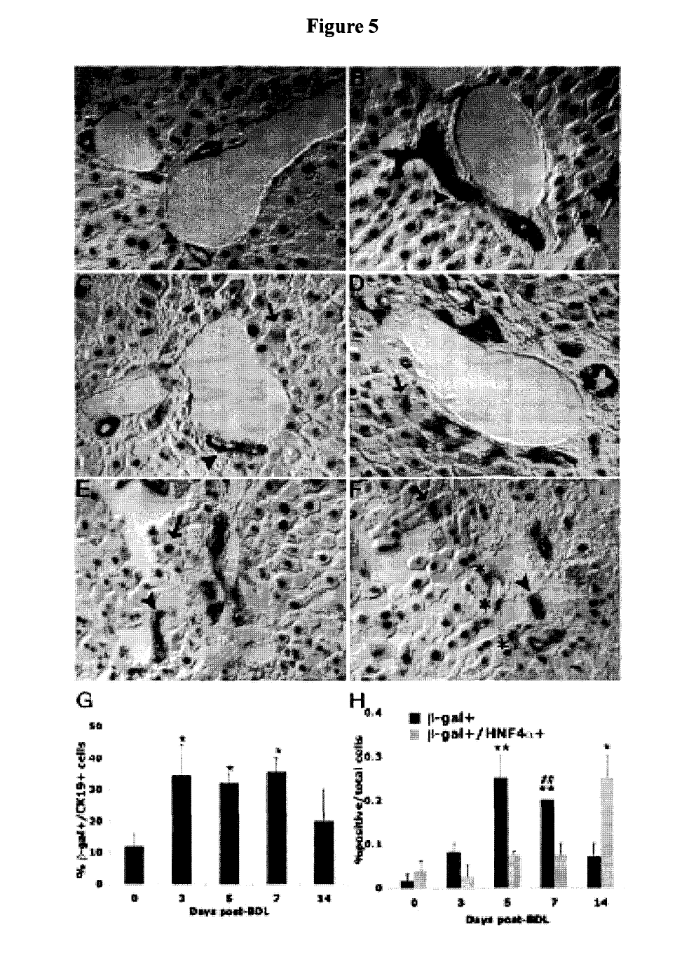

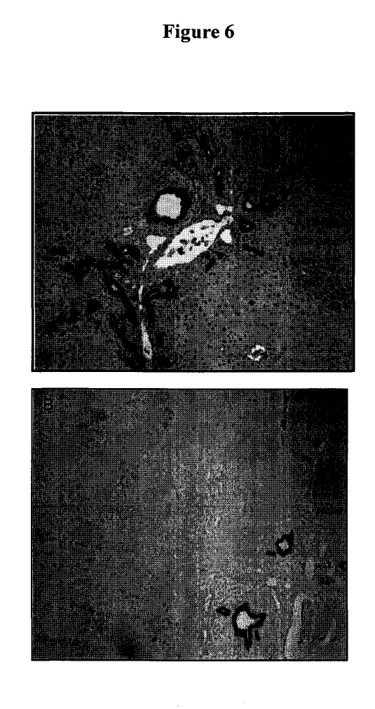

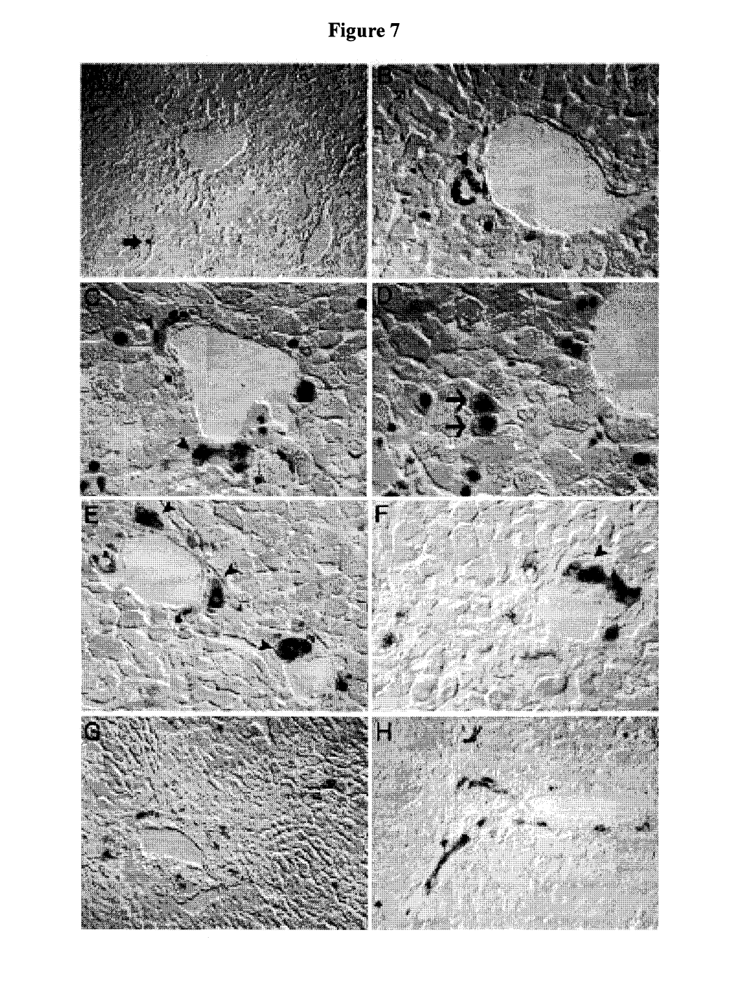

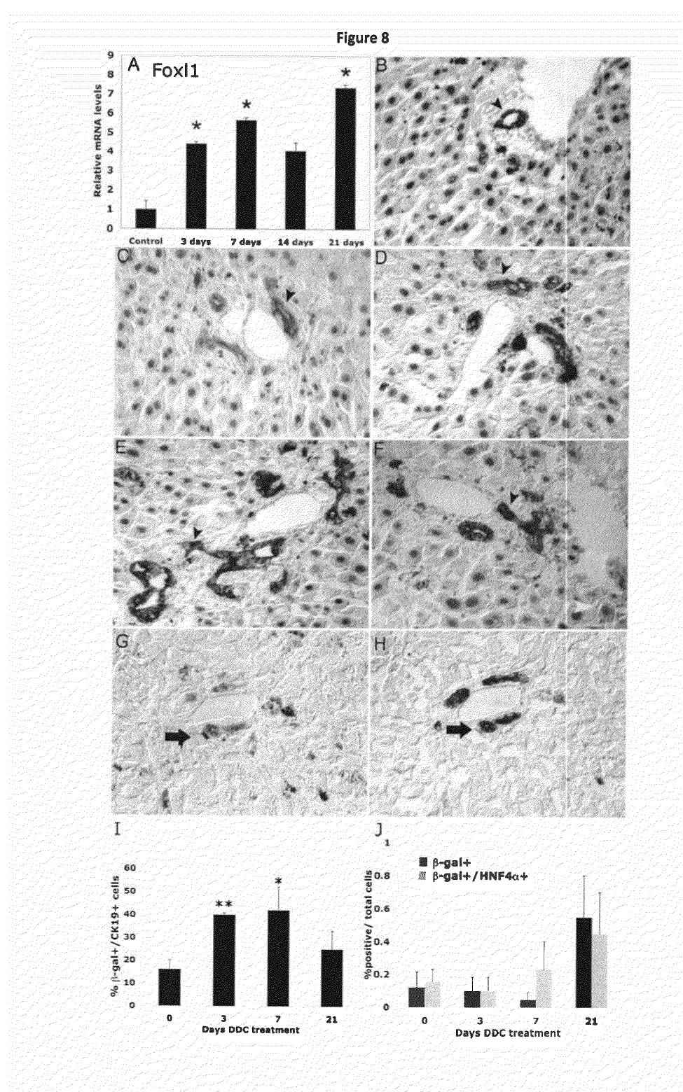

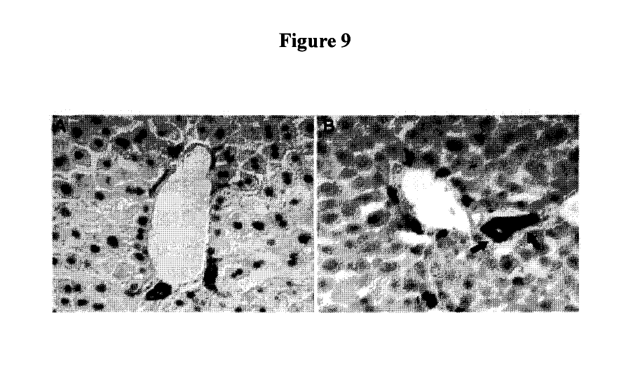

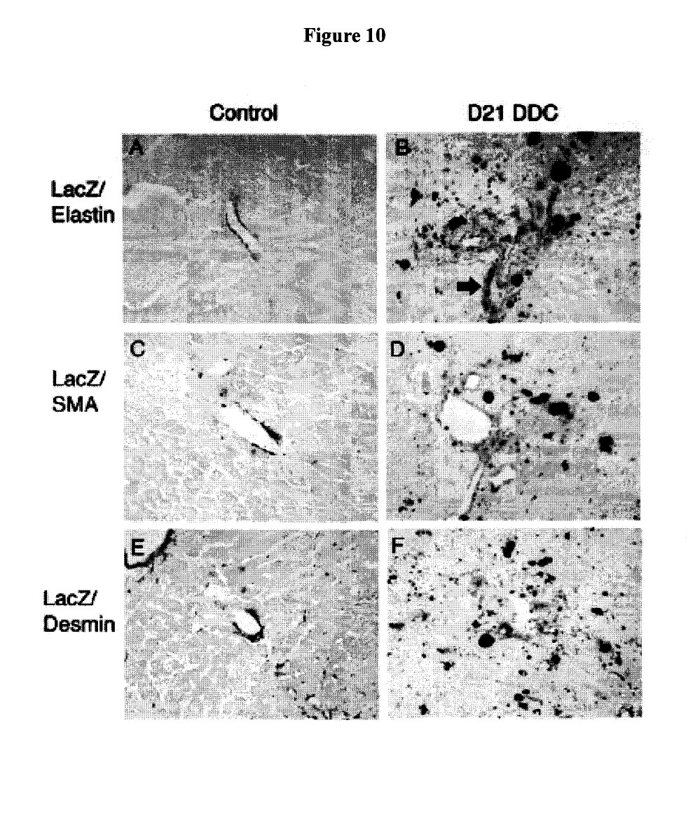

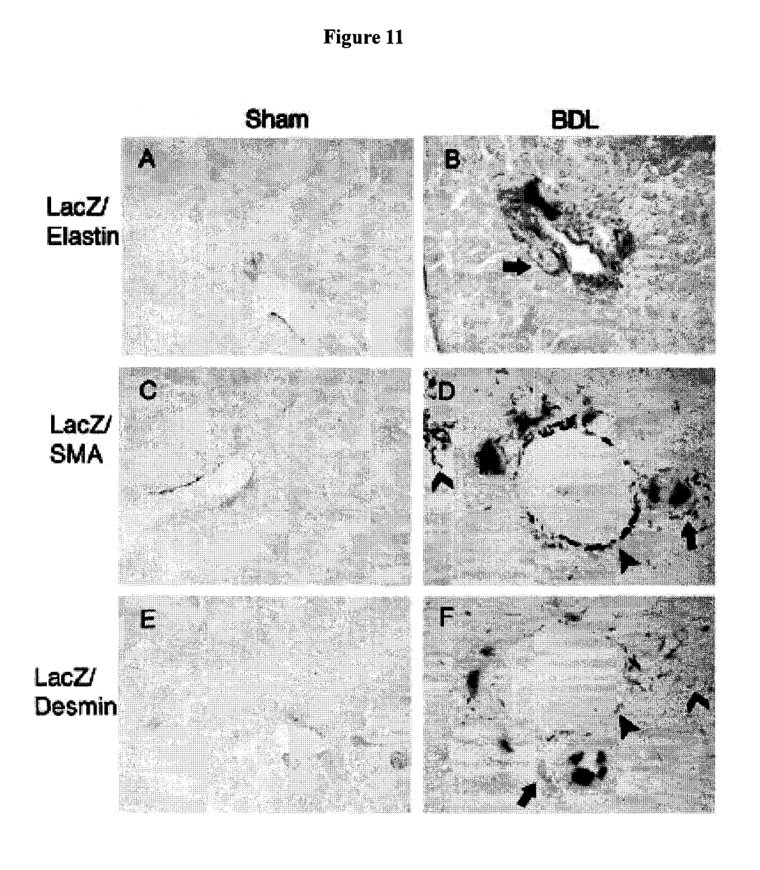

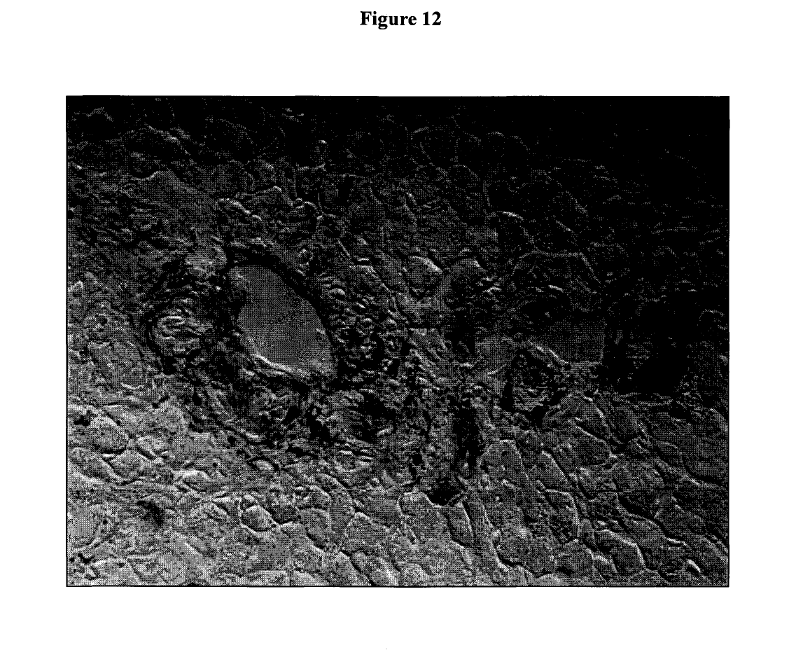

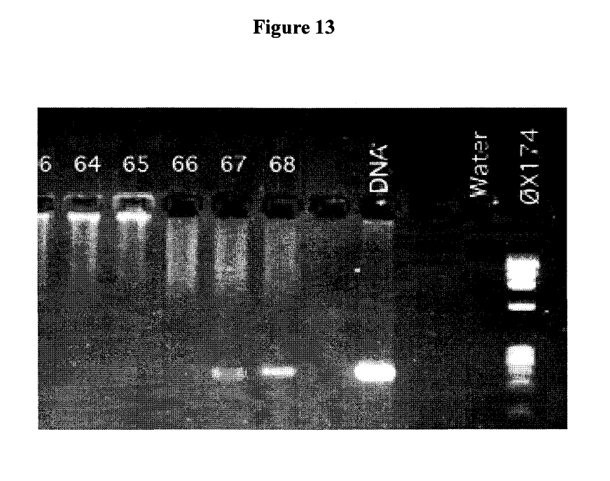

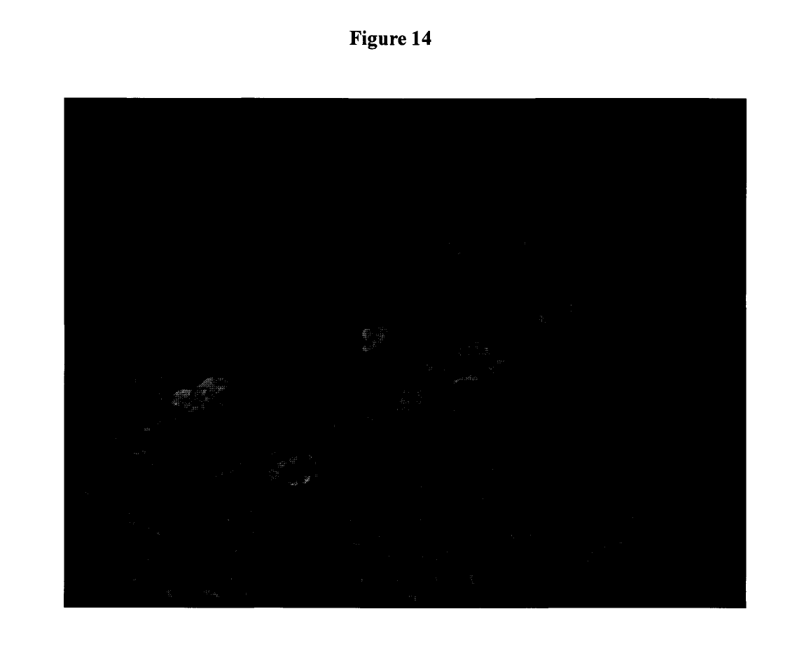

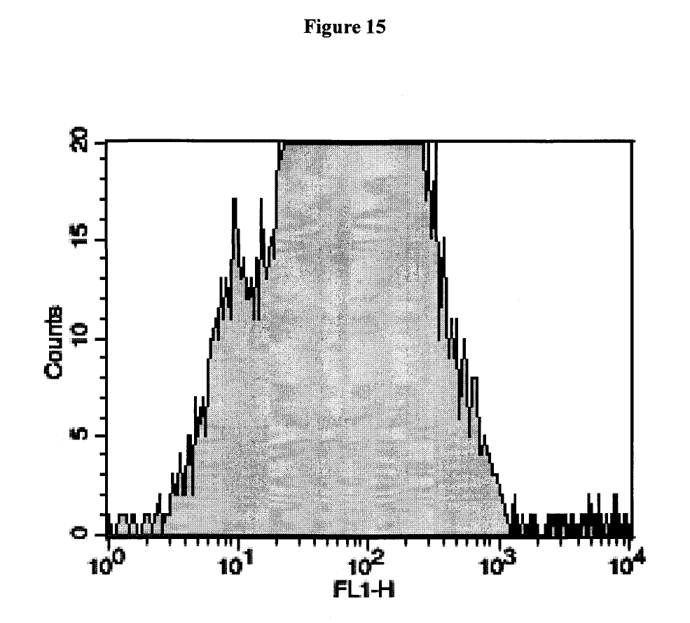

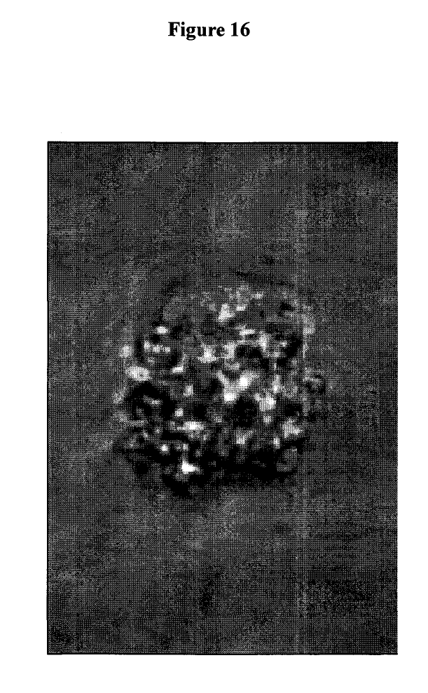

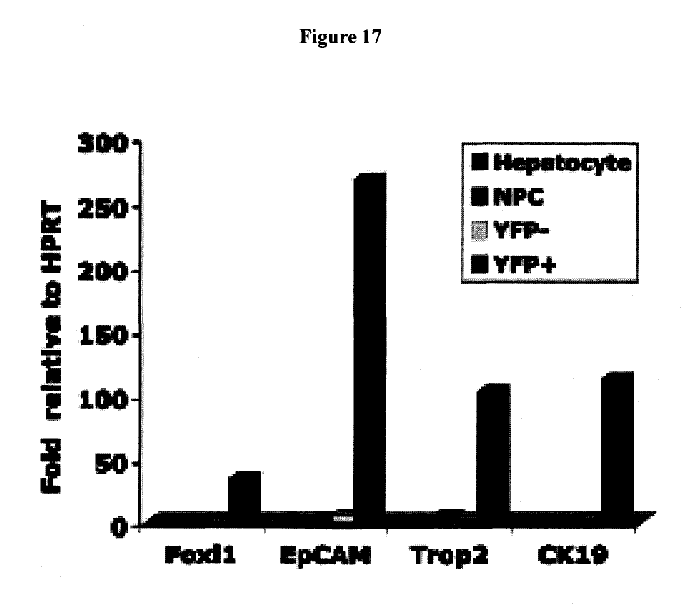

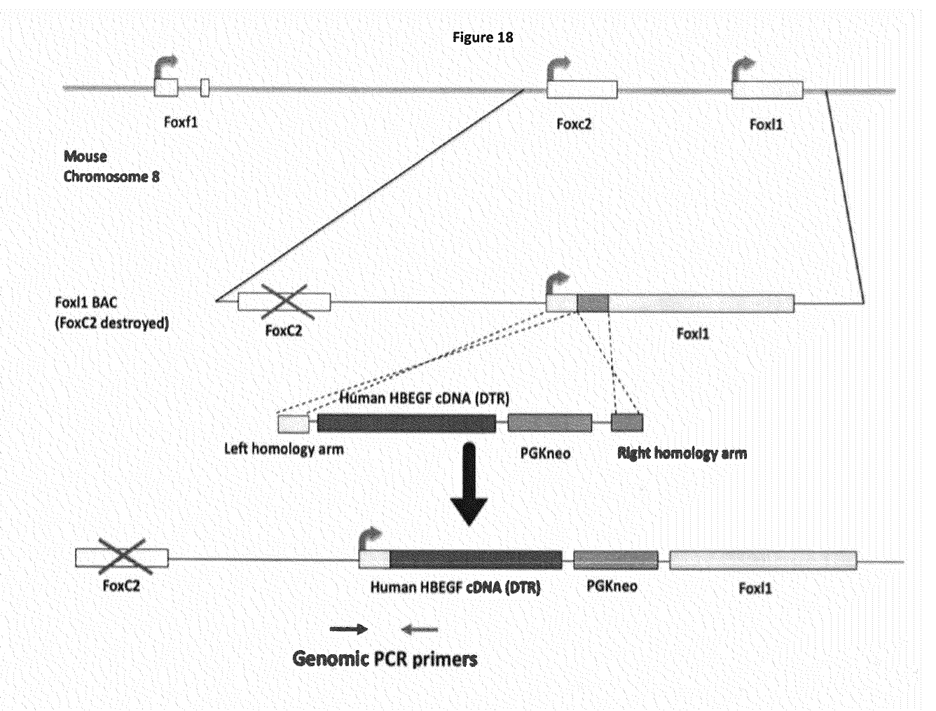

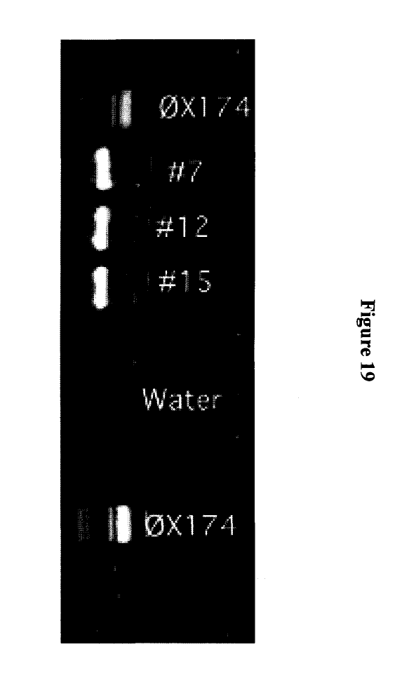

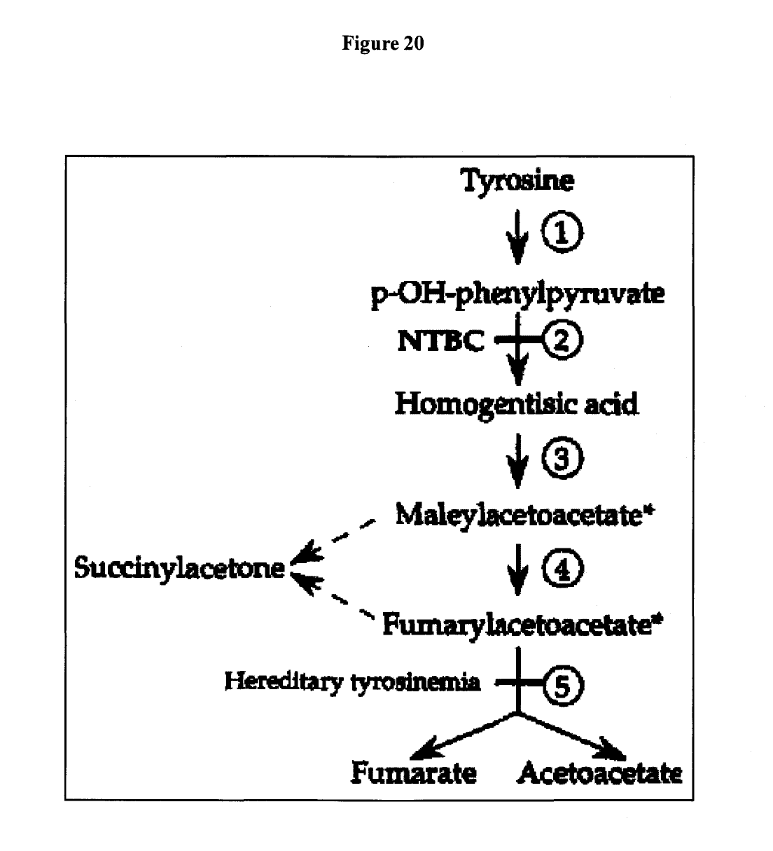

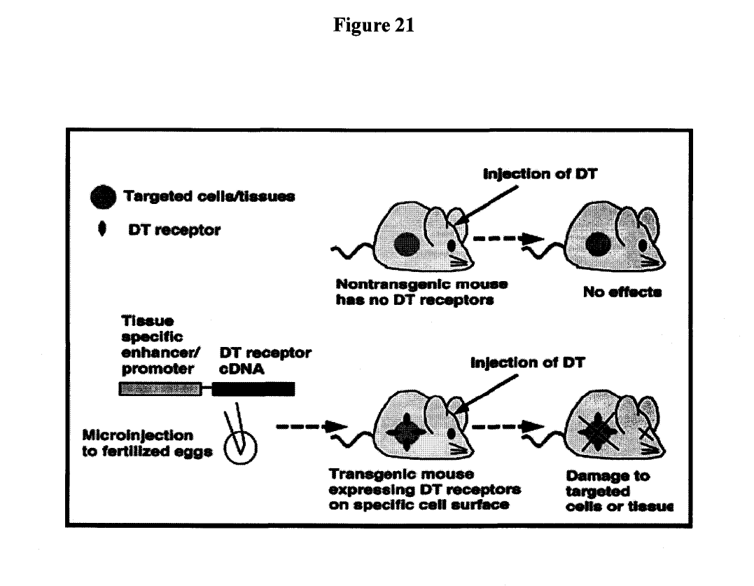

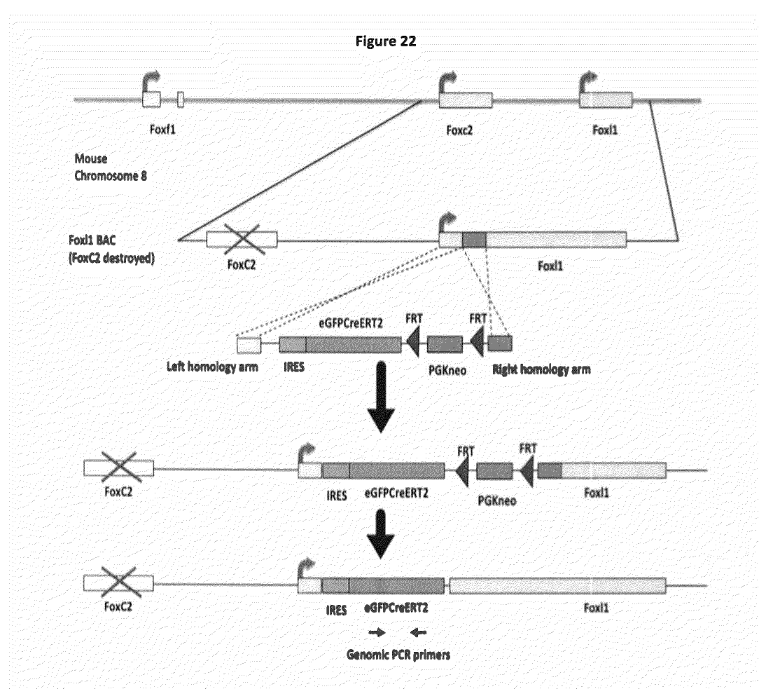

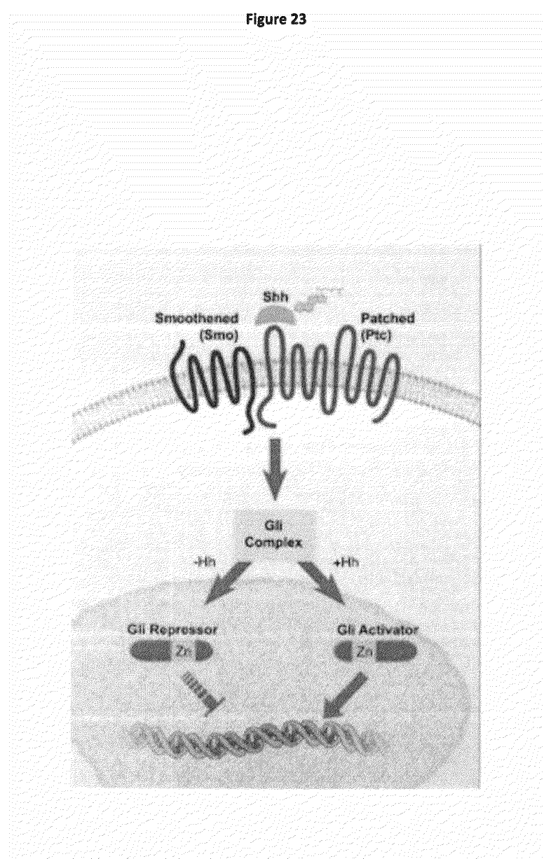

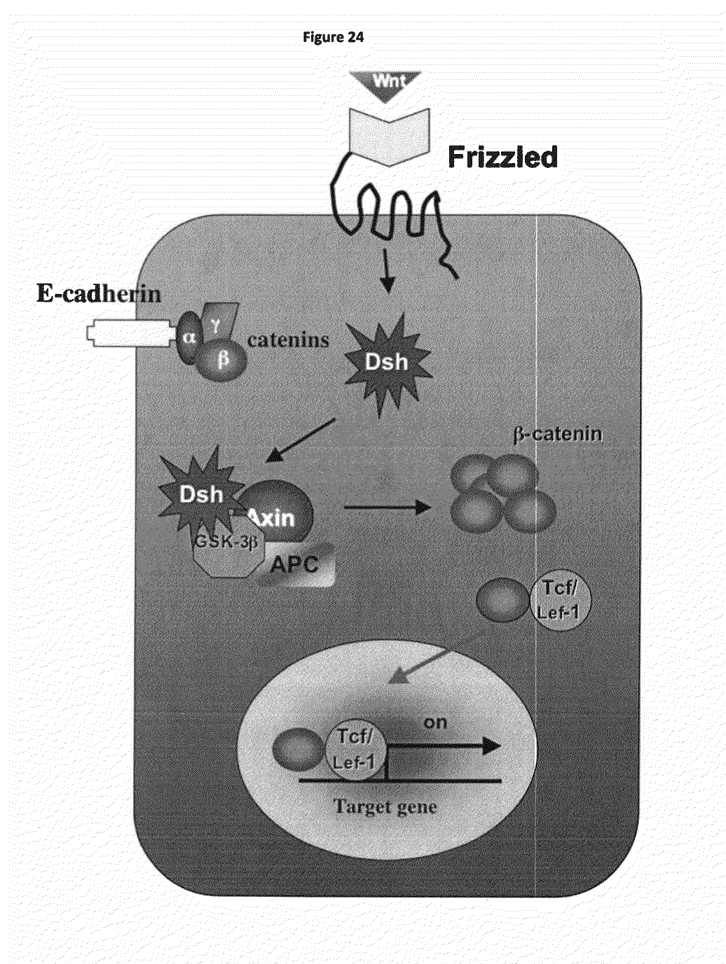

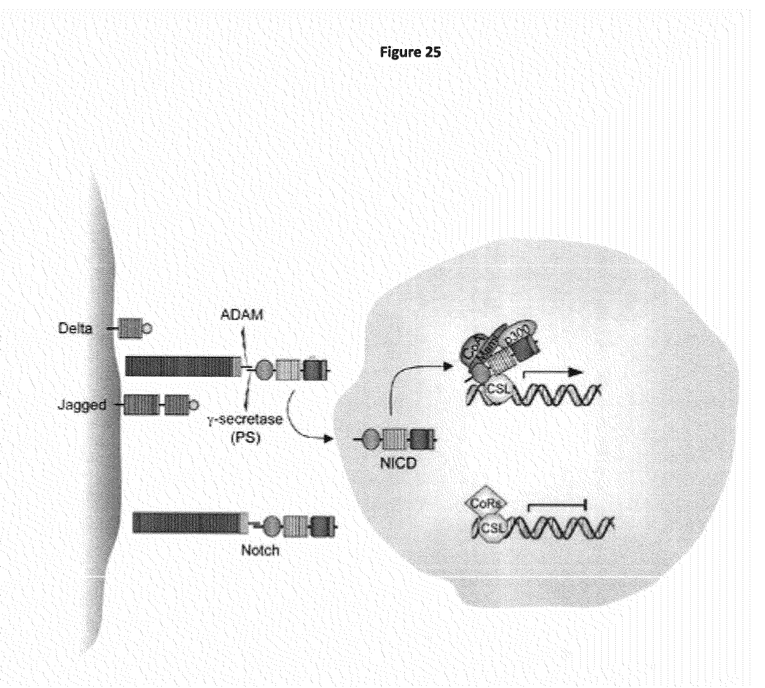

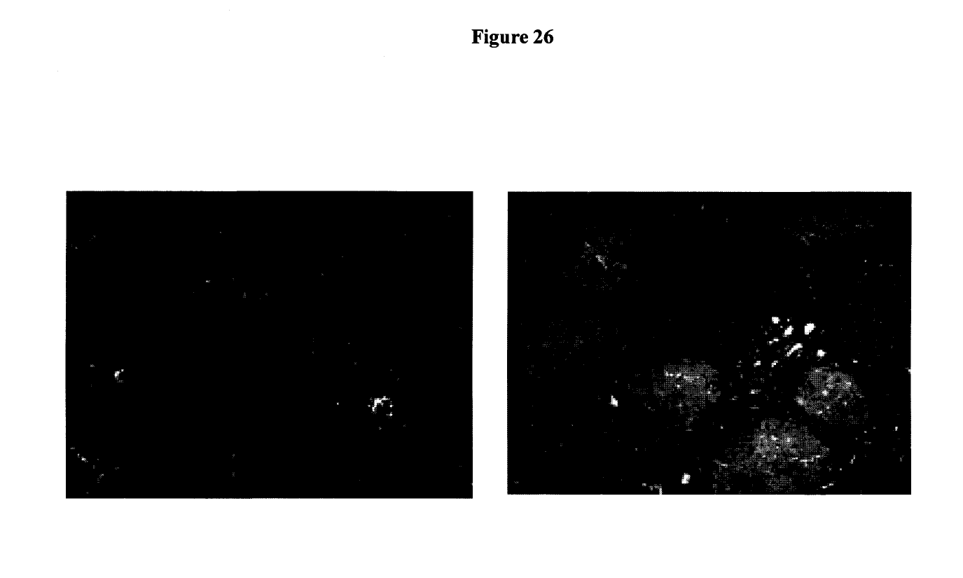

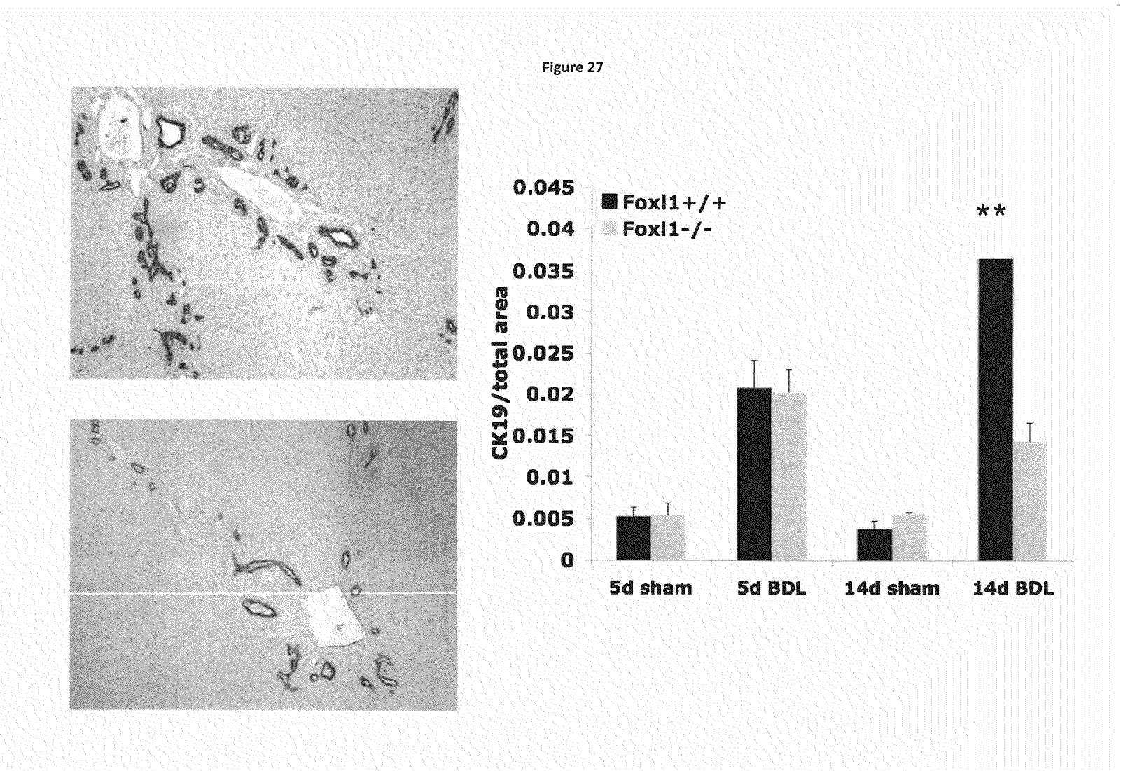

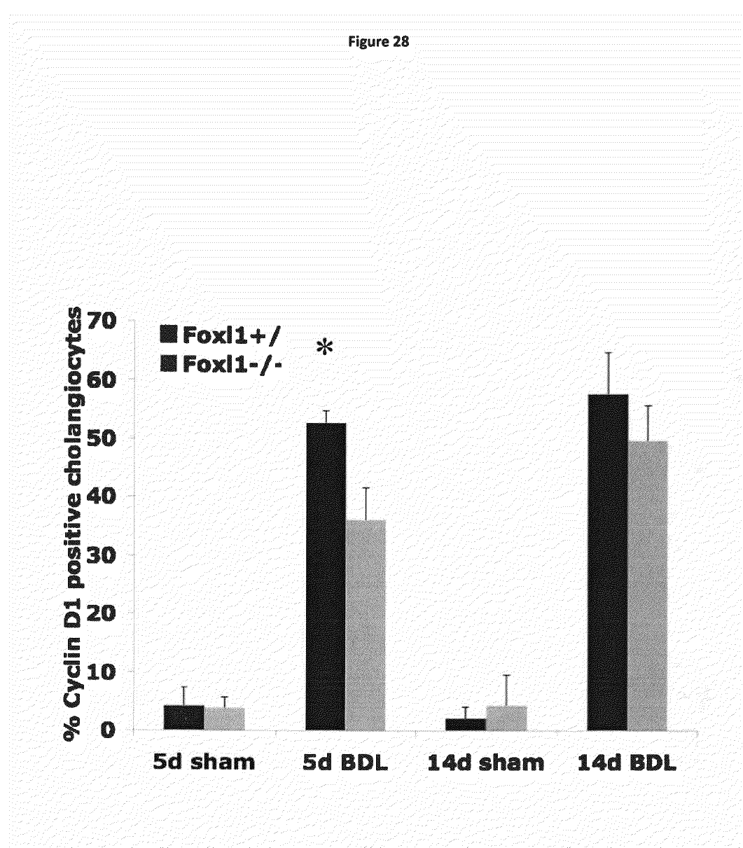

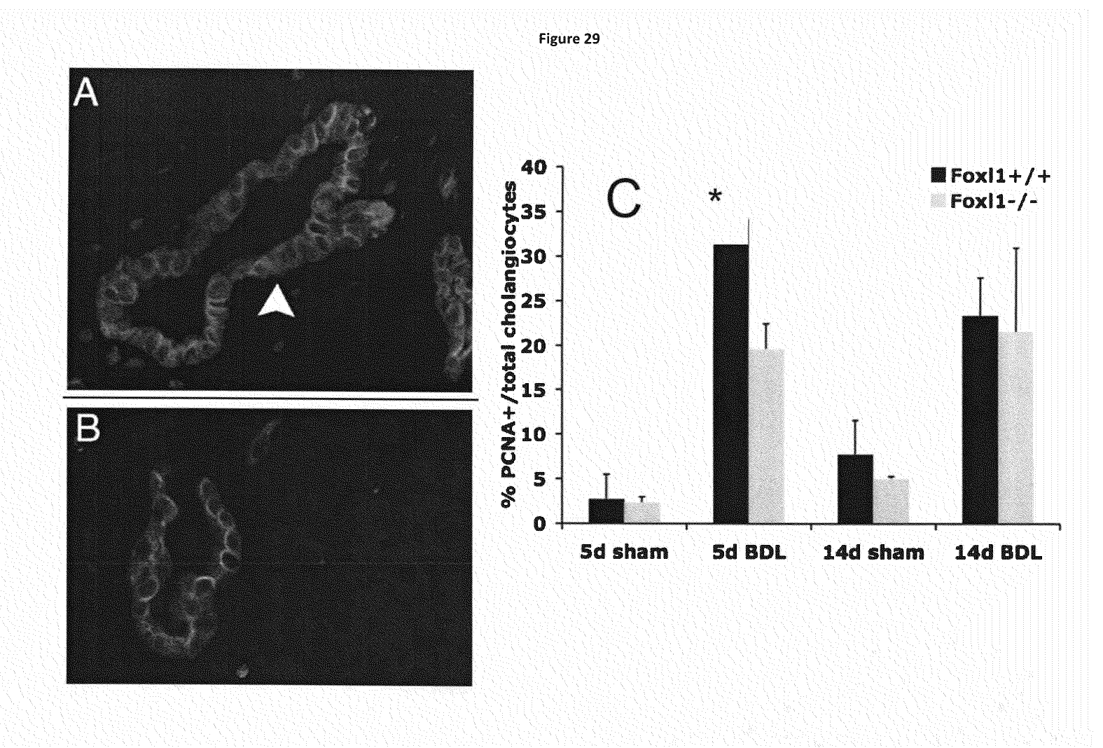

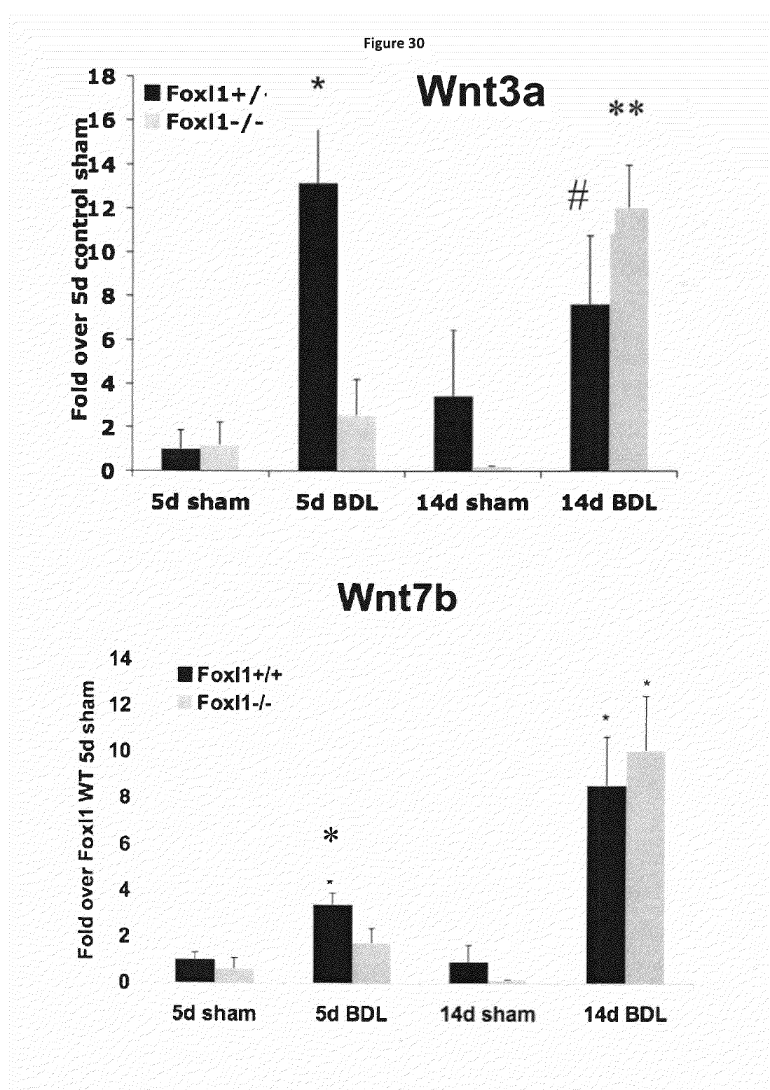

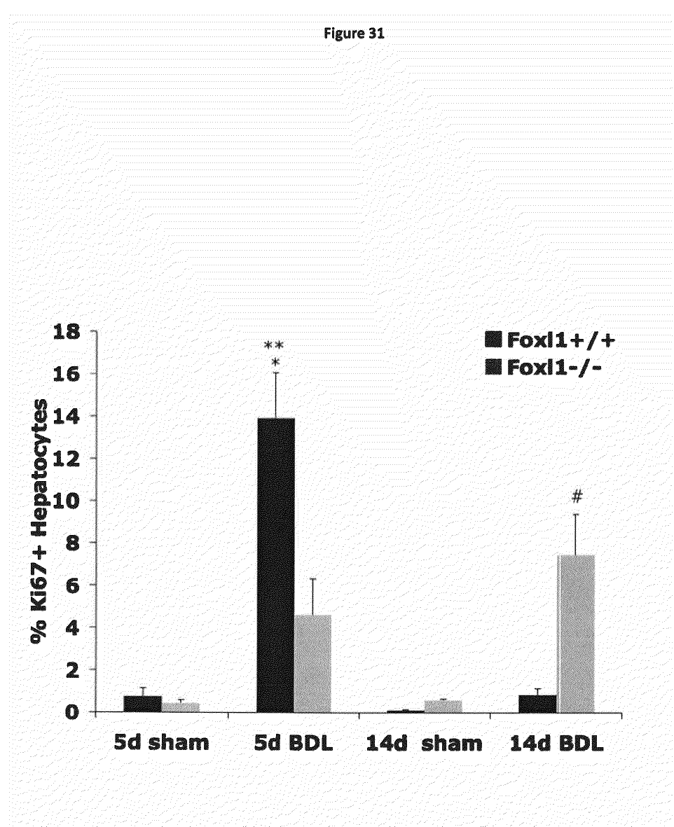

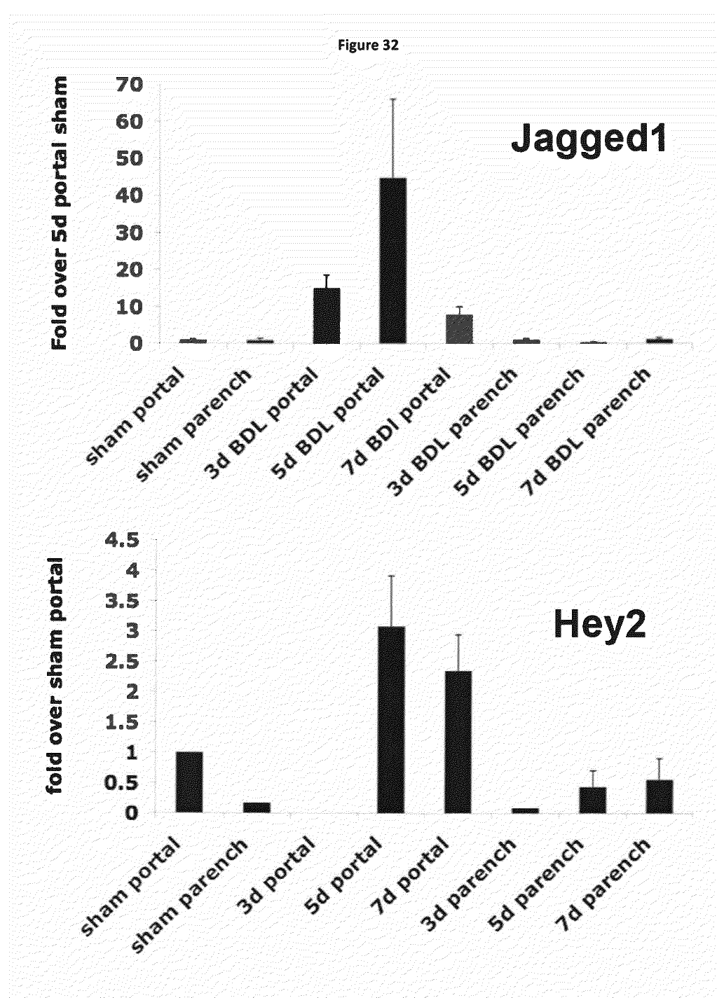

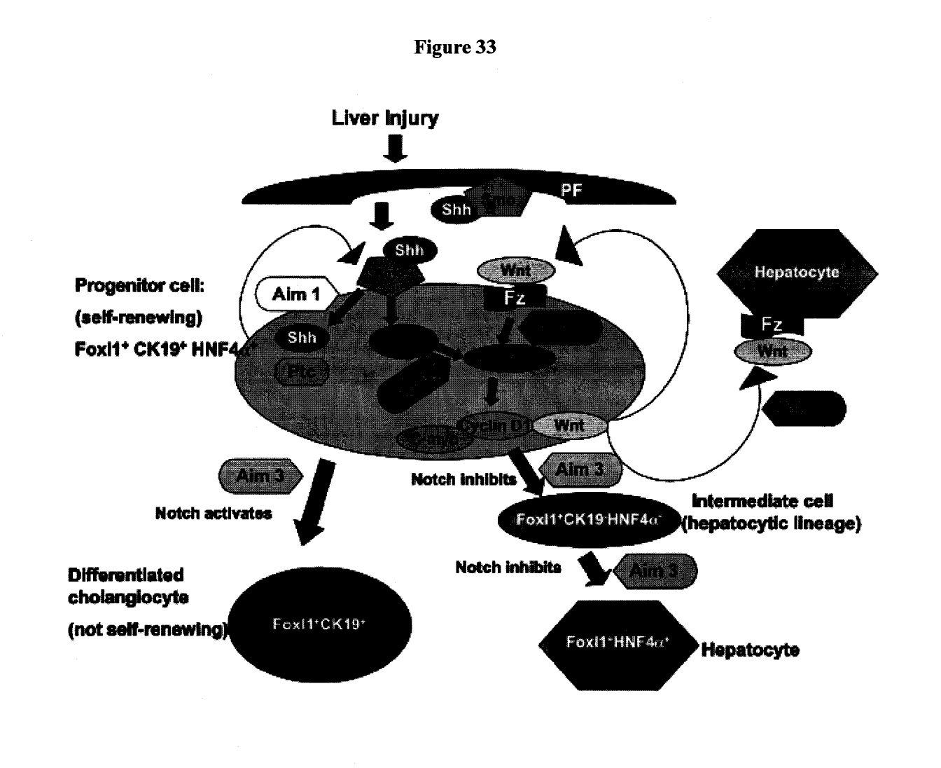

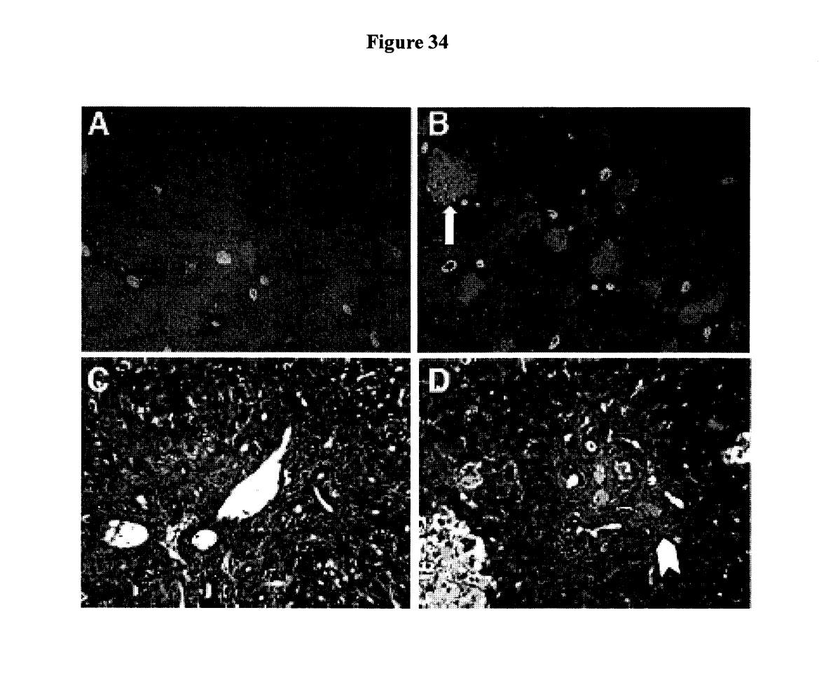

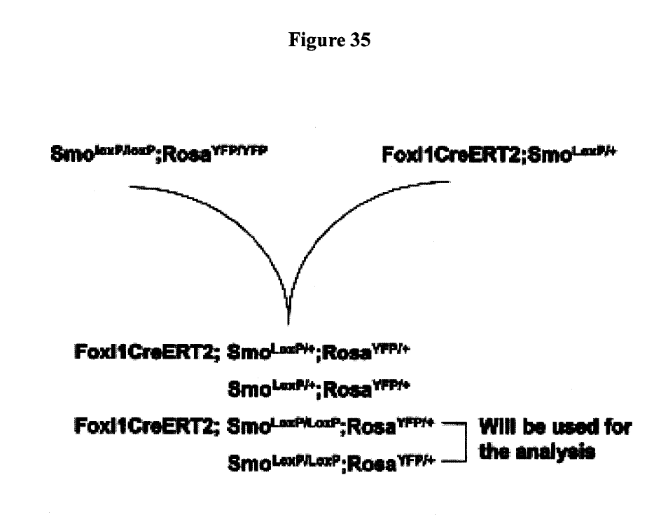

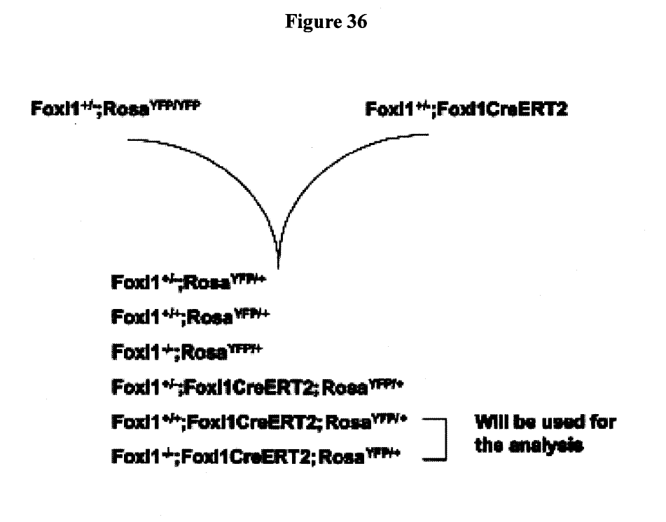

The mammalian forkhead box transcription factor, Foxl1 (GenBank Accession No: NM_{—}008024 for mouse; No: NM_{—}005250 for human; FIG. 1), is expressed in the gastrointestinal mesenchyme, but unlike its relatives of the Foxa and Foxo classes, its mRNA is not detected in the quiescent liver (Kaestner K H, et al, supra). Because Foxl1 is a marker of the mesenchyme in the intestine, and hepatic progenitor cells have been reported to express mesenchymal markers, Foxl1 mRNA expression was examined in the bile duct ligation (BDL) model by qRT-PCR. By way of background, in the BDL model, cholestatic injury induces a ductular reaction. Expression of Foxl1 was dramatically increased as compared to quiescent liver (FIG. 2A). To determine whether the expression of Foxl1 was confined to the portal tract region where the ductular reaction occurs, laser capture microdissection (LCM) was used to isolate portal tracts and adjacent parenchyma for gene expression analysis. Foxl1 expression was enriched in the portal tracts and absent from the surrounding parenchyma (FIG. 2B, C). As expected, Foxl1 expression was absent from portal tracts isolated from sham-operated animals or Foxl1 null mice (FIG. 2C). All samples were analyzed for cytokeratin 19 (CK19) expression to confirm minimal contamination of the parenchymal samples (FIG. 2D). These data show that the Foxl1 gene is activated in cells within or near the portal triad following cholestatic liver injury.

### Lineage Tracing of Foxl1-Positive Cells Following Bile Duct Ligation

In order to precisely localize Foxl1 expressing cells and their descendants in the injured liver, genetic lineage tracing with the Cre/loxP technology was employed. A BAC transgenic line in which expression of Cre recombinase is under the control of 170 kB of cis-regulatory elements of the murine Foxl1 gene recapitulates the expression pattern of Foxl1 in the gastrointestinal tract (Kaestner K H, et al., (2000) Genes Dev 14:142-146). The Foxl1-Cre line was crossed with Rosa26-lacZ reporter mice, in which beta-galactosidase (β-gal) is expressed only in those cells in which Cre has been active. The ontogeny of facultative bipotential progenitors in the liver was unknown. One possibility was that these cells are remnants of the fetal hepatoblast lineage, which differentiates into both hepatocytes and cholangiocytes in mid- to late gestation. Therefore, investigation into whether the Foxl1-Cre transgene is expressed in fetal hepatoblasts was pursued by collecting Foxl1-Cre; Rosa26R embryos on day 12.5 of gestation and performing β-gal staining. At this time point, the hepatic primordium is clearly visible but has not yet differentiated into mature cell types. As shown in FIG. 3, hepatoblasts were negative for β-gal, and thus for Foxl1-Cre expression, indicating that the Foxl1-positive progenitor in the adult liver is either not a remnant of the fetal hepatoblast population, or that this gene is activated in this lineage later in life.

In adult mice, a limited number of cholangiocytes were β-gal positive in the livers of sham-operated animals, consistent with the mRNA data shown in FIG. 2A (FIG. 4A). However, following BDL, β-gal activity was strongly induced, appearing first in isolated cells within ductular reactions adjacent to the portal vein (FIG. 4B), and increasing and extending away from the portal triad over time (FIG. 4C-H). Next, sequential staining and image capture was performed to confirm the colocalization of β-gal and CK19 (FIG. 4I and J). This expression pattern is consistent with that of a facultative hepatic progenitor cell that gives rise to both hepatocytes and cholangiocytes.

In order to confirm that Foxl1 marks bipotential progenitors, triple label experiments were performed with the livers of bile duct ligated Foxl1-Cre;Rosa26R mice for β-gal, CK19 and HNF-4α (hepatocyte nuclear factor 4 alpha), an orphan nuclear receptor specific for hepatocytes. The Foxl1-Cre positive lineage gives rise to both hepatocytes and cholangiocytes, consistent with the proposed role of Foxl1 as a marker of hepatic progenitor cells (FIG. 5). Quantitative analysis was performed on β-gal⁺ cells that coexpressed either CK19⁺, HNF-4α⁺ or were positive only for β-gal. As shown in FIG. 5G, the percentage of β-gal positive cells in bile ducts that co-stained with CK19 increased three-fold beginning 3 days after BDL surgery, consistent with an enrichment for Foxl1-Cre expressing cholangiocytes in this model. Five and seven days post-BDL, cells that were β-gal⁺/CK19^{-/}HNF-4α⁻ increased sixteen and thirteen-fold over sham levels, respectively (FIG. 5D, H). The number of cells that were β-gal⁺/HNF-4α+ increased six-fold 14 days after BDL, following the peak of β-gal⁺/CK19^{-/}HNF-4α⁻ cells (FIG. 5F, H). These data indicate that Foxl1-Cre lineage positive cells gave rise to mature hepatocytes through an intermediate cell stage in which neither CK19 nor HNF-4α are expressed (FIG. 5D). Cells co-staining for CK19 and either HNF-4α, or C/EBPα, another established marker of hepatoblasts/hepatocytes, were not observed (Yamasaki H, et al., (2006) Development 133:4233-4243). While the inability to detect triple positive cells does not exclude the possibility that they are present in low numbers in the Foxl1-Cre;Rosa26R mice, the data indicates that the cholangiocyte and hepatocyte fates are mutually exclusive in this model.

### Foxl1 is Required for the Proliferative Response of the Liver Following Injury

While the lineage tracing described above demonstrates that Foxl1 marks both the cholangiocytes and hepatocyte lineages after liver injury, it does not address the question whether Foxl1 expression is required for the proliferative response following liver injury. To address this question, bile duct ligation was performed on mice homozygous for a null allele of Foxl1 (Kaestner K H, et al., (1997) supra). Biliary proliferation was reduced and hepatic necrosis increased in Foxl1 mutant livers following BDL, as shown in FIG. 6, indicating the Foxl1 regulates essential targets in the regenerative response.

### The Foxl1-Positive Cell Lineage is Enriched for Proliferating Cells

Oval cells are proliferative cells that appear in the liver under conditions of injury when hepatocyte proliferation is limited. To determine if the Foxl1-Cre lineage is enriched for proliferating cells, dual-labeling studies for β-gal and the proliferation marker Ki-67 were performed. As expected, very few proliferating cells in the livers of sham-operated animals were detected (FIG. 7A). However, three days following BDL, cells singly positive for Ki-67 or β-gal in bile ducts and periportal areas were observed, and some double-positive cells in bile ducts (FIG. 7B). The frequency of double positive cells in the portal triad increased five and seven days following surgery; in addition, a few hepatocytes were found to be β-gal and Ki-67 positive in 5-day BDL animals (FIG. 7C, D). By 14 days post-surgery, there was an overall decrease in the number of proliferating cells (FIG. 7E-H). These data show that the Foxl1-Cre marked lineage proliferates following BDL, consistent with a facultative progenitor phenotype.

Foxl1l-Cre Activation Following DDC Diet

In order to demonstrate that activation of the Foxl1 locus is not unique to bile duct ligated livers, the DDC model of liver injury, which has been shown to induce a massive oval cell response in rodents, was also tested (Preisegger K H, et al., (1997) Lab Invest 79:103-109; Swenson E S, et al., (2008) Liver Int 28:308-318; Petersen B E, et al., (2003) Hepatology 37:632-640). As demonstrated in FIG. 8A, Foxl1 mRNA levels were significantly increased in mice fed a DDC diet, although the fold-change was less than that seen in the BDL model. Furthermore, β-gal-positive cells were significantly increased in the DDC injured liver, again within ductular reactions (FIG. 8B-H). Additionally, subsets of the β-gal positive cells also expressed CK19 or HNF-4α (FIG. 8F), consistent with the notion that the Foxl1-Cre transgene marks bipotential progenitors in the injured liver. Similar to the BDL model, a 2.6 fold increase in the percentage of β-gal positive cells in bile ducts that co-stained with cytokeratin 19 after seven days of DDC-induced injury was detected (FIG. 81). However, in contrast to the BDL model, the number of single β-gal positive and β-gal, HNF-4α double positive cells did not increase significantly during DDC injury (FIG. 8J), which may reflect the inter-animal variability in response to this dietary genotoxin. β-gal and CK19 were also coexpressed in a subset of CK19 cells in mice that received a choline deficient, ethionine supplemented (CDE) diet, another well established model associated with hepatic progenitor cell proliferation (FIG. 9). Thus, the Foxl1 gene is activated in three models of liver injury, and marks cells that ultimately express genes specific to the mature epithelial cells of the liver.

### Foxl1-Expressing Cells are Closely Apposed to Portal Fibroblasts

Foxl1 expression in the intestinal tract is restricted to the mesenchyme (Kaestner K H, et al., (1997) supra; Perreault N, et al., (2001) J Biol Chem 276:43328-43333; Perreault N, et al., (2005) Genes Dev 19:311-315). Further, portal fibroblasts and hepatic stellate cells are located in close proximity to progenitor cells and can signal in a paracrine fashion to hepatic progenitor cells (Ramadori G, et al., (1992) Biochem Biophys Res Commun 183:739-742; Cassiman D, et al., (2002) J Hepatol 36:200-209; Cassiman D, et al., (2001) Hepatology 33:148-158). Therefore, the localization of Foxl1 expression in relationship to portal fibroblasts and hepatic stellate cells in two injury models was investigated. Significant expression of elastin, a specific marker of portal fibroblasts in DDC-injured livers was detected (FIG. 10B) (Li Z, et al., (2007) Hepatology 46:1246-1256; Lorena D, et al., (2004) Lab Invest 84:203-212). There was minimal expression of desmin (a marker of hepatic stellate cells) and α-SMA (a marker of both portal myofibroblasts and hepatic stellate cells which have undergone myofibroblastic differentiation) in the DDC injured livers, indicating that this paradigm does not stimulate significant activation of hepatic stellate cells or other myofibroblasts (9D, F). Notably, elastin positive cells were increased in number and encircled the β-gal positive cells, but did not co-stain the same cell population. In BDL injured livers (FIG. 11), significant expression of elastin, desmin and α-SMA was detected (FIG. 11, B, D, F). Similar to the DDC model, there was no overlap between Foxl1-Cre (β-gal) expression and the markers of either portal fibroblasts or hepatic stellate cells. The close proximity of portal fibroblasts to Foxl1-Cre positive progenitor cells in both DDC and BDL paradigms indicates that portal fibroblasts rather than hepatic stellate cells participate in paracrine signaling with Foxl1-Cre positive progenitors.

### Discussion

Identification of unique oval cell markers that can be used to isolate resident hepatic progenitors has been impeded by their heterogeneity and by the fact that the majority of markers identified to date are shared by other cell types within the liver (Jelnes P, et al., (2007) Hepatology 45:1462-1470.). Foxl1 is expressed in a limited number of cells in the normal adult liver, but is dramatically induced in two established models of liver injury associated with hepatic progenitor cell proliferation. Thus, Foxl1 is a bona fide marker of the facultative progenitor cells.

Progenitor cells proliferate in close proximity to hepatic stellate cells and portal fibroblasts in chronic liver injury, indicating that they constitute a progenitor cell niche that influences differentiation and proliferation (Cassiman D, et al., (2002) J Hepatol 36:200-209; Cassiman D, et al., (2001) Hepatology 33:148-158). In contrast to its intestinal expression in the mesenchyme, Foxl1 is not expressed in either hepatic stellate cells or portal fibroblasts. These results are consistent with those of Yovchev and colleagues whose characterization of oval cell lineages indicate that a subpopulation of hepatic progenitor cells expressing markers of a mixed epithelial-mesenchymal lineage were able to repopulate injured rat liver and were distinct from thy-1 positive portal fibroblasts and hepatic stellate cells (Yovchev M I, et al., (2008) Hepatology 47:636-647.).

A better understanding of the cellular organization of the hepatic stem cell niche is essential for elucidating hepatic progenitor cell behavior in response to liver injury. Kuwahara and colleagues have used label retention assays in combination with acetaminophen-induced liver injury to identify four predicted sources of hepatic progenitors (Kuwahara R, et al., (2008) Hepatology 47:1994-2002). Although different liver injury paradigms were employed herein, the locations of Foxl1-Cre positive cells in the BDL, DDC and CDE livers were similar to a subset of the label-retaining cell populations identified in Kuwahara's analysis, specifically, the peribiliary hepatocytes and intraductular cells. The quantitative analysis demonstrating an increase in the number of Foxl1-Cre⁺/CK19⁻/HNF-4α⁻ cells preceding the increase in Foxl1-Cre⁺/HNF-4α⁺ positive cells is consistent with a model in which Foxl1-Cre progenitor cells progress through an intermediate stage in their differentiation to mature cholangiocytes and hepatocytes.

The co-staining studies indicate that portal fibroblasts are located in close proximity to Foxl1-Cre progenitor cells in both injury paradigms investigated, and in many instances these cells appear to encircle the Foxl1-Cre positive cells. The juxtaposition of Foxl1-Cre-expressing progenitor cells and portal fibroblasts indicates the potential for paracrine signaling between these two cell populations. At the present time, the signals that direct hepatic bipotential progenitor differentiation to either hepatocyte or cholangiocyte lineages are unknown. The potential for crosstalk between hepatic progenitor cells and portal fibroblasts has not been investigated in vivo, due in part to the lack of suitable genetic models. However, recent studies have demonstrated that hedgehog mediated paracrine interactions between hepatic stellate cells and hepatic progenitors are required for hepatic progenitor cell viability and proliferation (Jung Y, et al., (2008) Gastroenterology 134:1532-1543; Fleig S V, et al., (2007) Lab Invest 87:1227-1239). Preliminary studies indicate that Foxl1 is a downstream target of hedgehog signaling in the intestine. These findings indicate that Foxl1 may also be a critical mediator of hedgehog signaling cross talk with portal fibroblasts in the liver, and could be involved in the maintenance of liver progenitor cell viability, proliferative response to injury and/or differentiation. Foxl1^{-/-}; Foxl1-Cre;Rosa26R mice may be used for further investigation into the role of Foxl1 in hepatic progenitor lineage commitment by determining if the mice exhibit differences in cell lineage allocation during recovery from liver injury.

While hepatic progenitors possess some characteristics of both epithelial and mesenchymal lineages (Yovchev M I, et al., (2008) Hepatology 47:636-647; Inada M, et al., (2008) J Cell Sci 121:1002-1013.), the absence of co-localization of β-gal with myofibroblast markers argues strongly against the possibility that Foxl1-Cre progenitor cells have undergone a lineage switch to a myofibroblastic cell in either BDL or DDC injury paradigms within the time frame examined. Nevertheless, the activation of Foxl1, a mesenchymally expressed gene in the gastrointestinal tract, in hepatic progenitors could be important for the acquisition of mesenchymal characteristics associated with epithelial-mesenchymal transition (EMT), such as increased motility and invasiveness, cellular characteristics that facilitate migration of progenitors to sites of liver injury.

The preceding demonstrates that Foxl1 indeed marks facultative stem cells and their descendants in the liver and identified a distinct hepatic progenitor cell population which differs from both hepatic stellate cells and portal fibroblasts.

### Example 2

In light of the development of the Foxl1-Cre mouse, and the demonstration that Foxl1-Cre positive cells contribute to both the mature hepatocyte as well as cholangiocyte lineages during the liver's recovery from injury, it is possible to explore the potential of these bipotential progenitor cells for cell replacement therapy in the transplant setting, as well as to determine their contribution to the liver's response to injury. Furthermore, the novel genetic tools described herein will allow unequivocal determination whether hepatocellular carcinoma is derived from resident hepatic stem cells. In this regard, adult hepatic stem cells will be isolated and tested for their clonogenic and differentiation potential in vitro and in vivo, the contribution of the Foxl1-positive stem cell lineage to the proliferative response following liver injury will be assessed. Additionally, the role of the Foxl1-positive hepatic stem cell lineage in hepatocellular carcinoma will be evaluated.

By way of background, patients suffering from any of a number of genetic and acquired chronic disorders of the liver could benefit from cell replacement therapy, preferably with committed hepatic progenitor cells. In the healthy adult liver, turnover of the two major cell types, the biliary epithelial cell or cholangiocyte and the hepatocyte, is very slow. In fact, in the adult liver, only one out of every 20,000 hepatocytes is mitotic at a given time, and as a result, the liver replaces itself only every one to two years (Bucher, N. L. R., et al. 1971, Boston, Mass.: Little, Brown and Co; Steiner, J. W., et al., (1966) Exp Mol. Pathol. 5: 146-181). However, when the liver is injured, mature epithelial cells reenter the cell cycle and restore liver mass and homeostasis (Engelhardt, N. V., et al., (1984) Histochemistry 80: 401-407; Grisham, J. W. (1962) Cancer Research 22: 842-849; Higgins, G. M., et al. (1931) Archives of Pathology 12: 186-202; Stowell, R. E., et al., (1950) Arch Pathol 50: 519-537). Serial transplantation experiments have demonstrated that healthy hepatocytes are able to expand in clonal fashion when transplanted into a diseased liver (Overturf, K., et al., (1999) Nat Genet 12: 266-273). However, in patients or animal models with chronic liver disease the capacity of hepatocytes and cholangiocytes to proliferate is blocked and small bipotential progenitors are activated (Alison, M. R., et al., (1998) Cell Prolif 38: 407-421; Bisgaard H C, et al., (2002) Am J Pathol 161:1187-1198; Fausto N. (2004) Hepatology 39:1477-1487; Tan J, et al., (2002) Liver 22:365-373; Thorgeirsson S S. (1996) Faseb J 10:1249-1256). As alluded to, these facultative stem cells, described as “oval cells” in rodent models or hepatic progenitors in humans, are believed to represent the remnants of the ductal plate that develops into the canal of Hering in pediatric and adult livers. Oval cells cannot be readily detected in the quiescent liver but are frequently found in structures known as “ductular reactions” in both experimental models of liver injury and patients with massive acute hepatic necrosis as well as chronic liver disease. These ductular reactions can contain undifferentiated cells as well as intermediate cells of both cholangiocyte and hepatocyte lineages (Zhou H, et al., (2007) Hepatology 45:716-724). Some investigators believe that the ductal plate cells represent pluripotent stem cells that give rise to transit amplifying bipotential progenitors (oval cells) (Zhang L, et al., (2008) Hepatology 48:1598-1607). Thus, the origin of oval cells, their normal location within the liver and physiologic behavior are only partially known. Nevertheless, these progenitors hold the potential to be useful for the restoration of liver function in patients with chronic or even acute liver disease (Dorrell C, et al., (2008) Hepatology 48:1282-1291; Dorrell C, et al., (2005) Stem Cell Rev 1:61-64; Grompe M. (2005) Gastroenterology 128:2158-2160; Sell S. (2001) Hepatology 33:738-750; Sell S, et al., (2008) J Clin Oncol 26:2800-2805; Zaret K S, et al., (2008) Science 322:1490-1494).

The stem cell niche has been defined as the specialized microenvironment that “directly promotes the maintenance of stem cells.” (Morrison S J, et al., (2008) Cell 132:598-611). In the niche, direct contact between the stem cell and supporting cells in their immediate microenvironment is thought to be responsible for maintaining the balance between stem cell self-renewal and differentiation (Nusse R. (2008) Cell Res 18:523-527). Although the cell populations constituting the hepatic stem cell niche have not been identified with certainty, current models propose that hepatic stem and/or bipotential progenitor cells are supported by resident mesenchymal populations. The demonstration that Foxl1-expressing progenitor cells are within close proximity to portal fibroblasts in both BDL and DDC injury models (Sackett S D, et al., (2009) Hepatology 49:920-929) is consistent with the notion that portal fibroblasts participate in paracrine signaling with hepatic stem cells within the niche. Other cell types that are believed to be located within the hepatic stem cell niche include hepatic stellate cells, endothelial cells, hepatocytes, cholangiocytes, Kupffer cells, pit cells and inflammatory cells, with each of these cell types having the potential to signal to progenitor cells via paracrine signaling to influence progenitor cell behavior. For example, hepatic stellate cells are a source of growth factors including TGF-α, HGF and acidic FGF, proteins that have been implicated in crosstalk with hepatic progenitor cells during the course of liver injury (Kuwahara R, et al., (2008) Hepatology 47:1994-2002; Sanchez-Munoz D, et al., (2007) Dig Liver Dis 39:262-266; Santoni-Rugiu E, et al., (2005) Apmis 113:876-902.). There is a close correlation between the extent of hepatocellular injury, progenitor cell expansion and fibrosis. However, at present it is not known whether progenitor cell expansion drives fibrosis or whether these two processes occur independently.

A recent study raises the possibility that the deposition of extracellular matrix may actually precede and be necessary for progenitor cell expansion (Van Hul N K, et al., (2009) Hepatology 49:1625-1635.). Although intriguing, this study was descriptive and will require additional in vivo studies with modified genetic mouse strains in order to establish a causal relationship. Hepatic progenitor cells express markers in common with the cholangiocytic lineage raising the possibility that many of the properties associated with reactive cholangiocytes may also be part of the progenitor cell repertoire.

Among these characteristics are the regulation of cholangiocyte proliferation by growth factors, their ability to secrete autocrine and paracrine factors that activate hepatic stellate cells and portal fibroblasts, and recruit inflammatory cells, thereby contributing to the fibrogenic wound healing response. The availability of the Foxl1Cre mouse for the first time permits lineage tracking of progenitor cells during the differentiation to mature cholangiocytes and will allow us to begin to investigate the relationship between hepatic progenitor cells and reactive cholangiocytes.

The following materials and methods are provided to facilitate practice of the instant invention. They are particularly relevant to Example 2.

### Optimizing Isolation of Foxl1 Progenitors:

Non-parenchymal cells will be isolated from the liver following the protocols of Reid and Grompe and sorted via FACS. The clonogenic potential of the Foxl1Cre-labeled stem cells will be determined in vitro using serum-free culture on collagen matrix. Eight week old male mice of Foxl1-Cre; RosaYFP genotype will be fed a DDC containing diet (0.1% wt/wt) as performed previously (Sackett, S. D., et al., (2009) Hepatology 49, 920-929.). A preliminary experiment using immunofluorescent detection of the yellow fluorescent protein confirmed that the Foxl1-Cre expressing lineage can be labeled efficiently using RosaYFP mice (FIGS. 15 and 16).

### Isolation of Foxl1-Cre-Marked Cells:

For optimal preparation of the single cell suspension of non-parenchymal liver tissue required for FACS sorting, a step-wise digestion protocol will be followed (Wang, X., et al., (2003) Proc Natl Acad Sci USA 100 Suppl 1, 11881-11888.). Initially, the standard mouse liver perfusion and hepatocyte isolation will be performed to remove the bulk of the mature hepatocytes (Overturf K, et al., (1997) supra). The remaining solid liver tissue will be subjected to sequential digests (20 minutes each) with 2.5 mg/ml Collagenase D (Sigma-Aldrich), 10 mg/ml Collagenase D+ 10 mg/ml Pronase (Sigma-Aldrich), and a 10 minute digest with 0.05% Trypsin/EDTA (Promega). At each stage, dissociated cells will be collected by passage through a 40-μm cell strainer (BD Falcon) and stored on ice without further exposure to enzyme. Incompletely dissociated tissue/cell clusters from each step will be recovered from the strainer and exposed to the next digestion solution. Hepatocytes will be excluded from non-parenchymal cell (NPC) preparations by repeated low-speed (50 g) centrifugations.

### In Vitro Progenitor Assay:

FACS sorting will be performed. The feasibility of this approach has been validated. As shown in FIG. 15, after two weeks of DDC diet, approximately 0.2% of the nonparenchymal cells in the liver of Foxl1-Cre; RosaYFP mice were YFP positive. Sorted cells will be seeded at a density of 10³-10⁴ per cm² on rat Collagen I-coated Primaria (BD Falcon) tissue culture plastic ware with media changes every four days. Serum free-media with defined concentrations of free fatty acids will be prepared as described (MacDonald J M, et al., (2001) Ann N Y Acad Sci 944:334-343), and supplemented with 50 ng/ml of murine epidermal growth factor and 50 ng/ml of murine hepatocyte growth factor (Chemicon, Temecula, Calif.). Colonies, defined as organized circular clusters of at least 40 cells, will be scored on day 12.

### Identifying the Peak of Stem Cell Recovery:

Because a genetic mark produced in cells that expressed Foxl1-Cre at some point during their ontogeny is permanent, both hepatic stem cells and their descendants can be labeled by YFP in Foxl1-Cre; RosaYFP mice. In fact, it is this feature of the system that enabled establishment of the fact that Foxl1-Cre marks bipotential stem cells, as the descendants tracked to both hepatocytes and cholangiocytes (Sackett, S. D., et al., supra). The YFP-labeled cells obtained will represent a mixture of stem cells and their descendants. Thus, 100% efficiency in the clonogenic assay should not be expected.

This system will be utilized to determine the length of DDC treatment that yields the highest percentage of progenitor cells among the YFP population. YFP-positive cells will be isolated and the clonogenic assay on Foxl1-Cre; RosaYFP mice after 1, 3, 5, 7 and 14 days of DDC treatment will be performed. The time point with the highest percentage (not absolute number) of bipotential stem cells will be used.

Multiple systems have been employed for conditional cell ablation in mice. The most elegant is based on the fact that mice are not affected by diphtheria toxin treatment due to the absence of a cell surface receptor for the toxin. Thus, only when the human HBEGF gene, the receptor for diphtheria toxin, is expressed on the surface of the intended cells in transgenic mice do these cells become susceptible to ablation. The principle of this approach is outlined in FIG. 21.

As described, a bacterial artificial chromosome (BAC) transgene for the expression of the diphtheria toxin receptor in the Foxl1 lineage has been constructed, and transgenic founders obtained (FIGS. 18 and 19). This BAC transgene encompasses exactly the same regulatory elements that were used previously in the Foxl1-Cre transgenic mice (Sackett, S. D., et al., (2007) Genesis 45: 518-522; Sackett, S. D., et al., (2009) supra), thus it can target the same lineage.

### Lineage Tracing of DEN-Induced HCC:

It is necessary to label the hepatic progenitor cells briefly at the time the carcinogen is given. Using this strategy will eliminate the possibility that YFP expression in diethylnitrosamine (DEN) tumors resulted from late activation of the YFP transgene in response to DEN injury.

### Results

The co-staining studies indicate that portal fibroblasts detected by elastin stain are located in close proximity to Foxl1 progenitor cells in both BDL and DDC injury paradigms investigated (FIG. 12; and in many instances these cells appear to encircle Foxl1 positive cells. The juxtaposition of Foxl1-expressing progenitor cells and portal fibroblasts indicates the potential for paracrine signaling between these two cell populations. With the identification of Foxl1 as a marker of bipotential progenitors in BDL and DDC induced liver injury, and the development of a Foxl1-hDTR line for cell ablation and a tamoxifen inducible Foxl1-Cre mouse, it is possible to isolate hepatic progenitor cells and investigate their proliferative and differentiation potential both in vitro and in the transplant setting, to ablate progenitors during injury to evaluate their contribution to liver recovery, and to conditionally label these cells to study their potential role as liver cancer stem cells.

### Derivation of a Tamoxifen Inducible Foxl1-CreERT2 Mouse Strain

A new tamoxifen-inducible Foxl1-CreERT2 mouse strain will be utilized to assess Foxl1 characteristics. The tamoxifen-inducible Foxl1-Cre allows for precise timing and a controlled window of in vivo progenitor cell labeling. The question of whether Foxl1+ progenitors are hepatic cancer stem cells can be evaluated. Using this system, progenitors will be labeled for only two to three days, and then their contribution to cancer nodules evaluated months later. The existing Foxl1-Cre line cannot be used for this experiment, because a labeled cell in the cancer nodule could be marked at any time during the natural history of carcinogenesis.

Only through the use of the Foxl1-CreERT2 “pulse-chase” labeling approach can one determine if it was truly progenitors that contributed to the cancer, and not simply reactivation of the Foxl1 promoter at a later time. A similar approach has been employed previously in a seminal study to clarify progenitor/descendant relationships in the differentiation of pancreatic insulin-producing β-cells (Dor, Y., et al., (2004) Nature 429: 41-46). In the Foxl1-CreERT2 transgene, a fusion protein of the Cre recombinase with the ligand binding domain of the estrogen receptor is placed under the control of the regulatory elements of the Foxl1 locus. This fusion protein allows for tamoxifen-inducible deletion of loxP-flanked loci. The BAC transgene has been constructed and the first transgenic founders have been obtained (FIG. 13). Founders will be tested for efficient Cre activation by intraperitoneal injection of 2 mg tamoxifen and visualization of YFP in frozen sections of the GI tract where Foxl1 is expressed constitutively (Sackett, S. D., et al., (2007) supra). Founders that faithfully recapitulate Foxl1 expression will be expanded and Foxl1 CreERT2; RosaYFP mice will be utilized in further studies as described below.

Because of the increased ease of cell sorting compared to RosaLacZ mice, a Foxl1-Cre; RosaYFP mice was recently derived in which Cre-expressing cells are labeled by yellow fluorescent protein (YFP). When these mice are subjected to bile duct ligation or fed a DDC-containing diet to induce a ductular reaction, the Foxl1-marked hepatic stem cell lineage is strongly labeled (FIG. 14). The Foxl1-Cre; RosaYFP mice allow us to isolate the bipotential progenitor cell in Foxl1-Cre activated cells since Cre activates the expression of a fluorescent protein, allowing for fluorescent activated cell sorting. Sorted cells can then be cultured in vitro in attempts to expand their number, differentiated towards hepatocyte and cholangiocyte lineages, characterized using biochemical and molecular means, and transplanted into immunodeficient FAH^{-/-} mice. Approximately 0.1 to 0.2% of non-parenchymal cells in DDC-treated Foxl1 Cre; RosaYFP mice are YFP positive and thus represent progenitors and their immediate descendants.

### Isolation of Foxl1-Cre-Marked Cells

Foxl1-Cre marked cells will be isolated for in vitro clonogenic and differentiation assays and for cell replacement therapy in the FAH−/− mouse model. Preliminary studies have been performed to demonstrate that it is possible to isolate YFP+ cells from Foxl1Cre; RosaYFP DDC injured livers. For optimal preparation of the single cell suspension of non-parenchymal liver tissue required for FACS sorting, a step-wise digestion protocol was used, which is based on prior work (Wang, X., et al., (2003) supra). As shown in FIG. 15, after two weeks of DDC diet, approximately 0.1-0.2% of the nonparenchymal cells in the liver of Foxl1-Cre; RosaYFP mice were YFP positive. In addition, preliminary in vitro clonogenic assays with the sorted progenitor cells were performed. As shown in FIG. 16, the outgrowth of colonies of similar morphology as described by (Okabe, M., et al., (2009) Development 136: 1951-1960) was observed. No colonies grew when 100× the number of YFP-negative nonparenchymal cells were plated from the same mouse.

In order to begin the molecular characterization of the Foxl1-Cre/YFP marked progenitor population, gene expression of previously identified progenitor cells markers by quantitative RTPCR was analyzed. As is shown in FIG. 17, the YFP+ cells sorted from DDC-diet treated mice are highly enriched for EpCAM and Trop2 mRNAs demonstrating that the genetically marked progenitors are similar to progenitors previously only identified by immunostaining. These findings further strengthen the wisdom that Foxl1 indeed marks adult hepatic progenitors. Extending these studies, it will be possible to demonstrate that Foxl1 promotes cholangiocyte proliferation after BDL induced liver damages, as described in more detail in Example 3 and Sackett et al., J. Lab Invest., 2009 [in press].

The Foxl1-hDTR Model for Inducible Ablation of Adult Hepatic Progenitors

Mice are normally insensitive to diphtheria toxin (DT). Therefore, only cells in which the human diphtheria toxin receptor (hDTR) is expressed are ablated following toxin injection (FIG. 21). This system has been used successfully for the ablation of multiple cell-types previously (Buch, T., et al., (2005) Nat Methods 2: 419-426; Saito, M., et al., (2001) Nat Biotechnol 19: 746-750). A Foxl1-hDTR BAC transgene that directs expression of the hDTR to the Foxl1⁺ progenitor lineage has already been constructed, allowing for their ablation during the regenerative response, and transgenic founders have been obtained (FIGS. 18 and 19). Care was taken to first inactivate the Foxc2 gene, which is closely linked to Foxl1 (Kaestner, K. H., et al., (1996) Development 122: 1751-1758), because the function of Foxc2 is dosage dependent (Cederberg, A., et al., (2001) Cell 106: 563-573). This novel system will enable us to inducibly ablate the Foxl1 progenitor lineage in adult mice and analyze the recovery following several models of liver injury. This will allow assessment of the contribution of hepatic progenitors to liver homeostasis to be tested directly.

### Discussion

The first tools for the genetic labeling, sorting, and ablation of adult hepatic bipotential precursors have been developed. Herein it has been demonstrated that it is possible to isolate Foxl1⁺YFP⁺ progenitors by FACS sorting for in vitro studies of self-renewal and differentiation, and for in vivo studies of liver repopulation in the FAH^{-/-} model. The tools for ablation and conditional labeling of progenitors for the experiments have been developed. These novel approaches will provide valuable insights into hepatic progenitor biology and the origins of hepatocellular cancer.

These findings are important because the Foxl1-Cre transgene allow isolation of the bipotential progenitor cell by simply replacing the Rosa26R lacZ reporter with one in which Cre activates the expression of a fluorescent protein, allowing for fluorescent activated cell sorting (FIG. 15). Sorted cells can then be cultured in vitro in attempts to expand their number, the cells can be differentiated into cholangiocytes and hepatocytes, and characterized using biochemical and molecular means. These finding also allow evaluation of the proliferative and differentiation potential in vivo after transplantation into immunodeficient FAH null mice, as a first step towards cell replacement therapy in the liver. Also, one can evaluate the importance of progenitor activation in the liver's recovery from injury using targeted genetic ablation of these cells. Lastly, the Foxl1-Cre lineage tracing approach will enable one to address the question whether liver tumor cells are the descendants of oval cells as discussed in (Sell, S., et al., (2008), supra);).

In an effort to isolate adult hepatic progenitors and test their clonogenic and differentiation potential in vitro and in vivo, Foxl1-Cre marked adult hepatic progenitors can be expanded and differentiated towards mature hepatocytes in vitro and in vivo. Multiple paradigms have been established in rodents and humans for the activation of postulated hepatic stem cells, which differ in outcome with respect to currently available marker genes. The Foxl1-Cre BAC transgene has been shown to be activated in two of these paradigms, the bile duct ligation and DDC diet model. In both cases a “ductular reaction”, or proliferative activation of cells adjacent to the portal triad, ensues. Originally, genetic lineage tracing using the Foxl1-Cre transgene with the Rosa26R betagalactosidase reporter line was performed (Soriano, P. (1999) supra). Then co-immunostaining for specific markers of the cholangiocyte (cytokeratin 19, or CK19) and hepatocyte (HNF-4alpha) lineages to trace the fate of the genetically marked cells was performed. β-galactosidase positive cells acquired either HNF-4alpha or CK19 expression, consistent with the notion of a Foxl1-Cre progenitor that gave rise to both mature cell types.

Because of the increased ease of cell sorting, Foxl1-Cre;RosaYFP mice have been derived in which Cre-expressing cells will be labeled by yellow fluorescent protein. When these mice are subjected to bile duct ligation or fed a DDC-containing diet to induce a ductular reaction, the Foxl1-marked hepatic stem cell lineage is strongly labeled (FIG. 14). It has been further established that the Foxl1-Cre/YFP marked sorted cells express high levels of previously identified progenitor cell markers such as EpCAM and Trop2 (FIG. 17), again demonstrating the development of a powerful tool for the sorting of hepatic progenitor cells. Finally, the data indicates that the Foxl1-Cre/YFP cells can clonally expand in culture (FIG. 16).

Considering the in vitro differentiation of hepatic progenitors, genetic lineage tracing experiments have clearly demonstrated that Foxl1-Cre marked cells differentiate into both cholangiocytes and hepatocytes in vivo (Sackett, S. D., et al., (2009) supra). However, the proportion of differentiation was skewed towards the cholangiocyte lineage, possibly due to the cholestatic nature of injury in the BDL model. One can then determine if the differentiation of FACS sorted Foxl1-Cre; RosaYFP progenitors can be direct towards the hepatocyte lineage. Foxl1-Cre; RosaYFP labeled cells from mice treated for the optimal time period with DDC will be expanded in culture as described above before being subjected to in vitro differentiation protocols. Three different approaches will be utilized to differentiate hepatocytes These include (1) Following the protocol established by Weiss et al., progenitors will be placed in a sandwich culture onto collagen-coated glass cover slips, overlaid with 0.25 mg/ml Matrigel on the following day in medium containing dexamethasone, insulin-transferring-selenium, and EGF (Fougere-Deschatrette, C., et al., (2006) Stem Cells 24: 2098-2109); (2) Herrera et al. recently established that for the differentiation of a human adult liver population, culture in microgravity was essential in order to obtain cytochrome P450 expression and urea production. Therefore, one can culture Foxl1+ progenitors in the presence of HGF and FGF4 in a rotary cell culture system (Herrera, M. B., et al., (2006) Stem Cells 24: 2840-2850); and (3) Azuma et al. utilized culture of “immature hepatic cells” isolated from mouse nonparenchymal cell fractions in the presence of 1% DMSO and 100 nM dexamethasone, and achieved induction of several mature hepatocyte markers (Azuma, H., et al., (2003) Hepatology 37: 1385-1394).

It is possible to assess the following parameters in the differentiated cell populations by examining gene expression of hepatocyte-specific or enriched transcripts by real time qRT-PCR, for example, Albumin, Krt8, Krt18, TAT, G6 Pase, α-1AT, BSEP, glycogen synthetase, HNF4α, HNF1α, HNF6, Hex, C/EBPβ, C/EBPα. Positive control will be whole liver. Protein expression, immunohistochemistry and Western blot can be used for analysis for the Afp, Foxa2, albumin, HNF4α, α-1AT, BSEP, MRP2 genes; ultra-structural analysis (e.g. glycogen granules, canaliculus formation, gap and tight junctions, large polygonal cell shape, high number of mitochondria); and other functional assays such as albumin secretion, glycogen storage, ureagenesis, bilirubin metabolism, steroid metabolism, CYP function, lidocaine clearance. One can compare results with fresh primary hepatocytes. Using this comprehensive approach, one can determine the optimal conditions for differentiation of Foxl1-marked progenitors in culture, and also to which extent the mature differentiation state can be achieved in culture.

In terms of cell replacement therapy with Foxl1-marked progenitors in the FAH^{-/-}; Rag2^{-/-}; Il2rg^{-/-} (FRG) model, the most stringent assay for adult hepatic progenitor cells is to test their ability to repopulate an injured liver. Engraftment of hepatocytes into healthy mice has been shown to be very limited (Sancho-Bru, P., et al., (2009) supra). Large-scale repopulation of transplanted hepatocytes only occurs if the transplanted cells have a dramatic survival advantage over the host cells. Grompe et al. recently established FAH^{-/-}; Rag2^{-/-}, Il2rg^{-/-} (Azuma, H., et al., (2007) Nat Biotechnol 25: 903-910) mice that allow for efficient engraftment of mouse allografts and human xenografts. Use of this model will save the time of backcrossing the Foxl1Cre-RosaYFP mice to syngeneity with the FAH^{-/-} mouse.

The FRG model is deficient in the tyrosine catabolism enzyme fiunaryol-acetoacetate hydrolase (FAH). FAH null mice can be kept healthy by feeding with the protective drug NTBC (2-(2-nitro-4-trifluoromethylbenzoyl)-1,3-cyclohexanodione), which inhibits the proximal enzyme 4-hydroxyphenylpyruvate dioxygenase and prevents the accumulation of toxic metabolites in hepatocytes (FIG. 20). Only once NTBC is gradually removed after transplantation will resident hepatocytes die, leaving both space and growth advantage for transplanted cells.

Azuma et al. (2007) reported that human hepatocyte engraftment in FRG mice was obtained only if recipient mice first received treatment with an adenovirus expressing the secreted form of uPA. In contrast, mouse hepatocytes repopulated livers of FRG mice regardless of uPA expression. The following general transplantation protocol will be employed: adult (6-15 week old) FRG mice raised with NTBC therapy will be used. Control groups will receive one million mouse hepatocytes intrasplenically. Experimental groups will receive 10,000 Foxl1 Cre; RosaYFP positive cells, plus varying numbers of mouse hepatocytes, also intrasplenically. In the experimental groups, one million, 500,000, 250,000, 100,000, 50,000 and zero hepatocytes will be used to complement the injected progenitors. After transplantation, NTBC will be gradually withdrawn over 7 days. NTBC will be reinstated two weeks later for five days and then removed permanently (Azuma, H., et al., (2007) supra). Control: transplantation of 1,000,000 hepatocytes intrasplenically (is curative and leads to ˜25% engraftment). Experimental: Limited hepatocytes (500K, 200K, 100K, 50K, OK) plus 10K sorted progenitors.

Mice will be monitored for weight weekly and livers harvested for analysis three months post-transplant. Final analysis will be done at necropsy with analysis of liver sections for engraftment of transplanted progenitor cells, which will be detected by YFP immunofluorescence as described above (FIG. 14). Dual immunofluorescence staining for YFP and HNF4alpha will also be performed to determine what fraction of the transplanted cells have become hepatocytes. To confirm engraftment and differentiation into functional hepatocytes, immunostaining for FAH and determination of FAH enzyme activity in liver will be performed. In order to exclude the possibility of cell fusion as the reason for engraftment, sex-mismatched grafts and recipients will be utilized. Recipients and donor hepatocytes will be female, and donor progenitors male, and sex chromosomes in grafts visualized using immunostaining followed by deconvolution confocal microscopy. In addition, it will be possible to determine karyotypes of FACS sorted YFP positive cells from the recipient liver.

It will also be interesting to determine the proliferative capacity of the engrafted progenitors. For this purpose, long-term labeling of proliferating with the thymidine analog EdU (5-ethynyl-2′-deoxyuridine) in the drinking water will be employed. By staining of liver sections for EdU and YFP at the same time, one can determine what fraction of the transplanted progenitors have undergone S-phase, and presumably proliferated, in the labeling interval (Salic, A., et al., (2008) Proc Natl Acad Sci USA 105: 2415-2420). By varying the time of EdU exposure, one can estimate the rate of proliferation of the progenitor-derived cells at varying times posttransplant. By employing two thymidine analogues successively, one can also determine if progenitor derived hepatocytes have divided multiple times (Teta, M., et al., (2007) Dev Cell 12: 817-826).

Subsequently, one can assess the contribution of the Foxl1-positive stem cell lineage to the proliferative response following liver injury in light of establishing that the Foxl1-Cre transgene marks bipotential hepatic progenitor cells. One can specifically ablate the Foxl1+ lineage from the injured liver using the Foxl1-hDTR (human diphtheria toxin receptor) transgenic mouse which has been developed. In this animal, application of diphtheria toxin will destroy all Foxl1+ cells, without affecting the remainder of the organism. Ablation of the entire Foxl1 progenitor cell lineage will likely be associated with severe hepatic morbidity following either cholestatic or genotoxic liver injury.

A further rationale for these studies comes from the recent findings that mice deficient for the Foxl1 gene itself exhibit reduced biliary proliferation and increased necrosis in the bile duct ligation model of cholestatic liver injury (Sackett, S. D., et al., J Lab Invest 2009; [in press]). These findings indicate that Foxl1 activity in hepatic progenitor cells is required in part for cholangiocyte differentiation and proliferation. However, the Foxl1 null model does not address the question of the contribution of adult progenitors to the liver's recovery from injury, because the progenitor cells are still present in these mice. The complete ablation of the Foxl1+ progenitor lineage can only be accomplished using the novel model described herein.

Foxl1-DTR mice will be assessed using two paradigms of liver injury, bile duct ligation (BDL) and treatment with a DDC supplemented diet, as performed previously. Cohorts of ten 8-week old mice each will be treated with 100 ng diphtheria toxin i.p. injections two days before and five days after the onset of either treatment to ensure that all Foxl1⁺ progenitors will be ablated (Buch, T., et al., (2005) supra). Foxl1-DTR transgene negative littermates will serve as control, as well as saline-injected Foxl1-DTR mice.

The extent of injury will be monitored by multiple criteria as performed previously. Weight and survival will be assessed daily, and mice will be sacrificed after five days and two weeks, corresponding to acute and chronic injury time points respectively. AST, ALT, necrotic areas, CK19 staining, and CK19/PCNA double positive cells will be examined. Fibrosis will be evaluated by computer assisted quantitative area analysis of Sirius red and trichrome stained tissue sections and hydroxyproline assay (Sackett, S.D. (2009) Hepatology 49: 920-929). Apoptosis will be assessed by TUNEL assay (ApopTAG, Millipore). Expression of Wnt family members including Wnt3a and Wnt7b was reduced in Foxl1^{-/-} livers post-BDL(. Therefore, quantitative real-time PCR will be used to investigate whether expression of canonical Wnt genes is even more dramatically reduced in livers in which Foxl1⁺ progenitors have been ablated.

Although hepatic necrosis was greater in Foxl1^{-/-} livers relative to controls two weeks post-BDL, hepatocyte proliferation was decreased in Foxl1^{-/-} livers at earlier time points post-BDL when necrotic injury was similar in Foxl1^{-/-} and control livers. This finding led to investigation of whether Foxl1 activation of Wnt3a and/or Wnt7b in progenitors may stimulate hepatocyte proliferation through paracrine activation of the β-catenin signaling pathway. Thus, without being bound by theory, hepatocyte proliferation will be inhibited to an even greater extent in Foxl1⁺ progenitor ablated livers post-BDL. This can be quantified by percent Ki67⁺/total hepatocytes at both 5d (acute injury) and 14d BDL (chronic injury) time points.

The concept of a hepatic cancer stem cell was proposed several decades ago, but thus far is only supported by correlative evidence. That is to say, liver carcinogens frequently first cause a ductular reaction, or the appearance of multiple small cells (oval cells) in the portal triad, before they cause cancer. To assess the role of the Foxl1-positive hepatic stem cell lineage in hepatocellular carcinoma, it will be useful to test the idea that chemically induced liver cancer is derived from hepatic stem cells by genetically labeling the Foxl1+ progenitor before treating mice with DEN (diethylnitrosamine) a carcinogen that is known to stimulate oval cell proliferation at an early stage of liver injury (He, X. Y., et al., (1994) Pathology 26: 154-160). If indeed hepatocellular carcinomas are derived from liver stem cells, then the cancerous nodules should be YFP positive.

In a complementary study, one can adapt a novel paradigm established earlier this year to determine the cell-of-origin for intestinal cancer to the liver. Barker et al. used a Cre line specific for the intestinal stem cells to ablate the adenomatous polyposis coli (APC) gene, a gene well known to cause colorectal cancer in people, in intestinal crypts (Barker, N., et al., (2009) Nature 457: 608-611.). These mutant mice rapidly developed macroscopic adenomas. When, on the other hand, they employed a Cre line to delete the APC gene in transit amplifying cells, growth of microadenomas stalled quickly, and only rare large adenomas occurred. Similar findings were reported by Zhu and colleagues using a Prom1/CD133 driven Cre (Zhu, L., et al., (2009) Nature 457: 603-607). These genetic studies lend strong support to the cancer stem cell concept for intestinal cancer.

In the mouse liver, genetic ablation of the APC gene in mature hepatocytes only rarely leads to the formation of hepatocellular carcinoma (HCC) (Colnot, S., et al., (2004) Proc Natl Acad Sci USA 101: 17216-17221.). If HCC is indeed derived from hepatic progenitor cells, then one could expect a much higher frequency of HCC when one deletes the APC gene using the Foxl1CreERT2 transgene.

In regard to lineage tracing of DEN induced HCC, one can utilize the new tamoxifen-inducible Foxl1-CreERT2 mouse strain described herein. In this transgene, a fusion protein of the Cre recombinase with the ligand-binding domain of the estrogen receptor was placed under the control of the regulatory elements of the Foxl1 locus (FIG. 22). This fusion protein allows for tamoxifen-inducible deletion of loxP-flanked loci. The BAC transgene has been constructed and the first transgenic founders have been obtained (see FIG. 13). Once transgenic lines have been established, these will be bred to the RosaYFP strain, and progenitor cells labeled by a short pulse of tamoxifen PRIOR to the initiation of the DEN protocol. Because the contribution of estrogens markedly reduces in the incidence of hepatocellular carcinoma development in females (Naugler, W. E., et al., (2007) Science 317: 121-124.), only male mice will be used for these studies.

In order to activate the Foxl1-CreERT2 in pups at 7 days of age, a tamoxifen pellet will be implanted subcutaneously in lactating females. This technique has been used successfully to effect tamoxifen induced gene deletion in pups via transfer of tamoxifen in breast milk (Leone, D. P., et al., (2003) Mol Cell Neurosci 22: 430-440; Gao, N., et al., (2008) Genes Dev 22: 3435-3448). Ten fifteen-day old male Foxl1-CreERT2:Rosa YFP and RosaYFP (Cre-negative) mice each will subsequently receive a single intraperitoneal 25 mg/kg dose of DEN (Sigma-Aldrich), a protocol that reliably induces multiple hepatocellular tumors in wild type mouse livers at 8 months of age (Pierce, R. H., et al., (2002) Am J Pathol 160: 1555-1560).

Five animals per group will be sacrificed at 4 and 8 months of age for analysis of YFP positive cells in preneoplastic (4 months) and neoplastic lesions (8 months). At the time of liver harvest, livers will be removed, the surface visually inspected for tumor burden, weighed and photographed at the time of harvest. The tumors will then be fixed overnight in paraformaldehyde for histological analysis of tumors and preneoplastic lesions. Sections will be stained with rabbit anti-GFP (Abcam ab290), using the Vector ABC kit for immunodetection to establish whether neoplastic hepatocytes express YFP. Fractional labeling will be determined by co-staining the nuclei of the lesions with DAPI, and counting the double-stained versus total tumor cells.

Recent genetic approaches have clarified the cell of origin for intestinal cancer in mice, demonstrating clearly that ablation of the Apc gene in intestinal stem cells is much more tumorigenic than deletion of the same tumor suppressor gene in the transit amplifying cells that are derived from the stem cell. These data provide strong evidence for the importance of stem cells in CRC. Now one can perform the analogous experiments for hepatocellular carcinoma.

The loxP flanked Apc gene will be obtained (official allele designation Apctm2.1Cip; hereinafter “ApcloxP”) (Colnot, S., et al., (2004) supra) and crossed to both the Foxl1-CreERT2 line, and to the Albumin-CreERT2 strain (provided by Dr. Chambon), which is already in the colony (Schuler, M., et al., (2004) Genesis 39: 167-172.). While the former will target hepatic progenitors for gene ablation, the latter is active only in mature hepatocytes. Groups of twenty 2-months old males each for the following genotypes will be used for the analysis: (1) ApcloxP/loxP; Foxl1-CreERT2; and (2) ApcloxP/loxP; Albumin-CreERT2.

In order to induce Foxl1 gene activation, all mice will be treated for seven days with the DDC diet as described herein. Ten mice in each group will receive three daily injections of tamoxifen on day 5, 6 and 7 of the DDC diet, while the other ten mice in each group will receive vehicle. Mice will be harvested for analysis six months later. Livers will be analyzed for tumor formation macroscopically on 5 mm think liver slices after hematoxylin/eosin (H&E) and reticulin stained liver sections. Microscopic tumor sections, five per mouse and spaced at 0.2 mm intervals, will be counted on H&E stained sections. Apc ablation will be scored by nuclear beta-catenin staining (Colnot, S., et al., (2004) supra). Given that DDC diet has been shown to accelerate tumor formation at least in some transgenic mouse models (Beer et al., 2008), additional groups of mice will be analyzed at shorter intervals after Apc ablation.

Without being bound by theory, it is likely that: (1) the Foxl1-YFP expressing cells will be highly enriched for hepatic progenitor cells; (2) it will be possible to differentiate the Foxl1-YFP progenitors towards functional hepatocytes; (3) it will be possible to determine the epigenetic profile of freshly isolated progenitors and in vitro differentiated hepatocytes to those freshly isolated from the liver; (4) it will be possible to functionally repopulate the liver of FRG mice with substantially smaller number of cells than are currently required with mature hepatocytes dues to the higher proliferative capacity of the progenitors; (5) ablation of Foxl1 expressing cells will be associated with increased liver injury which may be manifest by decreased biliary proliferation and increased hepatic necrosis in the BDL model and reduced oval cell proliferation in the DDC treatment group; and (6) these studies will provide an answer to the question whether hepatocellular carcinoma is derived from resident hepatic progenitor cells marked by Foxl1, or whether differentiated hepatic epithelial cells can also contribute to liver cancer.

### Example 3

Foxl1 uniquely marks bipotential hepatic progenitor cells, and both Foxl1-Cre and tamoxifen-inducible Foxl1-CreERT2 mouse strains enable both gain- and loss-of function studies in vivo through the targeted ablation of components of these signaling pathways. Using these tools, one can dissect the autocrine and paracrine signaling events that contribute to progenitor cell recruitment, differentiation and proliferation as well as paracrine effects on fibrogenesis during tissue repair in response to liver injury. Understanding the biology of hepatic progenitor cells will have a wide-ranging impact on the development of therapeutics for acute liver failure, chronic cirrhotic liver disease and hepatocellular carcinoma. Investigation of the contribution of signaling pathways for liver progenitor cell function during liver repair is described hereinbelow.

We have designed studies to elucidate the molecular mechanisms that direct the self-renewal, proliferation and differentiation of hepatic progenitor cells during the reparative response to liver injury. Without being bound by theory, a signaling hierarchy exists in which the winged helix factor Foxl1 mediates hedgehog to wnt/β-catenin signaling necessary for progenitor cell renewal and proliferation. Notch likely acts downstream of wnt/β-catenin, and is necessary for progenitor cell differentiation towards the biliary lineage and may also be required for biliary tubule morphogenesis. Understanding the biology of hepatic progenitor cells will have a wide-ranging impact on the development of therapeutics for acute liver failure, chronic cirrhotic liver disease and hepatocellular carcinoma.

The hepatic stem cell niche is discussed in Example 2, and this example is concerned, among other things, with reactivation of developmental signaling pathways in the hepatic stem cell niche. Little is known at present regarding either the signals that maintain hepatic stem cell self-renewal in the quiescent liver or those that signal the stem cell to proliferate and differentiate towards a particular cell fate lineage in response to various types of liver injury. The finding that hedgehog, wnt and Notch signaling pathways function in concert to regulate stem cell proliferation and cell fate decisions in other tissues has led investigators to explore the contribution of these signal pathways to stem cell expansion and differentiation in the liver. Other signaling pathways that have been implicated in hepatic stem cell homeostasis include TGF-β (Nguyen L N, et al., (2007) Hepatology 45:31-41; Tang Y, et al., (2008) Proc Natl Acad Sci USA 105:2445-2450 Clark J B, et al., (2005) Biochem Biophys Res Commun 329:337-344) and JAK/STAT (Matthews V B, et al., (2004) Wound Repair Regen 12:650-656; Yeoh G C, et al., (2007) Hepatology 45:486-494).

Recent work by Diehl and colleagues in both experimental models and patient livers has established that hedgehog signals are reactivated in various types of liver injury in adults (Omenetti A, et al., (2008) Am J Physiol Gastrointest Liver Physiol 294:G595-598). In the quiescent state, Patched (Ptc), a component of the hedgehog receptor complex, prevents activation of Smoothened (Smo). Activation of the hedgehog pathway is initiated by binding of a hedgehog ligand such as sonic hedgehog (shh) to Ptc, which relieves suppression of Smo, followed by activation and nuclear translocation of the Gli1 transcription factor. Gli1 is then able to activate the transcription of itself as well as Gli2, another transcriptional activator (FIG. 23). The transcriptional repressor Gli3 and inhibitory receptor subunit Ptc are also induced by hedgehog and function as part of a negative feedback loop to constrain hedgehog signaling. Using a co-culture system of immature cholangiocytes and hepatic stellate cells, Diehl et al. demonstrated that both cell populations are able to both respond to and produce hedgehog ligands and that the presence of these ligands is critical for survival, proliferation and migration of hepatic progenitor cells in culture (Omenetti A, et al., (2008)supra). In a gain-of-function experiment of hedgehog activation, mice heterozygous for Ptc (Ptc^{+/-}) spontaneously develop expanded ductular reactions and increased fibrosis relative to littermate controls (Omenetti A, et al., (2007) Lab Invest 87:499-514). While this study indicates that constitutive activation of hedgehog signals in the liver is associated with progenitor and/or cholangiocyte proliferation and fibrosis, the specific regulatory pathways and cell types responsible for progenitor cell expansion and fibrogenesis are unknown. With the availability of the Foxl1-Cre mouse one can now selectively activate or inhibit hedgehog in hepatic progenitors and analyze the role of this pathway in progenitors during liver injury.

The canonical wnt/β-catenin pathway plays an important role in regulating the balance between sternness and differentiation in adult including intestine, skin and mammary gland (reviewed in Fodde R, et al., (2007) Curr Opin Cell Biol 19:150-158). In the liver, this signaling pathway is essential for regulation of development (Decaens T, et al., (2008) Hepatology 47:247-258), regeneration and protection against oxidative stress and misregulation of the wnt/β-catenin pathways is linked to the pathogenesis of hepatitis and hepatocellular carcinoma (reviewed in Thompson M D, et al., (2007) Hepatology 45:1298-1305). In the absence of wnt, intracellular levels of β-catenin are regulated by a complex of proteins including GSK3β (glycogen synthase kinase-3β), CK1 (casein kinase 1), and the scaffold proteins APC (adenomatous polyposis coli), Axin 1, and Axin 2 that direct β-catenin to the ubiquitin-mediated degradation pathway. Wnt ligand engagement to the frizzled receptor in concert with co-activator receptors LRP (low-density lipoprotein receptor-related proteins) results in the inhibition of the degradation complex, stabilization of β-catenin and nuclear translocation where it binds to members of the TCF/LEF transcription factor family and activates gene transcription (FIG. 24). Among β-catenin target genes are those encoding proteins involved in a wide range of processes including stemness (nanog, oct4), cell proliferation (c-myc, cyclin D), angiogenesis (VEGF), and epithelial-mesenchymal transition (snail, twist). Wnts also signal through β-catenin independent mechanisms such as activation of the ROR and RYK tyrosine kinase receptors (Nusse R. (2008) Cell Res 18:523-527).

Several recent gain- and loss-of-function studies in rodents have established that wnt/β-catenin signals play an important role in hepatic stem cell self-renewal and expansion. Oval cell expansion is decreased when β-catenin signaling has been inhibited in both hepatocytes and cholangiocytes (Apte U, et al., (2008) Hepatology 47:288-295) and forced expression of a constitutively active β-catenin mutant in isolated hepatic progenitor cells confers increased self-renewal behavior in vitro and results in the development of hepatic stem cell tumors in a xenotransplantation model (Chiba T, et al., (2007) Gastroenterology 133:937-950). While these studies provide compelling evidence for the importance of wnt/β-catenin signaling in hepatic progenitor cells, many questions remain to be addressed including identification of the upstream activator of the wnt/β-catenin pathway during liver injury and a detailed knowledge of autocrine and paracrine signals that regulate progenitor cell expansion. The expression of a large number of wnts, receptors and co-ligands in multiple liver cell populations poses a significant challenge to understanding these pathways in vivo (Zeng G, et al., (2007) Hepatology 45:195-204.).

Foxl1 has been identified as an important regulator of the β-catenin mediated cholangiocyte proliferation in the bile duct ligation model of liver injury. The reduced expression of canonical Wnt genes in Foxl1^{-/-} mice indicates that Foxl1 regulates β-catenin by an autocrine mechanism. In the intestine, Foxl1 is activated by the sonic hedgehog pathway (Madison B B, et al., (2009) J Biol Chem 284:5936-5944). These findings lead to the idea that in the liver, Foxl1 is a downstream target of hedgehog signals required for activation of wnt/β-catenin regulation of self-renewal and proliferation. One can investigate both the contribution of wnt/β-catenin signals for hepatic progenitor cell renewal and expansion and the specific role of Foxl1 in this process.

Notch signaling is highly conserved throughout evolution and relies on ligand-receptor interactions that are dependent upon cell-cell contacts (reviewed in Kopan R, et al., (200) Cell 137:216-233). Four Notch receptors (Notch1-4), multiple ligands (including Jagged1 and 2, DLL1, 3, 4) and co-receptors exist in mammals. Notch 2 is the principal receptor expressed in the developing liver (Tchorz J S, et al., (2009) Hepatology; ePUB.). Activation of Notch receptors by their ligands leads to proteolytic release of the Notch intracellular domain (NICD) that then translocates to the nucleus where it binds to CSURBPjκ. This interaction results in activation of Notch target genes including members of the Hes (Hairy enhancer of split) and Hey gene families, helix-loop-helix proteins that function as transcriptional repressors. In the absence of NICD binding, RBPjκ inhibits target genes by recruiting a repressor complex. This repressor complex is replaced by a co-activator complex in response to NICD binding to RBPjκ (FIG. 25). As a result of receptor proteolysis, an individual receptor can only be used once. Therefore, receptor and ligand availability represents an important mechanism for regulating Notch signaling. Post-translational modifications of Notch receptors and control of intracellular trafficking provide additional levels of regulation (Kopan R, et al., (2009) supra).

Evidence for activation of Notch signaling in the injured adult liver comes from studies showing that hepatocyte proliferation (Kohler C, et al., (2004) Hepatology 39:1056-1065) and endothelial cell viability (Wang L, et al., (2009) Hepatology 49:268-277) in the regenerating liver are Notch dependent. Mutations have been identified in the Notch ligand Jagged 1 (Oda T, et al., (1997) Nat Genet 16:235-242; Li L, et al., (1997) Nat Genet 16:243-251) or Notch-2 receptor (McDaniell R, et al., (2006) Am J Hum Genet 79:169-173) in patients with Alagille syndrome, a cholangiopathy characterized by bile duct paucity. Findings from Notch pathway gain- and loss-of function experimental models indicate an essential role for the Notch signaling pathway in biliary cell fate determination and bile duct morphogenesis during development (Zong Y, et al., (2009) Development 136:1727-1739; Geisler F, et al., (2008) Hepatology 48:607-616; Lozier J, et al., (2008) PLoS ONE3:e 1851) and postnatal biliary proliferation (Loomes K M, et al., (2007) Hepatology 45:323-330). Forced expression of the Notch modifiers Lunatic, Radical and Manic Fringe results in bile duct proliferation in the postnatal period, indicating that Notch may be involved in remodeling of bile ducts in response to hepatic injury (Ryan M J, et al., (2008) Hepatology 48:1989-1997). Expression of Jagged and Notch is increased in patients with chronic liver disease (Nijjar S S, et al., (2001) Hepatology 34:1184-1192). Notch may also regulate trans-differentiation of hepatocytes to cholangiocytes by controlling the expression of hepatocyte-enriched transcription factors (Tanimizu N, et al., (2004) J Cell Sci 117:3165-3174; Nishikawa Y, et al., (2005) Am J Pathol 166:1077-1088). Ductular cells in Alagille Syndrome patient livers fail to express markers of differentiated cholangiocytes, indicating that Notch signaling is essential for hepatic progenitor cell differentiation to mature cholangiocytes during postnatal liver repair (Fabris L, et al., (2007) Am J Pathol 171:641-653). The slow progression of fibrosis in patients with Alagille Syndrome in comparison to the rapid fibrosis progression in another cholangiopathy, Biliary Atresia, may be directly related to the inability of intermediate hepatobiliary progenitor cells in Alagille Syndrome livers to become reactive cholangiocytes. Reactive cholangiocytes in the livers of patients with Biliary Atresia secrete chemotactic factors that recruit and activate myofibroblast and inflammatory cells to the site of injury; thus, the failure of cholangiocyte precursors to differentiate to reactive cholangiocytes in the livers of patients with Alagille Syndrome may explain the differences in fibrosis in the two diseases.

Livers of patients with chronic liver disease and experimental models associated with severe inflammation and parenchymal necrosis contain increased numbers of “intermediate” hepatocytes, indicating that signals directing differentiation of progenitors to the hepatocyte lineage may be activated in response to significant hepatocyte death (Roskams T, et al., (2003) Am J Pathol 163:1301-1311; Jung Y, et al., (2008) Gastroenterology 134:1532-1543). The signals that direct progenitors to differentiate into hepatocytes are unknown; however, inhibition of Notch signaling would be predicted to redirect progenitors away from the cholangiocyte lineage and enhance differentiation of progenitors to the hepatocytes. Thus, inhibition of Notch signaling in patients in whom repair is dependent upon hepatocytes could prove to be beneficial. One can investigate the role of Notch signaling for cholangiocyte lineage allocation and for biliary morphogenesis by assessing differences in lineage allocation and bile duct morphogenesis in a Notch loss-of-function genetic mouse model.

The following materials and methods are provided to facilitate practice of the instant invention. They are particularly relevant to Example 3.

### Conditional Deletion of Hedgehog Signaling Proteins in Hepatic Progenitor Cells During BDL and DDC Induced Hepatic Injury

Foxl1-CreERT2 mice will be crossed with smo^{loxP/loxP} or ptc^{loxP/loxP} mouse strain using the following crosses to obtain Foxl1CreERT2;smo^{loxP/loxP} or Foxl1CreERT2;ptc^{loxP/loxP} and respective controls. The mating strategy for the deletion of smoothened is outlined in FIG. 35, an analogous strategy will be employed for patched ablation. A summary of the experimental and control groups and effect of conditional deletion of smo and ptc for each strain is summarized in Table I.

TABLE I

| | | Constitutive |
|---|---|---|
| | Inhibition of hedgehog | activation of |
| | signaling | hedgehog signaling |
| Experimental | Foxl1CreERT2; | Foxl1CreERT2; |
| | smo^{loxP/loxP}; RosaYFP | ptc^{loxP/loxP}; RosaYFP |
| Control | smo^{loxP/loxP}; RosaYFP | ptc^{loxP/loxP}; RosaYFP |

Male mice only will be used for all these studies based on previous experience indicating that tamoxifen treatment is associated with glucose intolerance in female mice (Gao N, et al., (2007) Cell Metab 6:267-279). In order to activate the CreERT2 enzyme, a tamoxifen pellet will be implanted subcutaneously at 7 weeks of age. Three weeks later each genetic strain shown in Table II will undergo BDL or sham surgery and livers will be harvested 1, 3, 7, and 21 days after surgery. A similar treatment strategy will be used for DDC-diet with livers harvested 1, 3 7, and 21 days after DDC or chow diet. Five mice will be used for each time point and experimental condition. The details of the treatment strategy are outlined in Table II. The treatment groups are shown for smo conditional deletion using the Foxl1CreERT2;smo^{loxP/loxP} transgenic strain (Group (1)) and corresponding Cre negative controls (Group (2)). An additional control will be to test smo^{loxP/loxP}:RosaYFP mice without tamoxifen treatment (Group (3)) to exclude any potential effects on BDL or DDC injury or recovery due to the tamoxifen treatment itself. The analogous treatment groups will be used for Foxl1CreERT2:Ptc^{loxP/loxP}. These time points have been selected so that one can assess differences in both acute and chronic injury. For analysis of gene expression in isolated progenitor cells, all experimental and control mice will be bred to heterozygosity for the RosaYFP locus. For these studies, Groups (4) and (5)) will be used as controls.

TABLE II

| | | | Sham surgery |
|---|---|---|---|
| | Tamoxifen | BDL or | (For DDC control, |
| Genotype | tx | DDC diet | 21 day only) |
| (1) Foxl1CreERT2; | + | 1, 3, 7, 21 | 1, 3, 7, 21 |
| smo^{loxP/loxP;} RosaYFP | | days | days |
| (2) smo^{loxP/loxP}; | + | 1, 3, 7, 21 | 1, 3, 7, 21 |
| RosaYFP | | days | days |
| (3) smo^{loxP/loxP}; | − | 1, 3, 7, 21 | |
| RosaYFP | | days | |
| (4) Foxl1CreERT2; | + | 1, 3, 7, 21 | 1, 3, 7, 21 |
| RosaYFP | | days | days |
| (5) Foxl1CreERT2; | − | 1, 3, 7, 21 | |
| RosaYFP | | days | |

### Conditional Deletion of β-Catenin in Foxl1⁺ Reactive Cholangiocytes and Bipotential Progenitor Cells During BDL and DDC Induced Hepatic Injury

Cttnb1^{loxP/loxP} mice will be obtained from Jackson laboratories. See the world wide web at jaxmice.jax.org/strain/004152.html. Foxl1-CreERT2 mice will be crossed with the cttnb1^{loxP/loxP} mouse strain using the same breeding strategy as outlined for Foxl1CreERT2;smo^{loxP/loxP};RosaYFP shown in FIG. 33 to obtain Foxl1CreERT2; cttnb1^{loxP/loxP};RosaYFP and cttnb1^{loxP/loxP};RosaYFP controls. The details of Cre-mediated gene ablation, treatment groups, and time points post-BDL or DDC diet paradigms will be the same as those described for conditional deletion of smo.

### Conditional Deletion of Rbpj^{loxP/loxP} in Foxl1⁺ Reactive Cholangiocytes and Bipotential Progenitor Cells During BDL and DDC Induced Hepatic Injury:

Notch signaling will be inhibited in hepatic progenitors during liver injury by conditional deletion of Rbpj, a DNA binding protein that is required for Notch signaling (Kopan R, et al., (2009) Cell 137:216-233). Rbpj^{loxP/loxP} mice will be obtained from RIKEN Bioresource Center, Tsukuba, Japan and will be crossed with Foxl1-CreERT2 miceusing the same mating scheme outlined above (FIG. 18) to obtain Foxl1CreERT2;Rbpj^{loxP/loxP};RosaYFP mice. In addition to the Cre negative control, Foxl1 CreERT2;RosaYFP mice will also be bred for studies in which differences in expression of specific lineage markers in vivo and following YFP⁺ cell isolation is analyzed. The details of Cre inactivation, treatment groups and time points post-BDL or DDC diet will be the same as those described for conditional deletion of smo. Treatment groups and time points examined post-BDL and DDC diet are shown in Table IV. Foxl1-CreERT2; Rbjp^{loxP/loxP};RosaYFP and Cre-controls will undergo either BDL or receive DDC supplemented diet.

TABLE IV

| | | | Sham surgery |
|---|---|---|---|
| | | | POL) (For DDC |
| | Tamoxifen | BDL or | control, chow |
| Genotype | tx | DDC diet | diet 21 days only) |
| Notch loss of function | | | |
| experimental | | | |
| and control groups: | | | |
| (1) Foxl1CreERT2; | + | 1, 3, 7, 21 | 1, 3, 7, 21 |
| Rbpj^{loxP/loxP;} RosaYFP | | days | days |
| (2) Foxl1CreERT2; RosaYFP | + | 1, 3, 7, 21 | 1, 3, 7, 21 |
| | | days | days |
| (3) Rbpj^{loxP/loxP}; RosaYFP | + | 1, 3, 7, 21 | 1, 3, 7, 21 |
| | | days | days |
| (4) Rbpj^{loxP/loxP}; RosaYFP | − | 1, 3, 7, 21 | |
| | | days | |

### Analysis of Injury and Hepatobiliary Lineage Allocation

One hour prior to animal sacrifice, mice will receive an intraperitoneal injection of BrdU 50 mg/kg. At the time of liver harvest, serum will be obtained and analyzed for AST, ALT and Total Bilirubin (Anilytics, Gaithersburg, Md.). Livers will be fixed in paraformaldehyde and either paraffin embedded or sucrose for cryopreservation. Sections will be examined for differences in necrosis, inflammation (H and E) and fibrotic injury (trichrome, Sirius red). Portions of each liver will be frozen for subsequent RNA and protein expression analyses as indicated by phenotypic changes observed on liver tissue sections. Loss of Hes1 immunostaining in YFP⁺ cells will be used to confirm the inhibition of Notch signaling which could occur in the Foxl1-CreERT2Rbpj^{loxP/loxP};RosaYFP BDL livers. CK19 and BrdU immunostaining will be used to quantify cholangiocyte/progenitor cell proliferation and YFP staining will be used to assess Cre activity. Cholangiocyte area will be quantified using IP lab image morphometry software of CK19 stained cholangiocytes (Sackett S D, et al., (2009) supra). CK7/CK19 double staining will also be performed in order to detect immature CK7⁺CK19⁻ bile ductular cells that could be increased in the absence of Notch signals. TUNEL assay will be performed to assess for differences in cholangiocyte and hepatocyte apoptosis. Immunostaining will be performed to quantify YFP⁺, YFP⁺CK7⁺, YFP⁺CK19⁺ and YFP⁺HNF-4α⁺ cells to determine if there is a shift in lineage allocation of YFP⁺ progenitor cells towards the hepatocytic lineage, or alternatively, as indicated by the findings from patients with Alagille Syndrome, that in the absence of Notch signals, YFP⁺CK19⁻HNF-4α⁻ progenitor cells are unable to differentiate towards either cholangiocytic or hep-atocytic lineages. In this case, one could expect to see an increase in the percentage of YFP⁺CK19⁻HNF-4α⁻ cells relative to the percentage of this cell population in Foxl1CreERT;RosaYFP (control) injured livers. In patients with Alagille Syndrome, ductular cells resembling cholangiocytes are CK7⁺ but do not express either CK19 or HNF-1β (Fabris L, et al., 2007, supra). CK7 and HNF-1β expression in Notch inhibited BDL and DDC injured livers and controls will be quantified to investigate the notion that a similar lineage block has occurred in the mice. In Alagille Syndrome livers, the diffuse pattern of intermediate cells localized within the parenchyma can be contrasted with the periportal location of intermediate cells adjacent to reactive ductular cells in livers from patients with biliary atresia, a cholangiopathy not associated with impaired Notch signaling. Therefore, the location and distribution of YFP⁺ signal may indicate impaired ability of these cells to form ductules will be investigated. Another possibility is that in the absence of Notch signals in progenitor cells, hepatocytes transdifferentiate to become cholangiocytes directly; a model that has been proposed based on lineage analyses of Alagille syndrome livers (Fabris L, et al., (2000) Am J Pathol 156:1599-1612; Van Eyken P, et al., (1988) Hepatology 8:1586-1595; Haruna Y, et al., (1996) Hepatology 23:476-481). In this case, one could expect that cells within ductular reactions will be negative for YFP. To visualize the impact of Notch signal inhibition on ductular morphogenesis, 3-D reconstruction and corrosion casts of the biliary tree will be performed to evaluate the potential effects of Notch signaling on ductular morphogenesis.

### Analysis of Fibrosis

The strong correlation between fibrosis and extent of ductular reaction in both experimental models and human liver disease is consistent with the concept that the terminally differentiated cholangiocyte is a crucial mediator of the fibrotic response. Following liver injury, mature cholangiocytes become “reactive cholangiocytes” and acquire the ability to secrete chemokines and cytokines that recruit inflammatory cells and contribute to the activation of portal fibroblasts and hepatic stellate cells (reviewed in Lazaridis K N, et al., (2004) Gastroenterology 127:1565-1577). The pattern of fibrosis in livers from patients with Alagille syndrome reported by Strazzabosco et al. was unusual in that it was characterized by thin septa and pericellular distribution (Fabris, L., et al., (2007) supra) in contrast to the livers from patients with Biliary Atresia in which the more stereotypical fibrotic pattern characterized by thickened fibrotic septa was detected. In addition, fibrosis correlated directly with the area of reactive ductular cells and inversely with intermediate hepatobiliary cells in Alagille syndrome livers examined by this group. These observations indicate that the extent of ductular reaction by mature reactive cholangiocytes is a determining factor in the development of fibrogenesis and that in the absence of Notch, intermediate hepatocytic or bipotential hepatobiliary cells partially compensate for the lack of mature ductular cells. Therefore, one may predict that there will be less extensive fibrosis in DDC and BDL injured livers in which Notch signaling has been inhibited in progenitor cells. The Foxl1CreERT2;Rbpj^{loxP/loxP};RosaYFP livers after BDL and DDC injury will be examined for both extent as well as cellular distribution of fibrosis and these findings will be correlated with characterization of the ductular cells with the lineage markers described above.

### Analysis of Notch Regulated Genes in Foxl1⁺Marked Progenitor Cells

At several time points following BDL or DDC induced injury, YFP⁺ cells will be isolated as described herein and used for qRT-PCR to quantify differences in cholangiocyte and hepatocytic marker profiles to further characterize differences in lineage allocation related to modulation of Notch signaling. This information will complement the in vivo immunostaining analysis and may be more sensitive for detection of differences in lineage marker expression that are not readily detectable by immunostaining.

Results

Foxl1 Promotes Cholangiocyte Proliferation after Bile Duct Induced Liver Injury Through Activation of the Canonical Wnt/β-Catenin Pathway

Mice deficient for Foxl1 exhibit increased necrosis in the bile duct ligation model (FIG. 26) and decreased cholangiocyte area (FIG. 27). Expression of the β-catenin target cyclin D1 (FIG. 28) and cholangiocyte proliferation (FIG. 29) were reduced in Foxl1 null livers post-BDL and were associated with decreased activation of wnt3a and wnt7b ligands (FIG. 30), known activators of the canonical β-catenin pathway. These findings indicate that Foxl1 activity in hepatic progenitor cells is required for cholangiocyte proliferation and liver repair through activation of the canonical wnt/β-catenin signaling pathway.

### Hepatocyte Proliferation is Delayed in Foxl1^{-/-} Livers

In the bile duct ligation model, hepatocyte proliferation is a compensatory reparative response to bile acid induced hepatocyte injury. In control livers, hepatocyte proliferation was maximal 5 days post-BDL, similar to what has been reported previously (Georgiev P, et al., (2008) Br J Surg 95:646-656). Unexpectedly, although hepatic necrosis was similar in Foxl1^{-/-} and control livers 5 days post-BDL, hepatocyte proliferation was significantly reduced in Foxl1^{-/-} livers at this time point (FIG. 31). Monga et al. recently reported that hepatocyte proliferation is also β-catenin dependent following partial hepatectomy (Tan X, et al., (2006) Gastroenterology 131:1561-1572). Without being bound by theory, this finding supports the idea that Foxl1 activation of Wnt3a and/or Wnt7b expression is essential for both progenitor cell/cholangiocyte and hepatocyte proliferation via autocrine and paracrine signaling pathways respectively. The more extensive parenchymal necrosis observed in Foxl1^{-/-} livers is likely to reflect a combination of increased bile acid induced toxicity resulting from insufficient biliary tree expansion and inadequate parenchymal repair related to decreased Wnt/β-catenin stimulation of hepatocyte proliferation.

### Notch Target Genes are Activated in BDL Injured Livers and are Enriched in the Portal Region

One can inhibit Notch signaling in Foxl1⁺ progenitor cells using conditional ablation of the Rbpj^{loxP/loxP} gene with the tamoxifen inducible Foxl1-CreERT2 mouse strain and assess for differences in liver injury and repair as well as alterations in lineage allocation in response to BDL. Although previous work had indicated a role for the Notch pathway in biliary repair in the adult liver, it was confirmed that Notch signaling targets are in fact activated in the BDL model. To determine whether the expression of members of the Notch signaling pathway was induced in nonparenchymal cells, laser capture microdissection was used to isolate portal tracts and adjacent parenchyma for gene expression analysis. As shown in FIG. 32, the Notch ligand Jagged 1 and transcriptional target Hey 1 were induced in response to BDL in the portal region. These findings, in combination with previous evidence, indicate that Notch signaling may be important for progenitor cell differentiation during postnatal biliary repair and provides additional rationale for studies in which one can investigate the function of Notch activation for biliary differentiation and ductular morphogenesis.

In summary, there is a strong rationale for the importance of the hedgehog, wnt/β-catenin and Notch signaling pathways in the regulation of hepatic progenitor function during liver repair and for Foxl1 as an essential mediator of progenitor cell expansion involving a wnt/β-catenin autocrine pathway. The feasibility of deleting individual factors involved in each of these signaling pathways with the newly developed tamoxifen inducible Foxl1-CreERT2 mouse strain and the availability of the proposed loxP mouse strains has been demonstrated. Furthermore, it has been shown that one is able to isolate Foxl1⁺YFP⁺ progenitors by FACS sorting for in vitro studies of self-renewal and differentiation. FIG. 33 provides a schematic diagram indicating how Hedgehog, β-catenin and Notch signaling pathways impact proliferation and differentiation of Foxl1⁺ reactive ductular cells and bipotential progenitors. The role of Foxl1 itself as an effector of these signaling pathways during the repair process following liver injury is also shown.

The data indicate that Sonic Hedgehog (Shh) is produced by portal fibroblasts and hepatic stellate cells as well as Foxl1⁺ progenitor cells in response to injury, and activates progenitor cell proliferation by binding to its receptor smoothened (smo). Subsequently, the Foxl1-CreERT2 mouse line will be used to inducibly delete smoothened (smo^{loxP/loxP}) in bipotential progenitor cells in injury models associated with Foxl1⁺ progenitor cell activation.

### Role of Hedgehog Signaling for Hepatic Progenitor Cell Population and Fibrogenesis in Response to Liver Injury

A study was performed using the non-inducible Foxl1Cre bred to the smo^{loxP/loxP} mice. Since Foxl1Cre is also expressed in the gut mesenchyme during development, the fact that Foxl1Cre;smo^{loxP/loxP} mice exhibited normal gut histology and growth parameters was established. Bile duct ligations were then performed on Foxl1Cre;smo^{loxP/loxP} and smo^{loxP/loxP} controls. Animals were sacrificed two weeks after surgery. Foxl1Cre;smo^{loxP/loxP} mice exhibited more extensive necrotic injury two weeks post-BDL compared to smo^{loxP/loxP} controls (FIGS. 34A and B). More extensive necrotic injury in the Foxl1Cre;smo^{loxP/loxP} livers was associated with a corresponding increase in repair as indicated by the trichrome stain for collagen (FIGS. 34C, D). These data indicate that hedgehog signals in Foxl1 expressing progenitors protect the liver against bile acid mediated acute injury but are dispensable for paracrine activation of hepatic stellate cells and portal fibroblasts.

Analysis of Injury in Response to Hedgehog Gene Inactivation in Hepatic Progenitor Cells after BDL or DDC Injury

One hour prior to sacrifice, mice will receive an intraperitoneal injection of BrdU at 50 mg/kg. At the time of liver harvest, serum will be obtained and analyzed for AST and ALT for evidence of necrotic hepatocyte injury. Total Bilirubin, alkaline phosphatase and serum bile acids will be quantified to detect differences in cholestatic injury. Livers will be fixed in paraformaldehyde and either paraffin embedded or processed in sucrose for cryopreservation. Sections will be examined for differences in necrosis, inflammation (H and E) and fibrotic injury (trichrome, Sirius red). Portions of each liver will be frozen for subsequent RNA and protein expression analyses as indicated by phenotypic changes observed on liver tissue sections. Loss of Gli 1 and Gli2 immunostaining will be used to confirm the inhibition of hedgehog signaling which could occur in the Foxl1-CreERT2smo^{loxP/loxP};RosaYFP BDL livers (Omenetti A, et al., (2008) supra). YFP staining will be used to assess the efficiency of Cre expression as an indirect measurement of loxP gene deletion. Cholangiocyte area will be quantified using IP lab image morphometry software of CK19 stained cholangiocytes and cholangiocyte proliferation will be assessed by dual labeled CK19/BrdU immunohistochemical quantification. TUNEL assay will be performed to assess for differences in apoptosis.

Constitutive Activation of Hedgehog Signaling in Foxl1Cre+ Expressing Cells after BDL or DDC Injury

Ptc^{+/-} mice display increased cholangiocyte proliferation and fibrosis after BDL (Omenetti A, et al., (2007), supra). One might hypothesize that increased progenitor cell proliferation and viability resulting from constitutive activation of hedgehog signaling in Foxl1⁺ cells will manifest itself as expanded cholangiocyte area relative to Cre negative controls. Thus, the percent YFP⁺CK19⁺, YFP⁺CK19⁻HNF-4α⁻ and YFP⁺/HNF-4α⁺ cells in ptc deleted mice after both BDL and DDC-diet induced hepatic injury will be quantified in comparison to Foxl1CreERT2;RosaYFP controls. Constitutive activation of hedgehog signaling in Foxl1⁺ reactive cholangiocytes will likely result in an increase in YFP⁺CK19⁺ cells and subsequent expansion of the descendants of these cells; YFP⁺CK19⁻HNF-4α⁻ intermediate progenitor cells and YFP⁺HNF-4α⁺ hepatocytes. In vitro data indicates that portal fibroblast and hepatic stellate cell viability can be mediated by both paracrine and autocrine signaling which leads to possibility that fibrosis will also be increased in these mice. It has been proposed that progenitor cells undergo epithelial to mesenchymal transition (EMT); however, this concept has not been tested rigorously in vivo. Because Foxl1 identifies progenitor cells and their descendents, one can investigate whether constitutive activation of the hedgehog pathway leads to progenitor cell EMT by costaining for YFP and the mesenchymal markers elastin and desmin, specific markers for portal fibroblasts and stellate cells, respectively. In the event that cholangiocyte area and proliferation are increased but there is no increase in fibrosis in Foxl1CreERT2;ptc^{loxP/loxP} mice, this will indicate that activation and survival of portal fibroblasts and/or hepatic stellate cells is not dependent upon paracrine signaling from the hepatic progenitor cell population.

### Analysis of Hedgehog Regulated Genes in Foxl1 Marked Progenitor Cells

Foxl1Cre;RosaYFP livers are subjected to BDL or DDC diet induced liver injury, Foxl1-marked cholangiocytes and hepatic progenitor cells are strongly labeled. Furthermore, YFP⁺ cells can be isolated from Foxl1Cre;RosaYFP livers after injury. YFP⁺ cells will be isolated 1, 3, and 7 days after BDL or DDC-diet induced injury from livers of mice in which hedgehog signaling in Foxl1⁺ hepatic progenitors has been inhibited or constitutively activated and quantify differences in expression of hedgehog targets in these hepatic progenitor cells. Initially focus should be on early time points post-injury in order to detect the earliest changes in hedgehog target gene expression. Analyses can be extended to later time points if warranted on the basis of the initial analyses.

Non-parenchymal cells will be isolated from the liver as described previously herein. Hepatocytes will be excluded from non-parenchymal cell (NPC) preparations by repeated low-speed (50 g) centrifugations. FACS sorting will be performed. Cells will be sorted using fluorescence-activated YFP with α-CD45 countersort to exclude hematopoietic cells.

Total RNA and protein will be isolated and reverse transcriptase quantitative PCR and immunoblot analysis will be used to identify differentially expressed hedgehog signaling targets that are the result of either hedgehog signal inhibition or activation that occur specifically in this population of cells. One could expect that decreased hedgehog activity and decreased progenitor cell proliferation in vivo will correspond to a decrease or complete inhibition in expression of known hedgehog signaling targets including Gli1, Gli2, Ptc1 and Bmi-1, a hedgehog regulated protein that plays an important role in stem cell renewal in the breast (Liu S, et al., (2006) Cancer Res 66:6063-6071). One could also expect decreased expression of Foxl1 and increased expression of Numb, a protein increased in the absence of hedgehog signaling and associated with stem cell depletion (Zhao C, et al., (2009) Nature 458:776-779). Accordingly, one could predict that levels of these signaling molecules will be increased in mice in which hepatic progenitor cell hedgehog signaling is constitutively activated (Foxl1CreERT2;ptc^{loxP/loxP};RosaYFP), with the exception of Numb which will likely be decreased in ptc deleted progenitor cells.

The results of these studies will provide important information regarding the contribution of hedgehog signaling in progenitor cells for repair in two different models associated with progenitor cell proliferation. The identification of hedgehog targets in progenitor cells isolated after in vivo injury will provide novel information regarding the effects on gene expression resulting from inhibition of hedgehog signals in these progenitor cells.

Regarding the contribution of wnt/β-catenin signaling in hepatic progenitors, tissue repair and effectors of wnt/β-catenin itself, it is known that the wnt/β-catenin signaling pathway regulates hepatoblast expansion, biliary differentiation and remodeling during liver development (Thompson M D, et al., (2007) Hepatology 45:1298-1305; Decaens T, et al., (2008) supra). However, its role in reactive cholangiocyte and progenitor cell proliferation remains unclear. Foxl1 has been identified as an important regulator of β-catenin mediated cholangiocyte proliferation in the bile duct ligation model of liver injury. The reduced expression of canonical Wnt genes in Foxl1^{-/-} mice indicates that Foxl1 regulates β-catenin by an autocrine mechanism; however, the precise mechanism by which Foxl1 controls this pathway is unknown. The tamoxifen inducible Foxl1-CreERT2 strain will be used to inducibly inactivate β-catenin (cttnb1loxP/loxP) in Foxl1⁺ reactive cholangiocytes and hepatic progenitors in the BDL and DDC models of liver injury. The consequences of inhibition of β-catenin signaling in these cell populations on tissue injury and repair will be assessed by analyzing differences in ductular proliferation, necrosis and fibrosis relative to controls. The functional importance of Foxl1 itself for activation of β-catenin mediated progenitor cell expansion will also be assessed and may contribute to the reduced cholangiocyte area in Foxl1 null mice after BDL. Foxl1 null mice will be crossed with the Foxl1-CreERT;RosaYFP reporter strain and YFP⁺ progenitor cells will be isolated following BDL or DDC induced liver injury. Clonogenic assays will be used to assess differences in the proliferative potential of Foxl1-deficient progenitors in vitro.

Foxl1^{-/-} cells will be analyzed to see whether they have restricted self-renewal by assessing clone size and number, indices of proliferation, and analysis of (β-catenin target genes including cyclin D1 and EpCAM.

Inhibition of/β-Catenin Signaling in Foxl1-Cre⁺ Expressing Cells after BDL or DDC Injury

Although nuclear localization of β-catenin is the direct indicator of (β-catenin pathway activation (reviewed in Nusse, 2008) it does not appear to be a reliable indicator in fixed liver tissues. Instead, dual immunostaining for YFP and β-catenin target genes including cyclin D1, c-myc and EpCAM will be used to assess for efficiency of β-catenin pathway inhibition in Foxl1⁺ (YFP⁺) cells. Cholangiocyte area will be quantified using IP lab image morphometry software of CK19 stained cholangiocytes and cholangiocyte proliferation will be assessed by quantification of BrdU⁺/total CK19⁺ cells. The percentage of BrdU⁺/YFP⁺ cells will also be determined to assess proliferation in Foxl1Cre⁺ cells. BrdU quantification of hepatocytes will be used to assess hepatocyte regenerative response to injury. TUNEL assay will be performed to detect differences in cholangiocyte and hepatocyte apoptosis.

Analysis of/β-Catenin Regulated Genes in Foxl1⁺Marked Progenitor Cells

YFP⁺ marked progenitor cells will be isolated 1, 3, and 7 days after BDL and DDC-diet induced injury from livers of mice in which β-catenin signaling is deleted in Foxl1⁺ hepatic progenitors (Foxl1CreERT2;cttnb1loxP/loxP;RosaYFP) and compare them to Foxl1CreERT2;RosaYFP controls. Initially focus will be on early time points post-injury in order to detect the earliest changes in β-catenin target gene expression. The time points will be extended as necessary based on the results of the initial analyses. Total RNA will be isolated from the progenitor cells and reverse transcriptase quantitative PCR will be used to quantify differences in β-catenin regulated genes that one could predict will exhibit reduced expression in the Foxl1CreERT2;cttnb1^{loxP/loxP};RosaYFP livers. These candidates include β-catenin targets previously identified in the developing liver including cyclin D1, c-myc and EpCAM. In addition, an investigation of other predicted β-catenin targets including snail (Stemmer V, et al., (2008) Oncogene 27:5075-5080), twist (Stemmer V, et al., (2008), supra) and vimentin (Yang L, et al., (2006) Cell 127:139-155) that are associated with EMT and sall4 (Bohm J, et al., (2006) Biochem Biophys Res Commun 348:898-907; Oikawa T, et al., (2009) Gastroenterology 136:1000-1011), a factor important for biliary differentiation will be undertaken. The ability to isolate an enriched population of progenitor cells from livers after BDL or DDC induced hepatic injury will increase the sensitivity of detection for these β-catenin targets. Expression of wnts themselves in progenitor cells will be quantified to investigate the potential for autocrine regulation of wnt expression by β-catenin signals in hepatic progenitor cells.

To assess the functional importance of Foxl1 for activation of β-catenin mediated progenitor cell expansion Foxl1^{-/-};RosaYFP;Foxl1CreERT2 and Foxl1^{+/+};RosaYFP;Foxl1CreERT2 controls will be obtained using the following mating scheme (FIG. 36). Animals will undergo BDL or DDC induced liver injury protocol as outlined in Table III.

TABLE III

| | Tamox | |
|---|---|---|
| Genotype | tx | BDL or DDC |
| (1) Foxl1^{+/+;} | + | 1, 3, 5, 7 |
| Foxl1CreERT2;^{;} RosaYFP | | days |
| (2)Foxl1^{-/-} | + | 1, 3, 5, 7 |
| Foxl1CreERT2;^{;} RosaYFP | | days |

To assess differences in proliferative potential of Foxl1-deficient progenitors in vitro YFP⁺ progenitor cells will be isolated initially from treatment group (1) to establish the time point for each injury model at which one can obtain the highest percentage rather than absolute numbers of colony forming progenitor cells.

Considering an in vitro progenitor assay, FACS sorting will be performed. As previously shown, after two weeks of DDC diet, approximately 0.1-0.2% of the non-parenchymal cells in the liver of Foxl1-Cre; RosaYFP mice were YFP positive. Sorted cells will be seeded at a density of 10³-10⁴ per cm² on rat Collagen I-coated Primaria (BD Falcon) tissue culture plasticware with media changes every four days. Serum free-media with defined concentrations of free fatty acids will be prepared as described (MacDonald J M, et al., (2001) Ann N Y Acad Sci 944:334-343.), and supplemented with 50 ng/ml of murine epidermal growth factor and 50 ng/ml of murine hepatocyte growth factor (Chemicon, Temecula, Calif.). Colonies, defined as organized circular clusters of at least 40 cells, will be scored on day 12.

### Identifying the Peak of Progenitor Cell Recovery

Both hepatic progenitor cells and their descendants will be labeled by YFP since a genetic mark that is produced in cells that expressed Foxl1-Cre at some point during their ontogeny is permanent. In fact, it is this feature of the system that enabled scientists to establish that Foxl1-Cre marks bipotential progenitor cells, as the descendants tracked to both hepatocytes and cholangiocytes (Sackett, 2009).

Because each model of ductular reaction has its unique properties, the response to BDL and DDC will be evaluated to identify differences in the proliferative potential of wild type and Foxl1^{-/-} progenitors. This system will be utilized to determine the length of time after BDL or DDC treatment that yields the highest percentage of progenitor cells among the YFP population. One could expect this to happen just a few days following BDL or DDC diet treatment. YFP-positive cells will be isolated and the clonogenic assay on Foxl1-CreERT2; RosaYFP (Foxl1^{+/+}) mice will be performed after 1, 3, 5, and 7 days of DDC treatment or after BDL. The time point with the highest percentage (not absolute number) of bipotential stem cells will be used for the clonogenic assay. The expression of CK19 and HNF-4-α in clones from BDL and DDC injured Foxl1CreERT2;RosaYFP livers will be characterized. One could predict that the subset of YFP⁺ cells able to form clones will be Foxl1⁺/CK19⁻/HNF-4α⁻, and are likely to be the same cells as the lacZ⁺/CK19⁻/HNF-4α⁻ detected in the lineage tracing study). Foxl1^{-/-} cells (Foxl1^{-/-}; Foxl1CreERT2;RosaYFP) will be tested to see whether they have restricted self-renewal by assessing clone size and number, indices of proliferation, and analysis of β-catenin target genes including cyclin D1 and Epcam and immunolocalization of nuclear β-catenin.

### The Role of the Notch Signaling Pathway in Hepatic Progenitor Cells for the Cell Fate Choice in New Cells Produced in Response to Injury

The identification of the Notch ligand Jagged1 as the principal gene mutated in Alagille syndrome has established an essential role for the Notch signaling pathway in biliary lineage allocation and biliary morphogenesis. There is evidence that cholangiocyte differentiation might be reversible; however, the absence of lineage tracing evidence has limited testing of this notion previously. In preliminary data it has been shown that the Notch activated transcription factors Jagged1 and Hey2 are activated periportally following BDL induced liver injury (FIG. 32). During liver development, Jagged1 ligand expression in the portal mesenchyme activates the Notch signaling pathway in bipotential hepatic progenitors required for biliary differentiation and ductular morphogenesis (Lemaigre FP. (2009) Gastroenterology). In lineage tracing experiments, it has been shown that Foxl1⁺ cells are located in close proximity to portal fibroblasts in both BDL and DDC liver injury models, leading to the idea that reactivation of Notch signaling in Foxl1⁺ progenitors may occur by a similar progenitor-mesenchymal interaction between Notch2 and Jagged1. In the recent lineage tracing study the emergence of Fox1⁺ cells negative for both hepatocyte and cholangiocyte lineage markers was detected several days after BDL or DDC induced liver injury. This Foxl1⁺CK19⁻HNF-4α⁻ population represents an intermediate cell that precedes the appearance of cells positive for the hepatocyte lineage marker HNF-4α. The small number of Foxl1⁺ intermediate cells and Foxl1⁺HNF-4α⁻ hepatocytes in comparison to Foxl1⁺CK19⁺ cells indicates that the majority of Foxl1⁺ progenitors differentiate to cholangiocytes in response to either BDL or DDC-diet induced liver injury. Taken together, these findings indicate that activation of the Notch signaling pathway in hepatic progenitor cells following BDL and DDC-diet induced liver injury is important for directing Foxl1⁺ progenitor cells to differentiate into mature cholangiocytes.

Inhibition of Notch signaling in Foxl1⁺ progenitors may result in a shift in Foxl1⁺ progenitor differentiation from the cholangiocyte to the hepatocyte lineage. The reduction in cholangiocytes might impair bile duct expansion and increased bile acid induced hepatic necrosis particularly in the BDL model which is highly dependent upon ductular cell proliferation for repair. This can be tested using conditional Notch loss-of-function studies in hepatic progenitors after BDL and DDC-diet induced hepatic injury. The consequences of Notch signal inhibition will be assessed in Foxl1⁺ progenitors on lineage allocation by analyzing differences in Foxl1⁺ cells that co-express markers of the cholangiocyte or hepatocyte lineage. The consequences of this shift in progenitor cell differentiation on the severity of acute injury and fibrosis will be investigated by examining the livers of these mice for differences in cholangiocyte area, necrosis and fibrosis. YFP⁺ hepatic progenitors will be isolated at several times points following BDL or DDC induced injury to quantify differences in cholangiocyte and hepatocytic marker profiles to further characterize differences in lineage allocation related to inhibition of Notch signaling in these cells.

Without being bound by theory, it is likely that: (1) increased progenitor cell proliferation and viability resulting from constitutive activation of hedgehog signaling in Foxl1+ cells will manifest itself as expanded cholangiocyte area relative to controls, and constitutive activation of hedgehog signaling in Foxl1⁺ reactive cholangiocytes will result in an increase in YFP⁺CK19⁺ cells and subsequent expansion of the descendants of these cells; YFP⁺CK19⁻HNF-4α⁻ intermediate progenitor cells and YFP⁺HNF-4α⁺ hepatocytes; (3) Foxl1 null progenitor cells will yield fewer and smaller sized clones and exhibit reduced expression of proliferation markers including Ki-67 and PCNA as well as β-catenin targets cyclin D1, c-myc and EpCAM, relative to wild type expressing cells; (4) inhibition of Notch signaling in Foxl1⁺ hepatic progenitor cells will prevent maturation of YFP⁺CK19⁺ progenitors to mature cholangiocyte/reactive ductular cells; and (5) inhibition of Notch signals in hepatic progenitors will result in an impaired ductular repair response due to the failure of progenitors to differentiate to reactive ductular cells.

While certain of the preferred embodiments of the present invention have been described and specifically exemplified above, it is not intended that the invention be limited to such embodiments. Various modifications may be made thereto without departing from the scope of the present invention, as set forth in the following claims.

What is claimed is:

1. An isolated adult hepatic progenitor cell which expresses forkhead winged helix factor, Foxl1 (Foxl1).

2. An isolated population of cells of claim 1, wherein said cells also express at least one marker selected from the group consisting of stem cell factor, CK19, CK7, DIWPref-I, ABCG2, c-kit, CD34, OV6, NCAM, EpCAM, Trop2, and alpha fetoprotein.

3. A composition comprising the isolated adult hepatic progenitor cells of claim 2, and at least one pharmaceutically acceptable carrier.

4. A method for preparing a device for the replacement or repair of a liver, or portion of liver, in a human comprising:
a) providing a biocompatible polymeric matrix structure in the shape of a liver or portion of a liver;
b) depositing at least one cell from the population of isolated cells of claim 2 on or in said matrix; and
c) culturing said at least one cell of step b) under suitable conditions for proliferation,
wherein said at least one cell proliferates such that said at least one cell attaches to said matrix and produces liver tissue, thereby preparing said device for the replacement or repair of a liver.

5. The method of claim 4 wherein the biocompatible polymeric matrix is formed from a material selected from the group of materials consisting of cellulose ether, cellulose, cellulosic ester, fluorinated polyethylene, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, copolymers thereof, and physical blends thereof.

6. The method of claim 4, wherein said at least one isolated adult hepatic progenitor cell is an autologous cell.

7. The method of 4, wherein said at least one isolated adult hepatic progenitor cell is an allogeneic cell.

8. The method of 4, wherein said biocompatible polymeric matrix is biodegradable.

9. A method of identifying adult hepatic progenitor cells, said method comprising:
a) obtaining a sample of hepatic cells from a subject,
b) contacting said cells with a biomolecule that specifically recognizes SEQ ID NOs: 1 or 3 or proteins encoded thereby, said biomolecule comprising a detectabe label, said biomolecule and said SEQ ID NOS 1 or 3 or sequences encoded thereby forming a detectable hybridization or immunocomplex,
c) detecting said hybridization or said immunocomplex thereby identifying adult hepatic progenitor cells, said method optionally comprising isolation of said hepatic progenitor cells.

10. The method of claim 9, comprising the step of isolating said adult hepatic progenitor cells.

11. The method of claim 10, wherein said biomolecule is an antibody and isolation is by fluorescence-activated cell sorting.

12. The method of claim 9, wherein said cells are subjected to culture conditions that cause said progenitor cells to differentiate into a hepatocyte or a cholangiocyte.

13. A method of treating hepatic injury in a patient in need thereof, said method comprising administering an effective number of isolated adult hepatic progenitor cells to said patient, wherein said adult hepatic cells express Foxl1.

14. The method of claim 13, wherein said hepatic injury is chronic hepatic injury.

15. The method of claim 13, wherein said adult hepatic cells are autologous.

16. The method of claim 13, wherein said adult hepatic progenitor cells are obtained from a second immunocompatible individual.

17. The method of claim 13, wherein said cells are optionally differentiated prior to administration.

18. A method for identifying an agent that modulates Foxl1 activity in a cell, the method comprising:
a) providing hepatic progenitor cells expressing Foxl1;
b) incubating said cells in the presence and absence of said agent;
c) determining whether said agent alters Foxl1 activity in said treated cells when compared to a control cell not exposed to the test agent, wherein a higher or lower Foxl1 activity than that of control indicates that the agent modulates Foxl1 activity.

19. The method of claim 18, wherein Foxl1 activity is selected from the group consisting of modulation of biliary cell proliferation, modulation of hepatocyte proliferation, modulation of hedgehog signaling, modulation of wnt signaling, modulation of wnt protein expression levels, modulation of notch signaling, modulation of liver cell necrosis and/or apoptosis, modulation of cyclin D activation and cholangiocyte proliferation.

20. The method of claim 19, wherein said cell is present in a mouse and said method is performed in vivo.

21. The method of claim 20, wherein expression of at least one gene has been knocked out in said mice.

22. The method of claim 20, wherein said knock out gene is selected from the group consisting of hedgehog receptor smoothened, wnt, or notch receptor.

23. A method for assessing the contribution of hepatic progenitor cells to liver homeostasis in a mouse, comprising,
a) introducing a Foxl1-diphtheria toxin receptor BAC transgene into a transgenic founder line,
b) introducing diphtheria toxin into said mice, thereby selectively ablating Foxl1 expressing cells;
c) subjecting control mice and the mice of b) to conditions that induce liver damage; and d) assessing said mice for liver repair in the presence and absence of Foxl1 expression thereby determining the contribution of Foxl1 to the proliferative response following liver injury.

24. The method of claim 23, further comprising administration of a test agent to said treated mice and assessing whether said agent augments or inhibits repair in the mice of b) relative to treated control mice.

25. A method for determining whether Foxl1 expressing hepatic progenitor cells give rise to liver cancer following genotoxic or chemotoxic damage in a mouse, comprising;
a) labeling Foxl1 expressing cells in vivo;
b) exposing said mice to a carcinogen thereby inducing tumor formation; and
c) determining whether cells in said tumor contain the Foxl1 labeled cells of
a), thereby identifying Foxl1 progenitor cells as liver cancer stem cells.


SEQUENCE LISTING (10 sequences)

SEQ ID NO: 1 - DNA, 1011 bp, Mus musculus
atgagccacctcttcagtcccccgctggccgctctggcagcctcgccgctgttgtacgtgtacagccccgagaggcctgggctgcccctggccttcgcccctgcggctgcactggccggcccgggccgcgtcgagcccccgcagaagccgccctacagctacatcgctcttatagccatggcgatccaggatgctcccgagcagagggtcacactgaacggcatctaccagttcatcatggatcgcttcccgttctaccacgacaaccgccaaggctggcagaacagcatccgccacaacctgtcgctcaacgagtgcttcgtgaaggtgccacgtgagaaggggcggccgggcaagggcagctactggaccctggacccccgctgcttggacatgtttgagaacggcaattatcggcgcaggaagaggaagcccaagcccgccgcgggttccccggaggccaagcgcacccgggtcgagccgccggagtccgaggtgggctgcgatgtagggtccccggacctggccactgcactcccgacgcgcgcgccggatcgctcccaatccccggcagtaggcaccgcgcggcccgcgctccttccctggcccggccctgagccccgggaccccgacgcggacctcacagtccagggcgcaggggcagtggcaagcggccagctccagcgtccggcacaccacctggggtcccctctgtgccccgcccccagcggctccccaaaaggttccaagtcaaagagtttcagcatcgacagcatcttggccgtgagaccgacgccagcctcaggggccgaagcaccggggatccctaagcccgtgcctggcgcgctgggctcctctctgctggctgcctcctccggcctagcgcctcccttcaacgcttcgctggtcttcgacgcgcacgtccagggcggcttttcccagctggggattcccttcctttcttattttcctctgcaggttcccgaggccacagtgcttcgtttccactaa

SEQ ID NO: 2 - protein, 336 aa, Mus musculus
MSHLFSPPLAALAASPLLYVYSPERPGLPLAFAPAAALAGPGRVEPPQKPPYSYIALIAMAIQDAPEQRVTLNGIYQFIMDRFPFYHDNRQGWQNSIRHNLSLNECFVKVPREKGRPGKGSYWTLDPRCLDMFENGNYRRRKRKPKPAAGSPEAKRTRVEPPESEVGCDVGSPDLATALPTRAPDRSQSPAVGTARPALLPWPGPEPRDPDADLTVQGAGAVASGQLQRPAHHLGSPLCPAPSGSPKGSKSKSFSIDSILAVRPTPASGAEAPGIPKPVPGALGSSLLAASSGLAPPFNASLVFDAHVQGGFSQLGIPFLSYFPLQVPEATVLRFH

SEQ ID NO: 3 - DNA, 1038 bp, Homo sapiens
atgagtcacctcttcgatccccggctgcctgccctggccgcctcgcccatgctgtatctgtacggtcccgagagacccggcctccctctggccttcgcccccgcggctgctctagctgcctcgggccgggccgagaccccgcagaagcctccctacagctacatcgcgctcatcgccatggcgatccaggacgcgcccgagcagagggtcacgctcaacggcatctaccagttcatcatggaccgcttccccttctaccacgacaaccggcagggctggcagaacagcatccgccacaacctctcgctcaacgactgcttcgtcaaggtgccccgcgagaaagggcggccgggcaagggcagctactggacgctggacccccgctgcctggacatgtttgagaacggcaactaccggcgccggaagaggaagcccaagccgggccccggggccccggaggccaagaggccccgcgccgagacgcaccagcgcagcgcggaggcgcagccggaggcggggagcggggcagggggctcgggccccgcaatctcccgcctgcaggcagcgcccgcgggcccctcgcccctcctggacggcccctctccgccggcgcccctccactggccggggaccgcgtccccgaacgaggacgctggtgacgctgcccagggcgcagcggccgtggcggtcggccaggcagcgcgcacaggggacggcccggggtcccctctgcgccccgcctcccgcagctctccgaagagctccgacaagtccaagagcttcagcatagacagcatcctggcgggaaagcagggccagaagccgccttcaggggacgaactcctagggggtgccaagcctgggcccggcggccgtctgggtgcctcgctcctggccgcctcctccagcctccgtccgcctttcaacgcttccctgatgctcgacccgcatgtccagggcggcttttaccagctcgggatccccttcctctcttatttccccctgcaggttcccgacacggtactccacttccagtaa

SEQ ID NO: 4 - protein, 345 aa, Homo sapiens
MSHLFDPRLPALAASPMLYLYGPERPGLPLAFAPAAALAASGRAETPQKPPYSYIALIAMAIQDAPEQRVTLNGIYQFIMDRFPFYHDNRQGWQNSIRHNLSLNDCFVKVPREKGRPGKGSYWTLDPRCLDMFENGNYRRRKRKPKPGPGAPEAKRPRAETHQRSAEAQPEAGSGAGGSGPAISRLQAAPAGPSPLLDGPSPPAPLHWPGTASPNEDAGDAAQGAAAVAVGQAARTGDGPGSPLRPASRSSPKSSDKSKSFSIDSILAGKQGQKPPSGDELLGGAKPGPGGRLGASLLAASSSLRPPFNASLMLDPHVQGGFYQLGIPFLSYFPLQVPDTVLHFQ

SEQ ID NO: 5 - DNA, 22 bp, Artificial Sequence
  Primer
tgccgcattccacagcatagtc

SEQ ID NO: 6 - DNA, 24 bp, Artificial Sequence
  Primer
caaagtgagttccaggacagccag

SEQ ID NO: 7 - DNA, 22 bp, Artificial Sequence
  Primer
ccaggaagcccactacaacaat

SEQ ID NO: 8 - DNA, 22 bp, Artificial Sequence
  Primer
tcgagggaggggttagagtaaa

SEQ ID NO: 9 - DNA, 21 bp, Artificial Sequence
  Primer
ccccttgtacccttcaccaat

SEQ ID NO: 10 - DNA, 21 bp, Artificial Sequence
  Primer
gaagctgcggtacaattccag